(19) United States
(12) Patent Application Publication
(10) Pub. No.: US 2009/0048219 A1
(43) Pub. Date: Feb. 19, 2009

(54) ORGANIC NITRIC OXIDE DONOR SALTS OF NONSTEROIDAL ANTIINFLAMMATORY COMPOUNDS, COMPOSITIONS AND METHODS OF USE

(75) Inventor: David S. Garvey, Dover, MA (US)

Correspondence Address: WILMERHALE/DC, 1875 PENNSYLVANIA AVE., NW, WASHINGTON, DC, 20004, US

(73) Assignee: NITROMED INC., LEXINGTON, MASSACHUSETTS, MA (US)

(21) Appl. No.: 11/914,204

(22) PCT Filed: May 23, 2006
(86) PCT No.: PCT/US06/19717

§ 371 Date: Jun. 11, 2008

Related U.S. Application Data
(60) Provisional application No. 60/683,349, filed on May 23, 2005.

(51) Int. Cl. A61K 31/56; C07D 271/04; A61K 31/4245; A61K 31/5415; C07D 417/14; C07D 401/14; C07D 271/08; A61P 1/00; A61P 17/02; A61P 27/02; A61P 11/00; A61P 9/00; A61P 35/00; C07D 413/02; A61K 31/5377; A61K 31/4439; C07D 401/02; A61K 31/454; A61K 31/496

(52) U.S. Cl. 514/171; 548/125; 514/364; 514/225.8; 544/38; 546/89; 544/141; 544/142; 544/144; 544/137; 544/140; 514/232.8; 514/236.8; 514/236.5; 514/339; 514/341; 514/343; 546/271.4; 546/275.7; 546/277.4; 546/276.7; 546/201; 546/200; 514/323; 514/326; 514/254.03; 544/367; 544/138

(57) ABSTRACT

The invention describes novel organic nitric oxide enhancing salts of nonsteroidal antiinflammatory drugs (NSAIDs) and novel compositions comprising at least one organic nitric oxide enhancing salt of an NSAID, and, optionally, at least one nitric oxide enhancing compound and/or at least one therapeutic agent. The invention also provides novel compositions and kits comprising at least one organic nitric oxide enhancing salt of an NSAID, and, optionally, at least one nitric oxide enhancing compound and/or at least one therapeutic agent. The invention also provides methods for (a) treating inflammation, pain and fever; (b) treating gastrointestinal disorders; (c) facilitating wound healing; (d) treating gastrointestinal, renal and/or respiratory toxicities resulting from the use of nonsteroidal antiinflammatory compounds; (e) treating inflammatory disease states and/or disorders; (f) treating ophthalmic disorders; (h) treating peripheral vascular diseases; (i) treating diseases resulting from oxidative stress; (j) treating endothelial dysfunctions; and (k) treating diseases caused by endothelial dysfunctions. The organic nitric oxide enhancing compounds that form salts with the NSAIDs are organic nitrates, organic nitrites, nitrosothiols, thionitrites, thionitrates, NONOates, heterocyclic nitric oxide donors and/or nitroxides. The heterocyclic nitric oxide donors are furoxans, sydnonimines, oxatriazole-5-ones and/or oxatriazole-5-imines.

### RELATED APPLICATIONS

This application claims priority under 35 USC § 119 to U.S. Application No. 60/683,349 filed May 23, 2005; the disclosures of which is incorporated by reference herein in their entirety.

### FIELD OF INVENTION

The invention describes novel organic nitric oxide enhancing salts of nonsteroidal antiinflammatory drugs (NSAIDs) and novel compositions comprising at least one organic nitric oxide enhancing salt of an NSAID, and, optionally, at least one nitric oxide enhancing compound and/or at least one therapeutic agent. The invention also provides novel compositions and kits comprising at least one organic nitric oxide enhancing salt of an NSAID, and, optionally, at least one nitric oxide enhancing compound and/or at least one therapeutic agent. The invention also provides methods for (a) treating inflammation, pain and fever; (b) treating gastrointestinal disorders; (c) facilitating wound healing; (d) treating gastrointestinal, renal and/or respiratory toxicities resulting from the use of nonsteroidal antiinflammatory compounds; (e) treating inflammatory disease states and/or disorders; (f) treating ophthalmic disorders; (h) treating peripheral vascular diseases; (i) treating diseases resulting from oxidative stress; (j) treating endothelial dysfunctions; and (k) treating diseases caused by endothelial dysfunctions. The organic nitric oxide enhancing compounds that form salts with the NSAIDs are organic nitrates, organic nitrites, nitrosothiols, thionitrites, thionitrates, NONOates, heterocyclic nitric oxide donors and/or nitroxides. The heterocyclic nitric oxide donors are furoxans, sydnonimines, oxatriazole-5-ones and/or oxatriazole-5-imines.

### BACKGROUND OF THE INVENTION

Nonsteroidal anti-inflammatory compounds (NSAIDs) are widely used for the treatment of pain, inflammation, and acute and chronic inflammatory disorders, such as, for example, osteoarthritis arthritis and rheumatoid arthritis. These compounds inhibit the activity of the enzyme cyclooxygenase (COX), also known as prostaglandin G/H synthase, which is the enzyme that converts arachidonic acid into prostanoids. The NSAIDs also inhibit the production of other prostaglandins, especially prostaglandin G₂, prostaglandin H₂ and prostaglandin E₂, thereby reducing the prostaglandin-induced pain and swelling associated with the inflammation process. The chronic use of NSAIDs has been associated with adverse effects, such as gastrointestinal ulceration and renal and respiratory toxicity. The undesirable side effects are also due to the inhibition of prostaglandin in the affected organ.

Recently two isoforms of cyclooxygenase, encoded by two distinct genes (Kujubu et al, J. Biol. Chem., 266, 12866-12872 (1991)), have been identified—a constitutive form, cyclooxygenase-1 (COX-1), and an inductive form, cyclooxygenase-2 (COX-2). It is thought that the antiinflammatory effects of NSAIDs are mediated by the inhibition of COX-2, whereas the side effects seem to be caused by the inhibition of COX-1. The NSAIDs currently on the market either inhibit both isoforms of COX with little selectivity for either isoform or are COX-1 selective.

There is still a need in the art for novel NSAIDs that do not have the adverse side effects associated with prior art compounds. There is also a need for new and improved treatments of inflammatory diseases states and disorders; and ophthalmic diseases and disorders. The invention is directed to these, as well as other, important ends.

### SUMMARY OF THE INVENTION

The invention provides novel organic nitric oxide enhancing salts of NSAIDs. The NSAIDs must contain at least one carboxylic acid group (—COOH). The organic nitric oxide enhancing compounds that form salts with the NSAIDs are organic nitrates, organic nitrites, nitrosothiols, thionitrites, thionitrates, NONOates, heterocyclic nitric oxide donors and/or nitroxides. The heterocyclic nitric oxide donors are furoxans, sydnonimines, oxatriazole-5-ones and/or oxatriazole-5-imines. The invention also provides compositions comprising the novel compounds described herein in a pharmaceutically acceptable carrier.

The invention is also based on the discovery that administering at least one organic nitric oxide enhancing salt of an NSAID, and, optionally, at least one nitric oxide enhancing compound improves the properties of the NSAID. Nitric oxide enhancing compounds include, for example, S-nitrosothiols, nitrites, nitrates, N-oxo-N-nitrosamines, furoxans, sydnonimines, oxatriazole-5-ones and/or oxatriazole-5-imines, SPM 3672, SPM 4757, SPM 5185, SPM 5186 and analogues thereof, substrates of the various isozymes of nitric oxide synthase, and nitroxides. Thus, another embodiment of the invention provides compositions comprising at least one organic nitric oxide enhancing salt of an NSAID and at least one nitric oxide enhancing compound. The invention also provides for such compositions in a pharmaceutically acceptable carrier.

The invention provides compositions comprising at least one organic nitric oxide enhancing salt of an NSAID, and, optionally, at least one nitric oxide enhancing compound and/or at least one therapeutic agent, including, but not limited to, steroids, cyclooxygenase-2 (COX-2) inhibitors, nonsteroidal antiinflammatory compounds (NSAID), 5-lipoxygenase (5-LO) inhibitors, leukotriene B₄ (LTB₄) receptor antagonists, leukotriene A₄ (LTA₄) hydrolase inhibitors, 5-HT agonists, anti-hyperlipidemic compounds, H₂ antagonists, antineoplastic agents, antiplatelet agents, thrombin inhibitors, thromboxane inhibitors, carbonic anhydrase inhibitors, decongestants, diuretics, sedating or non-sedating anti-histamines, inducible nitric oxide synthase inhibitors, opioids, analgesics, Helicobacter pylori inhibitors, phosphodiesterase inhibitors, proton pump inhibitors, isoprostane inhibitors, compounds used for the treatment of glaucoma, and combinations of two or more thereof.

The invention provides methods for (a) treating inflammation, pain and fever; (b) treating gastrointestinal disorders; (c) facilitating wound healing; (d) treating gastrointestinal, renal and/or respiratory toxicities resulting from the use of nonsteroidal antiinflammatory compounds; (e) treating inflammatory disease states and/or disorders; (f) treating ophthalmic disorders; (h) treating peripheral vascular diseases; (i) treating diseases resulting from oxidative stress; (j) treating endothelial dysfunctions; and (k) treating diseases caused by endothelial dysfunctions in a patient in need thereof comprising administering to the patient an effective amount of at least one organic nitric oxide enhancing salt of an NSAID, and, optionally, at least one therapeutic agent, such as, for example, steroids, cyclooxygenase-2 (COX-2) inhibitors, nonsteroidal antiinflammatory compounds (NSAID), 5-lipoxygenase (5-LO) inhibitors, leukotriene B₄ (LTB₄) receptor antagonists, leukotriene A₄ (LTA₄) hydrolase inhibitors, 5-HT agonists, anti-hyperlipidemic compounds, H₂ antagonists, antineoplastic agents, antiplatelet agents, thrombin inhibitors, thromboxane inhibitors, carbonic anhydrase inhibitors, decongestants, diuretics, sedating or non-sedating anti-histamines, inducible nitric oxide synthase inhibitors, opioids, analgesics, Helicobacter pylori inhibitors, phosphodiesterase inhibitors, proton pump inhibitors, isoprostane inhibitors, compounds used for the treatment of glaucoma, and combinations of two or more thereof. The methods can optionally further comprise the administration of at least one nitric oxide enhancing compound. In this embodiment of the invention, the methods can involve (i) administering the organic nitric oxide enhancing salt of the NSAIDs, (ii) administering the organic nitric oxide enhancing salt of the NSAIDs, and nitric oxide enhancing compounds, (iii) administering the organic nitric oxide enhancing salt of the NSAIDs and therapeutic agents, or (iv) administering the organic nitric oxide enhancing salt of the NSAIDs, nitric oxide enhancing compounds, and therapeutic agents. The organic nitric oxide enhancing salt of the NSAIDs, nitric oxide enhancing compounds, and/or therapeutic agents can be administered separately or as components of the same composition in one or more pharmaceutically acceptable carriers.

Another embodiment of the invention provides kits comprising at least one organic nitric oxide enhancing salt of an NSAID, and, optionally, at least one nitric oxide enhancing compound. The kit can further comprise at least one therapeutic agent, such as, for example, steroids, cyclooxygenase-2 (COX-2) inhibitors, nonsteroidal antiinflammatory compounds (NSAID), 5-lipoxygenase (5-LO) inhibitors, leukotriene B₄ (LTB₄) receptor antagonists, leukotriene A₄ (LTA₄) hydrolase inhibitors, 5-HT agonists, anti-hyperlipidemic compounds, H₂ antagonists, antineoplastic agents, antiplatelet agents, thrombin inhibitors, thromboxane inhibitors, carbonic anhydrase inhibitors, decongestants, diuretics, sedating or non-sedating anti-histamines, inducible nitric oxide synthase inhibitors, opioids, analgesics, Helicobacter pylori inhibitors, phosphodiesterase inhibitors, proton pump inhibitors, isoprostane inhibitors, compounds used for the treatment of glaucoma, and combinations of two or more thereof. The organic nitric oxide enhancing salt of the NSAID, the nitric oxide enhancing compound and/or therapeutic agent, can be separate components in the kit or can be in the form of a composition in one or more pharmaceutically acceptable carriers.

These and other aspects of the invention are described in detail herein.

### DETAILED DESCRIPTION OF THE INVENTION

As used throughout the disclosure, the following terms, unless otherwise indicated, shall be understood to have the following meanings.

“Gastrointestinal disorder” refers to any disease or disorder of the upper gastrointestinal tract of a patient including, for example, inflammatory bowel disease, Crohn's disease, gastritis, irritable bowel syndrome, constipation, ulcerative colitis, peptic ulcers, stress ulcers, bleeding ulcers, gastric hyperacidity, dyspepsia, gastroparesis, Zollinger-Ellison syndrome, gastroesophageal reflux disease, bacterial infections (including, for example, a Helicobacter Pylori associated disease), short-bowel (anastomosis) syndrome, hypersecretory states associated with systemic mastocytosis or basophilic leukemia and hyperhistaminemia, and bleeding peptic ulcers that result, for example, from neurosurgery, head injury, severe body trauma or burns.

“Upper gastrointestinal tract” refers to the esophagus, the stomach, the duodenum and the jejunum.

“Ulcers” refers to lesions of the upper gastrointestinal tract lining that are characterized by loss of tissue. Such ulcers include gastric ulcers, duodenal ulcers and gastritis.

“Cardiovascular disease or disorder” refers to any cardiovascular disease or disorder known in the art, including, but not limited to, heart failure, restenosis, hypertension (e.g. pulmonary hypertension, systolic hypertension, labile hypertension, idiopathic hypertension, low-renin hypertension, salt-sensitive hypertension, low-renin, salt-sensitive hypertension, thromboembolic pulmonary hypertension; pregnancy-induced hypertension; renovascular hypertension; hypertension-dependent end-stage renal disease, hypertension associated with cardiovascular surgical procedures, hypertension with left ventricular hypertrophy, and the like), diastolic dysfunction, coronary artery disease, myocardial infarctions, cerebral infarctions, arterial stiffness, atherosclerosis, atherogenesis, cerebrovascular disease, angina, (including chronic, stable, unstable and variant (Prinzmetal) angina pectoris), aneurysm, ischemic heart disease, cerebral ischemia, myocardial ischemia, thrombosis, platelet aggregation, platelet adhesion, smooth muscle cell proliferation, vascular or non-vascular complications associated with the use of medical devices, wounds associated with the use of medical devices, vascular or non-vascular wall damage, peripheral vascular disease, neointimal hyperplasia following percutaneous transluminal coronary angiograph, vascular grafting, coronary artery bypass surgery, thromboembolic events, post-angioplasty restenosis, coronary plaque inflammation, hypercholesterolemia, embolism, stroke, shock, arrhythmia, atrial fibrillation or atrial flutter, thrombotic occlusion and reclusion cerebrovascular incidents, left ventricular dysfunction and hypertrophy, and the like.

“Heart failure” includes, but is not limited to congestive heart failure, compensated heart failure, decompensated heart failure, and the like.

“Restenosis” is a cardiovascular disease or disorder that refers to the closure of a peripheral or coronary artery following trauma to the artery caused by an injury such as, for example, angioplasty, balloon dilation, atherectomy, laser ablation treatment or stent insertion. Restenosis can also occur following a number of invasive surgical techniques, such as, for example, transplant surgery, vein grafting, coronary artery bypass surgery, endarterectomy, heart transplantation, balloon angioplasty, atherectomy, laser ablation, endovascular stenting, and the like.

“Atherosclerosis” is a form of chronic vascular injury in which some of the normal vascular smooth muscle cells in the artery wall, which ordinarily control vascular tone regulating blood flow, change their nature and develop “cancer-like” behavior. These vascular smooth muscle cells become abnormally proliferative, secreting substances such as growth factors, tissue-degradation enzymes and other proteins, which enable them to invade and spread into the inner vessel lining, blocking blood flow and making that vessel abnormally susceptible to being completely blocked by local blood clotting, resulting in the death of the tissue served by that artery. Atherosclerotic cardiovascular disease, coronary heart disease (also known as coronary artery disease or ischemic heart disease), cerebrovascular disease and peripheral vessel disease are all common manifestations of atherosclerosis and are therefore encompassed by the terms “atherosclerosis” and “atherosclerotic disease”.

“Thromboembolic events” include, but are not limited to, ischemic stroke, transient ischemic stroke, myocardial infarction, angina pectoris, thrombosis (for example, restenosis, arterial thrombosis, coronary thrombosis, heart valve thrombosis, coronary stenosis, stent thrombosis, graft thrombosis, and first and subsequent thrombotic stroke, and the like), thromboembolism (for example, pulmonary thromboembolism, cerebral thromboembolism, and the like), thrombophlebitis, thrombocytopenia, bleeding disorders, thrombotic occlusion and reocclusion and acute vascular events. Patients who are at risk of developing thromboembolic events, may include those with a familial history of, or genetically predisposed to, thromboembolic disorders, who have had ischemic stroke, transient ischemic stroke, myocardial infarction, and those with unstable angina pectoris or chronic stable angina pectoris and patients with altered prostacyclin/thromboxane A₂ homeostasis or higher than normal thromboxane A₂ levels leading to increase risk for thromboembolism, including patients with diabetes and rheumatoid arthritis.

“Ophthalmic disorders” include, but are not limited to, glaucoma, elevated intraocular pressure, ocular pain (e.g., following corneal surgery), cataracts, ophthalmic infections, dry eye disorder, ocular hypertension, ocular bleeding, retinal diseases or disorders, presbyopia, macular degeneration, choroidal neovascularization (CNV), retinopathies, such as for example, diabetic retinopathy, vitreoretinopathy, and the like, retinitis, such as for example, cytomegalovirus (CMV) retinitis, uveitis, macular edema, neuropathies and the like.

“Ophthalmic infections” include, but are not limited, to an inflammation of the conjunctiva (conjunctivitis), inflammation of the cornea (keratitis), corneal ulcers, and the like, caused by an organisms such as, for example, Staphylococci, Streptococci, Enterococci, Bacillus, Corynebacterium, Chlamydia, Neisseria, and the like, including important species of these genus such as, for example, Staphloccus aureus, Streptococcus viridans, Staphloccus epidermidis, Streptococcus pneumoniae, staphylococci, streptococci, enterococci, and the like.

“Diseases resulting from oxidative stress” refers to any disease that involves the generation of free radicals or radical compounds, such as, for example, atherogenesis, atheromatosis, arteriosclerosis, atherosclerosis, vascular hypertrophy associated with hypertension, hyperlipoproteinaemia, normal vascular degeneration through aging, parathyroidal reactive hyperplasia, renal disease (e.g., acute or chronic), neoplastic diseases, inflammatory diseases, neurological and acute bronchopulmonary disease, tumorigenesis, ischemia-reperfusion syndrome, arthritis, sepsis, cognitive dysfunction, endotoxic shock, endotoxin-induced organ failure, and the like.

“Endothelial dysfunction” refers to the impaired ability in any physiological processes carried out by the endothelium, in particular, production of nitric oxide regardless of cause. It may be evaluated by, such as, for example, invasive techniques, such as, for example, coronary artery reactivity to acetylcholine or methacholine, and the like, or by noninvasive techniques, such as, for example, blood flow measurements, brachial artery flow dilation using cuff occlusion of the aim above or below the elbow, brachial artery ultrasonography, imaging techniques, measurement of circulating biomarkers, such as, asymmetric dimethylarginine (ADMA), and the like. For the latter measurement the endothelial-dependent flow-mediated dialation will be lower in patients diagnosed with an endothelial dysfunction.

“Methods for treating endothelial dysfunction” include, but are not limited to, treatment prior to the onset/diagnosis of a disease that is caused by or could result from endothelial dysfunction, such as, for example, atherosclerosis, hypertension, diabetes, heart failure, and the like.

“Methods for treating diseases caused by endothelial dysfunction” include, but are not limited to, the treatment of any disease resulting from the dysfunction of the endothelium, such as, for example, arteriosclerosis, heart failure, hypertension, cardiovascular diseases, cerebrovascular diseases, renovascular diseases, mesenteric vascular diseases, pulmonary vascular diseases, ocular vascular diseases, peripheral vascular diseases, peripheral ischemic diseases, and the like.

“Therapeutic agent” includes any therapeutic agent that can be used to treat or prevent the diseases described herein. “Therapeutic agents” include, for example, steroids, COX-2 inhibitors, nonsteroidal antiinflammatory compounds, 5-lipoxygenase inhibitors, leukotriene B₄ receptor antagonists, leukotriene A₄ hydrolase inhibitors, 5-HT agonists, 3-hydroxy-3-methylglutaryl coenzyme A inhibitors, H₂ antagonists, antineoplastic agents, antiplatelet agents, thrombin inhibitors, thromboxane inhibitors, carbonic anhydrase inhibitors, decongestants, diuretics, sedating or non-sedating anti-histamines, inducible nitric oxide synthase inhibitors, opioids, analgesics, Helicobacter pylori inhibitors, phosphodiesterase inhibitors, proton pump inhibitors, isoprostane inhibitors, compounds used for the treatment of glaucoma, and the like. Therapeutic agent includes the pro-drugs and pharmaceutical derivatives thereof including but not limited to the corresponding nitrosated and/or nitrosylated derivatives and/or therapeutic agents containing at least one heterocyclic nitric oxide donor group. Although nitric oxide enhancing compounds have therapeutic activity, the term “therapeutic agent” does not include the nitric oxide enhancing compounds described herein, since nitric oxide enhancing compounds are separately defined.

“Prodrug” refers to a compound that is made more active in vivo.

“NSAID” refers to a nonsteroidal anti-inflammatory compound or a nonsteroidal anti-inflammatory drug. NSAIDs inhibit cyclooxygenase, the enzyme responsible for the biosyntheses of the prostaglandins and certain autocoid inhibitors, including inhibitors of the various isozymes of cyclooxygenase (including but not limited to cyclooxygenase-1 and -2), and as inhibitors of both cyclooxygenase and lipoxygenase.

“Cyclooxygenase-2 (COX-2) selective inhibitor” refers to a compound that selectively inhibits the cyclooxygenase-2 enzyme over the cyclooxygenase-1 enzyme. In one embodiment, the compound has a cyclooxygenase-2 IC₅₀ of less than about 2 μM and a cyclooxygenase-1 IC₅₀ of greater than about 5 μM, in the human whole blood COX-2 assay (as described in Brideau et al., Inflamm Res., 45: 68-74 (1996)) and also has a selectivity ratio of cyclooxygenase-2 inhibition over cyclooxygenase-1 inhibition of at least 10, and preferably of at least 40. In another embodiment, the compound has a cyclooxygenase-1 IC₅₀ of greater than about 1 μM, and preferably of greater than 20 μM. The compound can also inhibit the enzyme, lipoxygenase. Such selectivity may indicate an ability to reduce the incidence of common NSAID-induced side effects.

“Proton pump inhibitor” refers to any compound that reversibly or irreversibly blocks gastric acid secretion by inhibiting the H⁺/K⁺-ATP ase enzyme system at the secretory surface of the gastric parietal cell.

“Thromboxane inhibitor” refers to any compound that reversibly or irreversibly inhibits thromboxane synthesis, and includes compounds which are the so-called thromboxane A₂ receptor antagonists, thromboxane A₂ antagonists, thromboxane A₂/prostaglandin endoperoxide antagonists, thromboxane receptor (TP) antagonists, thromboxane antagonists, thromboxane synthase inhibitors, and dual acting thromboxane synthase inhibitors and thromboxane receptor antagonists. The characteristics of the preferred thromboxane inhibitor should include the suppression of thromboxane A₂ formation (thromboxane synthase inhibitors) and/or blockade of thromboxane A₂ and prostaglandin H₂ platelet and vessel wall (thromboxane receptor antagonists). The effects should block platelet activation and therefore platelet function.

“Thromboxane A₂ receptor antagonist” refers to any compound that reversibly or irreversibly blocks the activation of any thromboxane A₂ receptor.

“Thromboxane synthase inhibitor” refers to any compound that reversibly or irreversibly inhibits the enzyme thromboxane synthesis thereby reducing the formation of thromboxane A₂. Thromboxane synthase inhibitors may also increase the synthesis of antiaggregatory prostaglandins including prostacyclin and prostaglandin D₂. Thromboxane A₂ receptor antagonists and thromboxane synthase inhibitors and can be identified using the assays described in Tai, Methods of Enzymology, Vol. 86, 110-113 (1982); Hall, Medicinal Research Reviews, 11:503-579 (1991) and Coleman et al., Pharmacol Rev., 46: 205-229 (1994) and references therein, the disclosures of each of which are incorporated by reference herein in their entirety.

“Dual acting thromboxane receptor antagonist and thromboxane synthase inhibitor” refers to any compound that simultaneously acts as a thromboxane A₂ receptor antagonist and a thromboxane synthase inhibitor.

“Thrombin inhibitors” refers to and includes compounds that inhibit hydrolytic activity of thrombin, including the catalytic conversion of fibrinogen to fibrin, activation of Factor V to Va, Factor VIII to VIIIa, Factor XIII to XIIIa and platelet activation. Thrombin inhibitors may be identified using assays described in Lewis et at., Thrombosis Research. 70: 173-190 (1993).

“Platelet aggregation” refers to the binding of one or more platelets to each other. Platelet aggregation is commonly referred to in the context of generalized atherosclerosis, not with respect to platelet adhesion on vasculature damaged as a result of physical injury during a medical procedure. Platelet aggregation requires platelet activation which depends on the interaction between the ligand and its specific platelet surface receptor.

“Platelet activation” refers either to the change in conformation (shape) of a cell, expression of cell surface proteins (e.g., the IIb/IIIa receptor complex, loss of GPIb surface protein), and secretion of platelet derived factors (e.g., serotonin, growth factors).

“Patient” refers to animals, preferably mammals, most preferably humans, and includes males and females, and children and adults.

“Effective amount” refers to the amount of the compound and/or composition that is effective to achieve its intended purpose.

“Transdermal” refers to the delivery of a compound by passage through the skin and into the blood stream.

“Transmucosal” refers to delivery of a compound by passage of the compound through the mucosal tissue and into the blood stream.

“Penetration enhancement” or “permeation enhancement” refers to an increase in the permeability of the skin or mucosal tissue to a selected pharmacologically active compound such that the rate at which the compound permeates through the skin or mucosal tissue is increased.

“Carriers” or “vehicles” refers to carrier materials suitable for compound administration and include any such material known in the art such as, for example, any liquid, gel, solvent, liquid diluent, solubilizer, or the like, which is non-toxic and which does not interact with any components of the composition in a deleterious manner.

“Sustained release” refers to the release of an active compound and/or composition such that the blood levels of the active compound are maintained within a desirable therapeutic range over a period of time. The sustained release formulation can be prepared using any conventional method known to one skilled in the art to obtain the desired release characteristics.

“Nitric oxide enhancing” refers to compounds and functional groups which, under physiological conditions can increase endogenous nitric oxide. Nitric oxide enhancing compounds include, but are not limited to, nitric oxide releasing compounds, nitric oxide donating compounds, nitric oxide donors, radical scavenging compounds and/or reactive oxygen species scavenger compounds. In one embodiment the radical scavenging compound contains a nitroxide group.

“Nitroxide group” refers to compounds that have the ability to mimic superoxide dimutase and catalase and act as radical scavengers, or react with superoxide or other reactive oxygen species via a stable aminoxyl radical i.e. N-oxide.

“Nitric oxide adduct” or “NO adduct” refers to compounds and functional groups which, under physiological conditions, can donate, release and/or directly or indirectly transfer any of the three redox forms of nitrogen monoxide (NO⁺, NO⁻, NO.), such that the biological activity of the nitrogen monoxide species is expressed at the intended site of action.

“Nitric oxide releasing” or “nitric oxide donating” refers to methods of donating, releasing and/or directly or indirectly transferring any of the three redox forms of nitrogen monoxide (NO⁺, NO⁻, NO.), such that the biological activity of the nitrogen monoxide species is expressed at the intended site of action.

“Nitric oxide donor” or “NO donor” refers to compounds that donate, release and/or directly or indirectly transfer a nitrogen monoxide species, and/or stimulate the endogenous production of nitric oxide or endothelium-derived relaxing factor (EDRF) in vivo and/or elevate endogenous levels of nitric oxide or EDRF in vivo and/or are oxidized to produce nitric oxide and/or are substrates for nitric oxide synthase and/or cytochrome P450. “NO donor” also includes compounds that are precursors of L-arginine, inhibitors of the enzyme arginase and nitric oxide mediators.

“Heterocyclic nitric oxide donor” refers to a trisubstituted 5-membered ring comprising two or three nitrogen atoms and at least one oxygen atom. The heterocyclic nitric oxide donor is capable of donating and/or releasing a nitrogen monoxide species upon decomposition of the heterocyclic ring. Exemplary heterocyclic nitric oxide donors include oxatriazol-5-ones, oxatriazol-5-imines, sydnonimines, furoxans, and the like.

“Alkyl” refers to a lower alkyl group, a substituted lower alkyl group, a haloalkyl group, a hydroxyalkyl group, an alkenyl group, a substituted alkenyl group, an alkynyl group, a bridged cycloalkyl group, a cycloalkyl group or a heterocyclic ring, as defined herein. An alkyl group may also comprise one or more radical species, such as, for example a cycloalkylalkyl group or a heterocyclicalkyl group.

“Lower alkyl” refers to branched or straight chain acyclic alkyl group comprising one to about ten carbon atoms (preferably one to about eight carbon atoms, more preferably one to about six carbon atoms). Exemplary lower alkyl groups include methyl, ethyl, n-propyl, isopropyl, n-butyl, isobutyl, sec-butyl, t-butyl, pentyl, neopentyl, iso-amyl, hexyl, octyl, and the like.

“Substituted lower alkyl” refers to a lower alkyl group, as defined herein, wherein one or more of the hydrogen atoms have been replaced with one or more R¹⁰⁰ groups, wherein each R¹⁰⁰ is independently a hydroxy, an ester, an amidyl, an oxo, a carboxyl, a carboxamido, a halo, a cyano, a nitrate, a nitrite, a thionitrate, a thionitrite or an amino group, as defined herein.

“Haloalkyl” refers to a lower alkyl group, an alkenyl group, an alkynyl group, a bridged cycloalkyl group, a cycloalkyl group or a heterocyclic ring, as defined herein, to which is appended one or more halogens, as defined herein. Exemplary haloalkyl groups include trifluoromethyl, chloromethyl, 2-bromobutyl, 1-bromo-2-chloro-pentyl, and the like.

“Alkenyl” refers to a branched or straight chain C₂-C₁₀ hydrocarbon (preferably a C₂-C₈ hydrocarbon, more preferably a C₂-C₆ hydrocarbon) that can comprise one or more carbon-carbon double bonds. Exemplary alkenyl groups include propylenyl, buten-1-yl, isobutenyl, penten-1-yl, 2,2-methylbuten-1-yl, 3-methylbuten-1-yl, hexan-1-yl, hepten-1-yl, octen-1-yl, and the like.

“Lower alkenyl” refers to a branched or straight chain C₂-C₄ hydrocarbon that can comprise one or two carbon-carbon double bonds.

“Substituted alkenyl” refers to a branched or straight chain C₂-C₁₀ hydrocarbon (preferably a C₂-C₈ hydrocarbon, more preferably a C₂-C₆ hydrocarbon) which can comprise one or more carbon-carbon double bonds, wherein one or more of the hydrogen atoms have been replaced with one or more R¹⁰⁰ groups, wherein each R¹⁰⁰ is independently a hydroxy, an oxo, a carboxyl, a carboxamido, a halo, a cyano or an amino group, as defined herein.

“Alkynyl” refers to an unsaturated acyclic C₂-C₁₀ hydrocarbon (preferably a C₂-C₈ hydrocarbon, more preferably a C₂-C₆ hydrocarbon) that can comprise one or more carbon-carbon triple bonds. Exemplary alkynyl groups include ethynyl, propynyl, butyn-1-yl, butyn-2-yl, pentyl-1-yl, pentyl-2-yl, 3-methylbutyn-1-yl, hexyl-1-yl, hexyl-2-yl, hexyl-3-yl, 3,3-dimethyl-butyn-1-yl, and the like.

“Bridged cycloalkyl” refers to two or more cycloalkyl groups, heterocyclic groups, or a combination thereof fused via adjacent or non-adjacent atoms. Bridged cycloalkyl groups can be unsubstituted or substituted with one, two or three substituents independently selected from alkyl, alkoxy, amino, alkylamino, dialkylamino, hydroxy, halo, carboxyl, alkylcarboxylic acid, aryl, amidyl, ester, alkylcarboxylic ester, carboxamido, alkylcarboxamido, oxo and nitro. Exemplary bridged cycloalkyl groups include adamantyl, decahydronapthyl, quinuclidyl, 2,6-dioxabicyclo(3.3.0)octane, 7-oxabicyclo(2.2.1)heptyl, 8-azabicyclo(3,2,1)oct-2-enyl and the like.

“Cycloalkyl” refers to a saturated or unsaturated cyclic hydrocarbon comprising from about 3 to about 10 carbon atoms. Cycloalkyl groups can be unsubstituted or substituted with one, two or three substituents independently selected from alkyl, alkoxy, amino, alkylamino, dialkylamino, arylamino, diarylamino, alkylarylamino, aryl, amidyl, ester, hydroxy, halo, carboxyl, alkylcarboxylic acid, alkylcarboxylic ester, carboxamido, alkylcarboxamido, oxo, alkylsulfinyl, and nitro. Exemplary cycloalkyl groups include cyclopropyl, cyclobutyl, cyclopentyl, cyclohexyl, cyclohexenyl, cyclohepta-1,3-dienyl, and the like.

“Heterocyclic ring or group” refers to a saturated or unsaturated cyclic hydrocarbon group having about 2 to about 10 carbon atoms (preferably about 4 to about 6 carbon atoms) where 1 to about 4 carbon atoms are replaced by one or more nitrogen, oxygen and/or sulfur atoms. Sulfur may be in the thio, sulfinyl or sulfonyl oxidation state. The heterocyclic ring or group can be fused to an aromatic hydrocarbon group. Heterocyclic groups can be unsubstituted or substituted with one, two or three substituents independently selected from alkyl, alkoxy, amino, alkylthio, aryloxy, arylthio, arylalkyl, hydroxy, oxo, thial, halo, carboxyl, carboxylic ester, alkylcarboxylic acid, alkylcarboxylic ester, aryl, arylcarboxylic acid, arylcarboxylic ester, amidyl, ester, alkylcarbonyl, arylcarbonyl, alkylsulfinyl, carboxamido, alkylcarboxamido, arylcarboxamido, sulfonic acid, sulfonic ester, sulfonamide nitrate and nitro. Exemplary heterocyclic groups include pyrrolyl, furyl, thienyl, 3-pyrrolinyl, 4,5,6-trihydro-2H-pyranyl, pyridinyl, 1,4-dihydropyridinyl, pyrazolyl, triazolyl, pyrimidinyl, pyridazinyl, oxazolyl, thiazolyl, imidazolyl, indolyl, thiophenyl, furanyl, tetrahydrofuranyl, tetrazolyl, pyrrolinyl, pyrrolindinyl, oxazolindinyl 1,3-dioxolanyl, imidazolinyl, imidazolindinyl, pyrazolinyl, pyrazolidinyl, isoxazolyl, isothiazolyl, 1,2,3-oxadiazolyl, 1,2,3-triazolyl, 1,3,4-thiadiazolyl, 2H-pyranyl, 4H-pyranyl, piperidinyl, 1,4-dioxanyl, morpholinyl, 1,4-dithianyl, thiomorpholinyl, pyrazinyl, piperazinyl, 1,3,5-triazinyl, 1,3,5-trithianyl, benzo(b)thiophenyl, benzimidazolyl, benzothiazolinyl, quinolinyl, 2,6-dioxabicyclo(3.3.0)octane, and the like.

“Heterocyclic compounds” refer to mono- and polycyclic compounds comprising at least one aryl or heterocyclic ring.

“Aryl” refers to a monocyclic, bicyclic, carbocyclic or heterocyclic ring system comprising one or two aromatic rings. Exemplary aryl groups include phenyl, pyridyl, napthyl, quinoyl, tetrahydronaphthyl, furanyl, indanyl, indenyl, indoyl, and the like. Aryl groups (including bicyclic aryl groups) can be unsubstituted or substituted with one, two or three substituents independently selected from alkyl, alkoxy, alkylthio, amino, alkylamino, dialkylamino, arylamino, diarylamino, alkylarylamino, halo, cyano, alkylsulfinyl, hydroxy, carboxyl, carboxylic ester, alkylcarboxylic acid, alkylcarboxylic ester, aryl, arylcarboxylic acid, arylcarboxylic ester, alkylcarbonyl, arylcarbonyl, amidyl, ester, carboxamido, alkylcarboxamido, carbomyl, sulfonic acid, sulfonic ester, sulfonamido and nitro. Exemplary substituted aryl groups include tetrafluorophenyl, pentafluorophenyl, sulfonamide, alkylsulfonyl, arylsulfonyl, and the like.

“Cycloalkenyl” refers to an unsaturated cyclic C₂-C₁₀ hydrocarbon (preferably a C₂-C₈ hydrocarbon, more preferably a C₂-C₆ hydrocarbon) which can comprise one or more carbon-carbon double bonds.

“Alkylaryl” refers to an alkyl group, as defined herein, to which is appended an aryl group, as defined herein. Exemplary alkylaryl groups include benzyl, phenylethyl, hydroxybenzyl, fluorobenzyl, fluorophenylethyl, and the like.

“Arylalkyl” refers to an aryl radical, as defined herein, attached to an alkyl radical, as defined herein. Exemplary arylalkyl groups include benzyl, phenylethyl, 4-hydroxybenzyl, 3-fluorobenzyl, 2-fluorophenylethyl, and the like.

“Arylalkenyl” refers to an aryl radical, as defined herein, attached to an alkenyl radical, as defined herein. Exemplary arylalkenyl groups include styryl, propenylphenyl, and the like.

“Cycloalkylalkyl” refers to a cycloalkyl radical, as defined herein, attached to an alkyl radical, as defined herein.

“Cycloalkylalkoxy” refers to a cycloalkyl radical, as defined herein, attached to an alkoxy radical, as defined herein.

“Cycloalkylalkylthio” refers to a cycloalkyl radical, as defined herein, attached to an alkylthio radical, as defined herein.

“Heterocyclicalkyl” refers to a heterocyclic ring radical, as defined herein, attached to an alkyl radical, as defined herein.

“Arylheterocyclic ring” refers to a bi- or tricyclic ring comprised of an aryl ring, as defined herein, appended via two adjacent carbon atoms of the aryl ring to a heterocyclic ring, as defined herein. Exemplary arylheterocyclic rings include dihydroindole, 1,2,3,4-tetra-hydroquinoline, and the like.

“Alkylheterocyclic ring” refers to a heterocyclic ring radical, as defined herein, attached to an alkyl radical, as defined herein. Exemplary alkylheterocyclic rings include 2-pyridylmethyl, 1-methylpiperidin-2-one-3-methyl, and the like.

“Alkoxy” refers to R₅₀O—, wherein R₅₀ is an alkyl group, as defined herein (preferably a lower alkyl group or a haloalkyl group, as defined herein). Exemplary alkoxy groups include methoxy, ethoxy, t-butoxy, cyclopentyloxy, trifluoromethoxy, and the like.

“Aryloxy” refers to R₅₅O—, wherein R₅₅ is an aryl group, as defined herein. Exemplary arylkoxy groups include napthyloxy, quinolyloxy, isoquinolizinyloxy, and the like.

“Alkylthio” refers to R₅₀S—, wherein R₅₀ is an alkyl group, as defined herein.

“Lower alkylthio” refers to a lower alkyl group, as defined herein, appended to a thio group, as defined herein.

“Arylalkoxy” or “alkoxyaryl” refers to an alkoxy group, as defined herein, to which is appended an aryl group, as defined herein. Exemplary arylalkoxy groups include benzyloxy, phenylethoxy, chlorophenylethoxy, and the like.

“Arylalklythio” refers to an alkylthio group, as defined herein, to which is appended an aryl group, as defined herein. Exemplary arylalklythio groups include benzylthio, phenylethylthio, chlorophenylethylthio, and the like.

“Arylalklythioalkyl” refers to an arylalkylthio group, as defined herein, to which is appended an alkyl group, as defined herein. Exemplary arylalklythioalkyl groups include benzylthiomethyl, phenylethylthiomethyl, chlorophenylethylthioethyl, and the like.

“Alkylthioalkyl” refers to an alkylthio group, as defined herein, to which is appended an alkyl group, as defined herein. Exemplary alkylthioalkyl groups include alkylthiomethyl, ethylthiomethyl, trifluoroethylthiomethyl, and the like.

“Alkoxyalkyl” refers to an alkoxy group, as defined herein, appended to an alkyl group, as defined herein. Exemplary alkoxyalkyl groups include methoxymethyl, methoxyethyl, isopropoxymethyl, and the like.

“Alkoxyhaloalkyl” refers to an alkoxy group, as defined herein, appended to a haloalkyl group, as defined herein. Exemplary alkoxyhaloalkyl groups include 4-methoxy-2-chlorobutyl and the like.

“Cycloalkoxy” refers to R₅₄O—, wherein R₅₄ is a cycloalkyl group or a bridged cycloalkyl group, as defined herein. Exemplary cycloalkoxy groups include cyclopropyloxy, cyclopentyloxy, cyclohexyloxy, and the like.

“Cycloalkylthio” refers to R₅₄S—, wherein R₅₄ is a cycloalkyl group or a bridged cycloalkyl group, as defined herein. Exemplary cycloalkylthio groups include cyclopropylthio, cyclopentylthio, cyclohexylthio, and the like.

“Haloalkoxy” refers to an alkoxy group, as defined herein, in which one or more of the hydrogen atoms on the alkoxy group are substituted with halogens, as defined herein. Exemplary haloalkoxy groups include 1,1,1-trichloroethoxy, 2-bromobutoxy, and the like.

“Hydroxy” refers to —OH.

“Oxy” refers to —O—

“Oxo” refers to ═O.

“Oxylate” refers to —O⁻R₇₇⁺ wherein R₇₇ is an organic or inorganic cation.

“Thiol” refers to —SH.

“Thio” refers to —S—.

“Oxime” refers to ═N—OR₈₁ wherein R₈₁ is a hydrogen, an alkyl group, an aryl group, an alkylsulfonyl group, an arylsulfonyl group, a carboxylic ester, an alkylcarbonyl group, an arylcarbonyl group, a carboxamido group, an alkoxyalkyl group or an alkoxyaryl group.

“Hydrazone” refers to ═N—N(R₈₁)(R′₈₁) wherein R′₈₁ is independently selected from R₈₁, and R₈₁ is as defined herein.

“Hydrazino” refers to H₂N—N(H)—.

“Organic cation” refers to a positively charged organic ion. Exemplary organic cations include alkyl substituted ammonium cations, and the like.

“Inorganic cation” refers to a positively charged metal ion. Exemplary inorganic cations include Group I metal cations such as for example, sodium, potassium, magnesium, calcium, and the like.

“Hydroxyalkyl” refers to a hydroxy group, as defined herein, appended to an alkyl group, as defined herein.

“Nitrate” refers to —O—NO₂ i.e. oxidized nitrogen.

“Nitrite” refers to —O—NO i.e. oxidized nitrogen.

“Thionitrate” refers to —S—NO₂.

“Thionitrite” and “nitrosothiol” refer to —S—NO.

“Nitro” refers to the group —NO₂ and “nitrosated” refers to compounds that have been substituted therewith.

“Nitroso” refers to the group —NO and “nitrosylated” refers to compounds that have been substituted therewith.

“Nitrile” and “cyano” refer to —CN.

“Halogen” or “halo” refers to iodine (I), bromine (Br), chlorine (Cl), and/or fluorine (F).

“Imine” refers to —C(═N—R₅₁)— wherein R₅₁ is a hydrogen atom, an alkyl group, an aryl group or an arylheterocyclic ring, as defined herein

“Amine” refers to any organic compound that contains at least one basic nitrogen atom.

“Amino” refers to —NH₂, an alkylamino group, a dialkylamino group, an arylamino group, a diarylamino group, an alkylarylamino group or a heterocyclic ring, as defined herein.

“Alkylamino” refers to R₅₀NH—, wherein R₅₀ is an alkyl group, as defined herein. Exemplary alkylamino groups include methylamino, ethylamino, butylamino, cyclohexylamino, and the like.

“Arylamino” refers to R₅₅NH—, wherein R₅₅ is an aryl group, as defined herein.

“Dialkylamino” refers to R₅₂R₅₃N—, wherein R₅₂ and R₅₃ are each independently an alkyl group, as defined herein. Exemplary dialkylamino groups include dimethylamino, diethylamino, methyl propargylamino, and the like.

“Diarylamino” refers to R₅₅R₆₀N—, wherein R₅₅ and R₆₀ are each independently an aryl group, as defined herein.

“Alkylarylamino” or “arylalkylamino” refers to R₅₂R₅₅N—, wherein R₅₂ is an alkyl group, as defined herein, and R₅₅ is an aryl group, as defined herein.

“Alkylarylalkylamino” refers to R₅₂R₇₉N—, wherein R₅₂ is an alkyl group, as defined herein, and R₇₉ is an arylalkyl group, as defined herein.

“Alkylcycloalkylamino” refers to R₅₂R₈₀N—, wherein R₅₂ is an alkyl group, as defined herein, and R₈₀ is a cycloalkyl group, as defined herein.

“Aminoalkyl” refers to an amino group, an alkylamino group, a dialkylamino group, an arylamino group, a diarylamino group, an alkylarylamino group or a heterocyclic ring, as defined herein, to which is appended an alkyl group, as defined herein. Exemplary aminoalkyl groups include dimethylaminopropyl, diphenylaminocyclopentyl, methylaminomethyl, and the like.

“Aminoaryl” refers to an aryl group to which is appended an alkylamino group, an arylamino group or an arylalkylamino group. Exemplary aminoaryl groups include anilino, N-methylanilino, N-benzylanilino, and the like.

“Sulfinyl” refers to —S(O)—.

“Methanthial” refers to —C(S)—.

“Thial” refers to ═S.

“Sulfonyl” refers to —S(O)₂⁻.

“Sulfonic acid” refers to —S(O)₂OR₇₆, wherein R₇₆ is a hydrogen, an organic cation or an inorganic cation, as defined herein.

“Alkylsulfonic acid” refers to a sulfonic acid group, as defined herein, appended to an alkyl group, as defined herein.

“Arylsulfonic acid” refers to a sulfonic acid group, as defined herein, appended to an aryl group, as defined herein.

“Sulfonic ester” refers to —S(O)₂OR₅₈, wherein R₅₈ is an alkyl group, an aryl group, or an aryl heterocyclic ring, as defined herein.

“Sulfonamido” refers to —S(O)₂—N(R₅₁)(R₅₇), wherein R₅₁ and R₅₇ are each independently a hydrogen atom, an alkyl group, an aryl group or an arylheterocyclic ring, as defined herein, or R₅₁ and R₅₇ when taken together are a heterocyclic ring, a cycloalkyl group or a bridged cycloalkyl group, as defined herein.

“Alkylsulfonamido” refers to a sulfonamido group, as defined herein, appended to an alkyl group, as defined herein.

“Arylsulfonamido” refers to a sulfonamido group, as defined herein, appended to an aryl group, as defined herein.

“Alkylthio” refers to R₅₀S—, wherein R₅₀ is an alkyl group, as defined herein (preferably a lower alkyl group, as defined herein).

“Arylthio” refers to R₅₅S—, wherein R₅₅ is an aryl group, as defined herein.

“Arylalkylthio” refers to an aryl group, as defined herein, appended to an alkylthio group, as defined herein.

“Alkylsulfinyl” refers to R₅₀—S(O)—, wherein R₅₀ is an alkyl group, as defined herein.

“Alkylsulfonyl” refers to R₅₀—S(O)₂—, wherein R₅₀ is an alkyl group, as defined herein.

“Alkylsulfonyloxy” refers to R₅₀—S(O)₂—O—, wherein R₅₀ is an alkyl group, as defined herein.

“Arylsulfinyl” refers to R₅₅—S(O)—, wherein R₅₅ is an aryl group, as defined herein.

“Arylsulfonyl” refers to R₅₅—S(O)₂—, wherein R₅₅ is an aryl group, as defined herein.

“Arylsulfonyloxy” refers to R₅₅—S(O)₂—O—, wherein R₅₅ is an aryl group, as defined herein.

“Amidyl” refers to R₅₁C(O)N(R₅₇)— wherein R₅₁ and R₅₇ are each independently a hydrogen atom, an alkyl group, an aryl group or an arylheterocyclic ring, as defined herein.

“Ester” refers to R₅₁C(O)R₈₂— wherein R₅₁ is a hydrogen atom, an alkyl group, an aryl group or an arylheterocyclic ring, as defined herein and R₈₂ is oxygen or sulfur.

“Carbamoyl” refers to —O—C(O)N(R₅₁)(R₅₇), wherein R₅₁ and R₅₇ are each independently a hydrogen atom, an alkyl group, an aryl group or an arylheterocyclic ring, as defined herein, or R₅₁ and R₅₇ taken together are a heterocyclic ring, a cycloalkyl group or a bridged cycloalkyl group, as defined herein.

“Carboxyl” refers to —C(O)OR₇₆, wherein R₇₆ is a hydrogen, an organic cation or an inorganic cation, as defined herein.

“Carbonyl” refers to —C(O)—.

“Alkylcarbonyl” refers to R₅₂—C(O)—, wherein R₅₂ is an alkyl group, as defined herein.

“Arylcarbonyl” refers to R₅₅—C(O)—, wherein R₅₅ is an aryl group, as defined herein.

“Arylalkylcarbonyl” refers to R₅₅—R₅₂—C(O)—, wherein R₅₅ is an aryl group, as defined herein, and R₅₂ is an alkyl group, as defined herein.

“Alkylarylcarbonyl” refers to R₅₂—R₅₅—C(O)—, wherein R₅₅ is an aryl group, as defined herein, and R₅₂ is an alkyl group, as defined herein.

“Heterocyclicalkylcarbonyl” refer to R₇₈C(O)— wherein R₇₈ is a heterocyclicalkyl group, as defined herein.

“Carboxylic ester” refers to —C(O)OR₅₈, wherein R₅₈ is an alkyl group, an aryl group or an aryl heterocyclic ring, as defined herein.

“Alkylcarboxylic acid” and “alkylcarboxyl” refer to an alkyl group, as defined herein, appended to a carboxyl group, as defined herein.

“Alkylcarboxylic ester” refers to an alkyl group, as defined herein, appended to a carboxylic ester group, as defined herein.

“Alkyl ester” refers to an alkyl group, as defined herein, appended to an ester group, as defined herein.

“Arylcarboxylic acid” refers to an aryl group, as defined herein, appended to a carboxyl group, as defined herein.

“Arylcarboxylic ester” and “arylcarboxyl” refer to an aryl group, as defined herein, appended to a carboxylic ester group, as defined herein.

“Aryl ester” refers to an aryl group, as defined herein, appended to an ester group, as defined herein.

“Carboxamido” refers to —C(O)N(R₅₁)(R₅₇), wherein R₅₁ and R₅₇ are each independently a hydrogen atom, an alkyl group, an aryl group or an arylheterocyclic ring, as defined herein, or R₅₁ and R₅₇ when taken together are a heterocyclic ring, a cycloalkyl group or a bridged cycloalkyl group, as defined herein.

“Alkylcarboxamido” refers to an alkyl group, as defined herein, appended to a carboxamido group, as defined herein.

“Arylcarboxamido” refers to an aryl group, as defined herein, appended to a carboxamido group, as defined herein.

“Urea” refers to —N(R₅₉)—C(O)N(R₅₁)(R₅₇) wherein R₅₁, R₅₇, and R₅₉ are each independently a hydrogen atom, an alkyl group, an aryl group or an arylheterocyclic ring, as defined herein, or R₅₁ and R₅₇ taken together are a heterocyclic ring, a cycloalkyl group or a bridged cycloalkyl group, as defined herein.

“Phosphoryl” refers to —P(R₇₀)(R₇₁)(R₇₂), wherein R₇₀ is a lone pair of electrons, thial or oxo, and R₇₁ and R₇₂ are each independently a covalent bond, a hydrogen, a lower alkyl, an alkoxy, an alkylamino, a hydroxy, an oxy or an aryl, as defined herein.

“Phosphoric acid” refers to —P(O)(OR₅₁)OH wherein R₅₁ is a hydrogen atom, an alkyl group, an aryl group or an arylheterocyclic ring, as defined herein.

“Phosphinic acid” refers to —P(O)(R₅₁)OH wherein R₅₁ is a hydrogen atom, an alkyl group, an aryl group or an arylheterocyclic ring, as defined herein.

“Silyl” refers to —Si(R₇₃)(R₇₄)(R₇₅), wherein R₇₃, R₇₄ and R₇₅ are each independently a covalent bond, a lower alkyl, an alkoxy, an aryl or an arylalkoxy, as defined herein.

“Organic acid” refers to compound having at least one carbon atom and one or more functional groups capable of releasing a proton to a basic group. The organic acid preferably contains a carboxyl, a sulfonic acid or a phosphoric acid moeity. Exemplary organic acids include acetic acid, benzoic acid, citric acid, camphorsulfonic acid, methanesulfonic acid, taurocholic acid, chlordronic acid, glyphosphate, medronic acid, and the like.

“Inorganic acid” refers to a compound that does not contain at least one carbon atom and is capable of releasing a proton to a basic group. Exemplary inorganic acids include hydrochloric acid, sulfuric acid, nitric acid, phosphoric acid, and the like.

“Organic base” refers to a carbon containing compound having one or more functional groups capable of accepting a proton from an acid group. The organic base preferably contains an amine group. Exemplary organic bases include triethylamine, benzyldiethylamine, dimethylethyl amine, imidazole, pyridine, pipyridine, and the like.

“Organic nitric oxide enhancing salt” refers to any organic compound that contains a nitric oxide enhancing group and is capable of donating or transferring a biologically active form of nitrogen monoxide (i.e., nitric oxide) and can increase endogenous nitric oxide and also capable of ionically associating with a compound through at least one acidic group or basic group. Exemplary organic nitric oxide donor salts include N-[4-(hydroxymethyl)-1,2,5-oxadiazol-3-yl]carbonylglycine N-oxide (ACS registry number 158590-81-9), 3-[[5-oxido-4-(phenylsulfonyl)-1,2,5-oxadiazol-3-yl]oxy]methylpyridine (ACS registry number 174187-57-6), N,N-dimethyl-2-[[5-oxido-4-(phenylsulfonyl)-1,2,5-oxadiazol-3-yl]oxy]-ethanamine ((ACS registry number 186408-97-9), 2,2′,2″-nitrilotriethanol trinitrate (ACS registry number 7077-34-1), N,N-bis(2-hydroxyethyl)-nicotinamide dinitrate (ACS registry number 1157-74-0), [1-hydroxy-4-[[4-[(nitrooxy)methyl]benzoyl]amino] butylidene]bis-phosphonic acid (ACS registry number 636585-86-9), 4-(nitrooxy)-, (S)-(2-sulfoethyl) butanethioate (ACS registry number 586351-09-9), 3-(Nitryloxy)-2,2-bis[(nitryloxy)methyl] propionic acid (ACS registry number 67406-79-5), (S)-[2-[4-(2-hydroxyethyl)-1-piperidinyl]-1,1-dimethylethyl] thionitrite (ACS registry number 364590-39-6), (S)-[1,1-dimethyl-2-[(3-pyridinylcarbonyl)amino]ethyl] thionitrite (ACS registry number 307492-58-6), 2-(acetylamino)-2-carboxy-1,1-dimethylethyl thionitrite (ACS registry number 67776-06-1), and the like.

The NSAIDs that form the organic nitric oxide enhancing salt in accordance with the invention and/or are included in the compositions of the invention can be any of those known in the art, including those exemplified below.

Despite the introduction of many new drugs, aspirin (acetylsalicylic acid) is still the most widely prescribed antiinflammatory, analgesic and antipyretic compound and is a standard for the comparison and evaluation of all other NSAIDs. Salicylic acid itself is so irritating that it can only be used externally. However, derivatives, particularly salicylate esters and salts, have been prepared which provide ingestible forms of the salicylates which have the desired antiinflammatory and other properties. In addition to aspirin, which is the acetate ester of salicylic acid, are the difluorophenyl derivative (diflunisal) and salicylsalicylic acid (salsalate). Also available are the salts of salicylic acid, principally sodium salicylate. Sodium salicylate and aspirin are the two most commonly used preparations for systemic treatment. Other salicylates include salicylamide, sodium thiosalicylate, choline salicylate and magnesium salicylate. Also available are combinations of choline and magnesium salicylates. Also contemplated for use in the present invention are 5-aminosalicylic acid (mesalamine), salicylazosulfapyridine (sulfasalazine) and methylsalicylate.

Another group of NSAIDs are the pyrazolon derivatives, which include, for example, phenylbutazone, oxyphenbutazone, antipyrine, aminopyrine, dipyrone and apazone (azapropazone).

Another group of NSAIDs are the para-aminophenol derivatives, which are the so-called “coal tar” analgesics, including, for example, phenacetin and its active metabolite acetaminophen.

Another group of compounds for use in the present invention include indomethacin, a methylated indole derivative, and the structurally related compound sulindac.

Also contemplated is a group of compounds referred to as the fenamates which are derivatives of N-phenylanthranilic acid. The most well known of these compounds is mefenamic, meclofenamic, flufenamic, tolfenamic and etofenamic acids. They are used either as the acid or as pharmaceutically acceptable salts.

Another contemplated NSAID is tolmetin which, like the other NSAIDs discussed herein, causes gastric erosion and prolonged bleeding time.

Another group of NSAIDs are the propionic acid derivatives. Principal members of this group are, for example, ibuprofen, naproxen, flurbiprofen, fenoprofen and ketoprofen. Other members of this group, in use or study in countries outside the U.S., include, for example, fenbufen, pilprofen, oxaprozin, indoprofen and tiaprofenic acid.

Also contemplated for use in the invention is diclofenac, one of the series of phenylacetic acid derivatives that have been developed as antiinflammatory compounds. Other NSAIDs which are contemplated as suitable in the present invention include etodolac and nabumentone.

Each of the above NSAIDs is described more fully in the literature, such as in Goodman and Gilman, The Pharmacological Basis of Therapeutics (9th Edition), McGraw-Hill, 1995, Pgs. 617-657; the Merck Index on CD-ROM, 13^{th} Edition; and in U.S. Pat. Nos. 6,057,347 and 6,297,260 assigned to NitroMed. Inc., the disclosures of each of which are incorporated by reference herein in their entirety.

In one embodiment the NSAIDs must contain at least one carboxylic acid group (—COOH). The NSAIDs form salts with at least one organic nitric oxide enhancing compound that is ionically associated with the NSAID through one or more acid groups. The organic nitric oxide enhancing compounds that form salts with the NSAIDs are organic nitrates, organic nitrites, nitrosothiols, thionitrites, thionitrates, NONOates, heterocyclic nitric oxide donors and/or nitroxides that must contain a basic functionality, such as, for example an amidine group (—C(═NH)—NH₂), a guanidine group (—N(H)C(O)—NH₂) and/or a primary or secondary amine group (—NH), and the like. The heterocyclic nitric oxide donors are furoxans, sydnonimines, oxatriazole-5-ones and/or oxatriazole-5-imines.

In another embodiment, the invention describes NSAIDs of Formula (I):

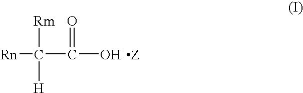

wherein:
Rₘ is a hydrogen or a lower alkyl group;
Rₙ is:

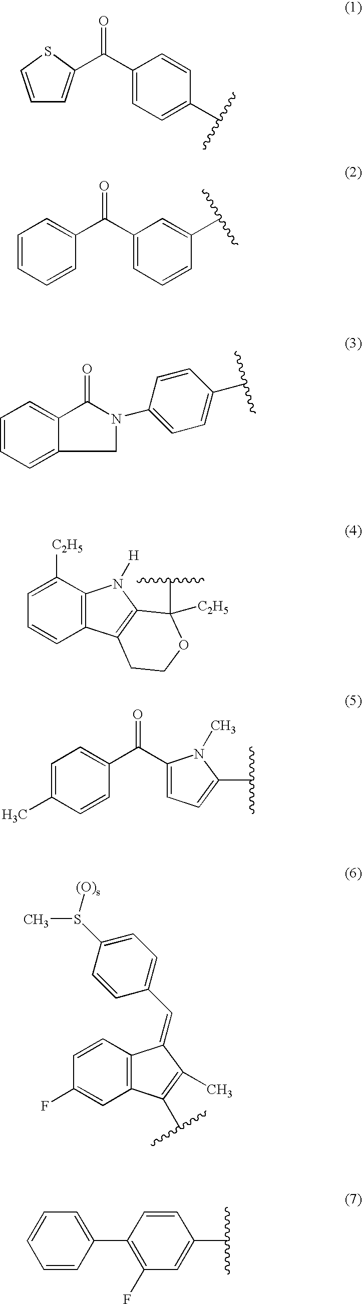

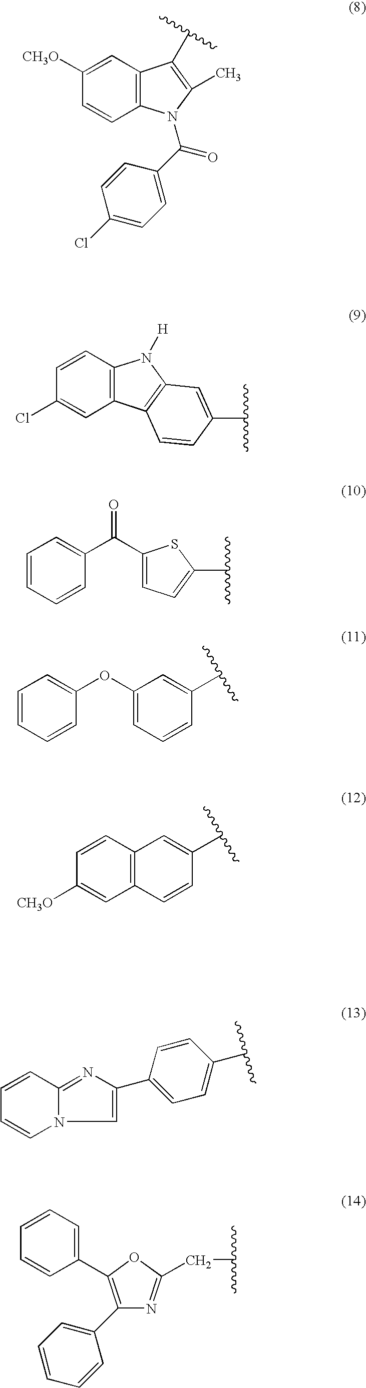

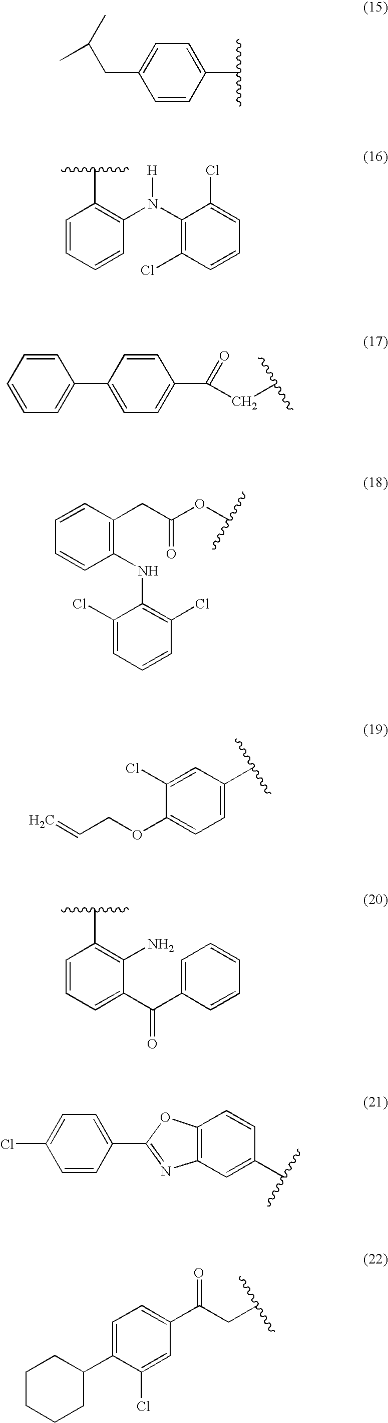

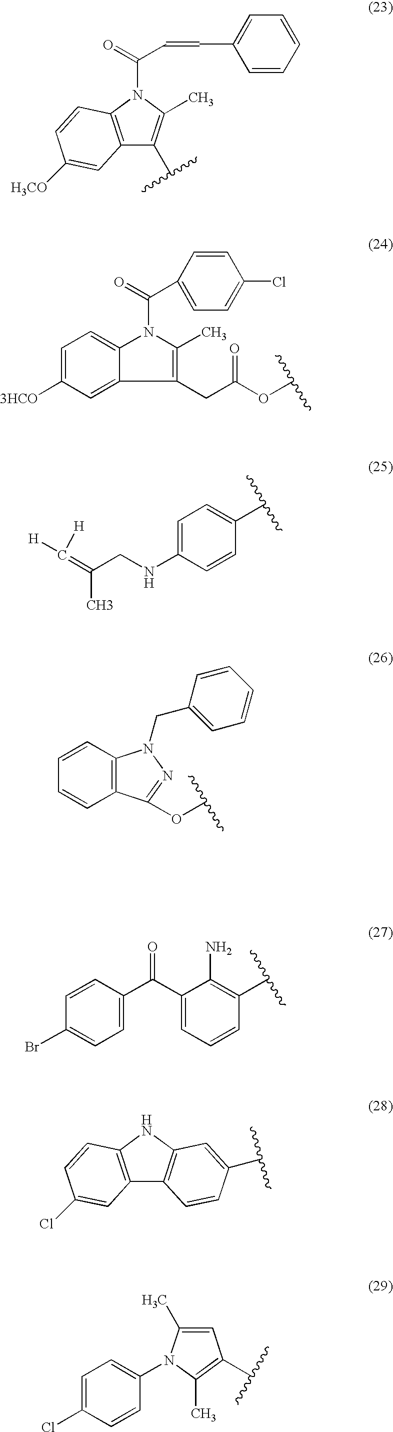

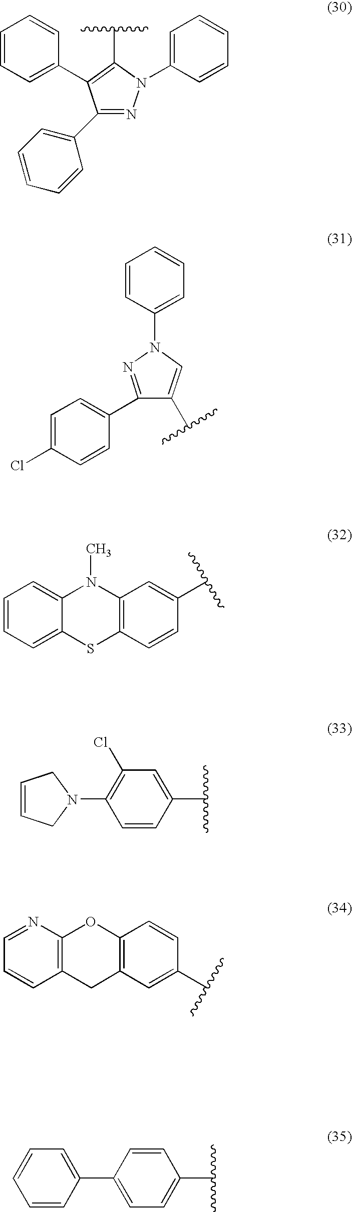

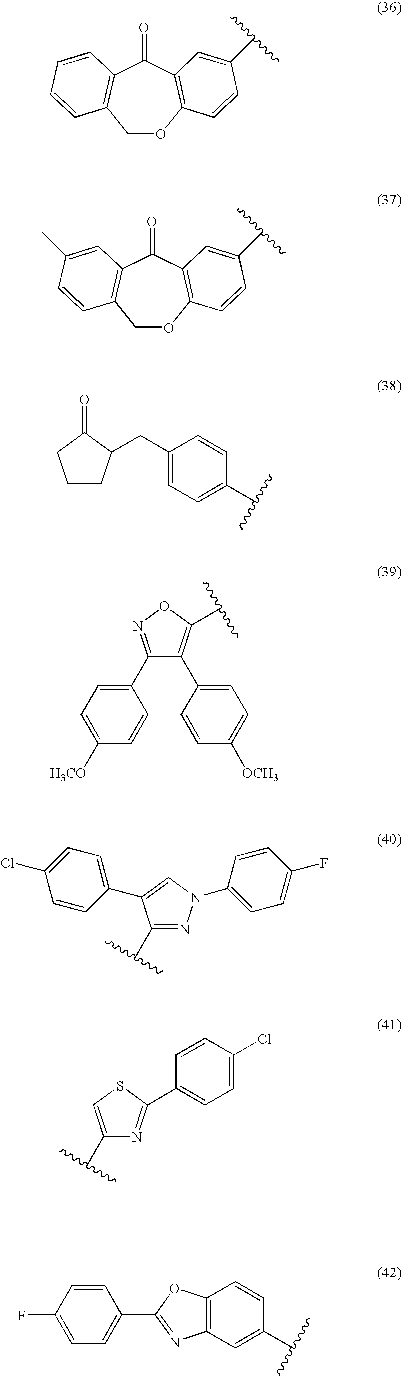

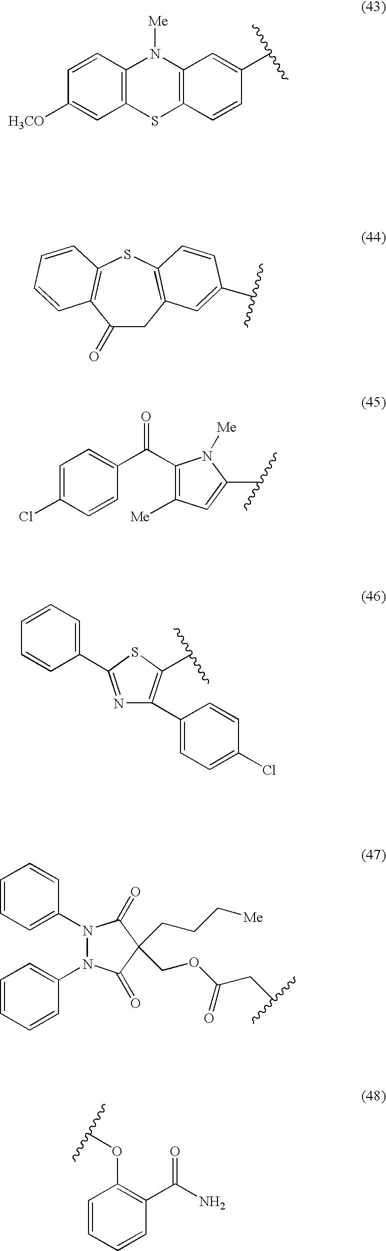

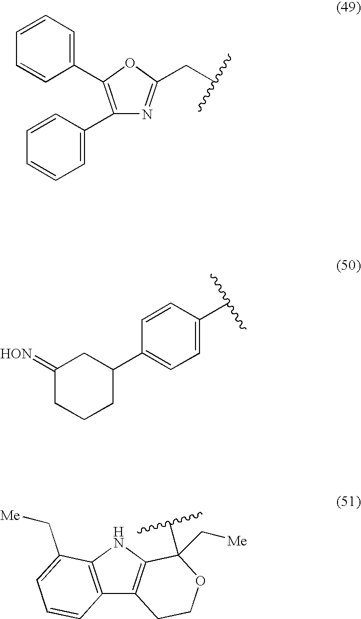

s is an integer of 0 or 1;

Z is an organic base or —N(R₃₈)(R₃₉)(R₄₀);

R₃₈, R₃₉ and R₄₀ are each independently selected from K or Rₑ, or R₃₈ and R₃₉ taken together with the nitrogen to which they are attached are a heterocyclic ring, with the proviso that when the heterocyclic ring is an aromatic ring it can be substituted at any position by L and R₃₉ is not present;

L is —(W₃)ₐ-E_{b}-(C(Rₑ)(R_{f}))ₚ₁-E_{c}-(C(Rₑ)(R_{f}))ₓ—(W₃)_{d}—(C(Rₑ)(R_{f}))_{y}—(W₃)ᵢ-Eⱼ-(W₃)_{g}—(C(Rₑ)(R_{f}))_{z}—V₄;

K is —(W₃)ₐ-E_{b}-(C(Rₑ)(R_{f}))ₚ₁-E_{c}-(C(Rₑ)(R_{f}))—(W₃)_{d}—(C(Rₑ)(R_{f}))_{y}—(W₃)ᵢ-Eⱼ-(W₃)_{g}—(C(Rₑ)(R_{f}))_{z}—V₄;

a, b, c, d, g, i and j are each independently an integer from 0 to 3;

p₁, x, y and z are each independently an integer from 0 to 10;

V₄ is V₃, Rₑ, —U₃—V₅ or V₆;

V₃ is:

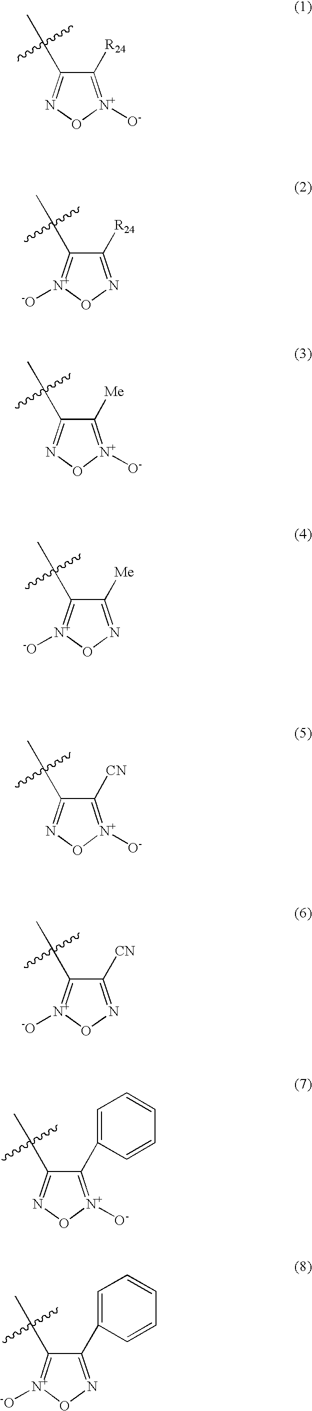

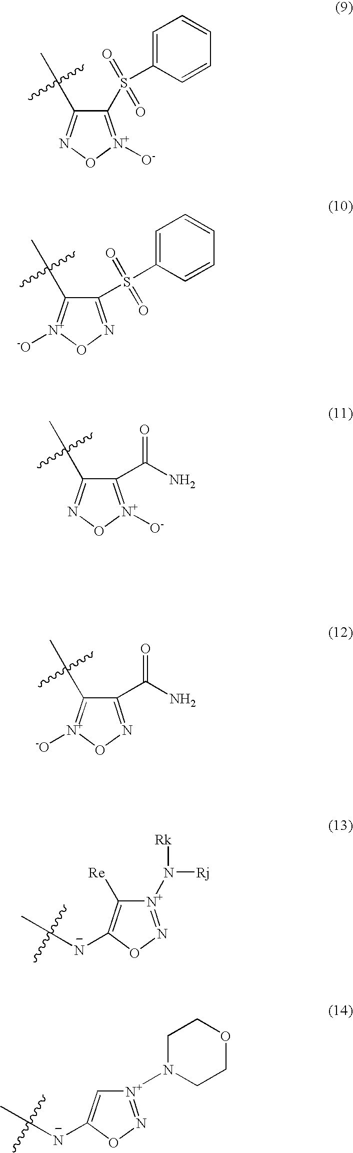

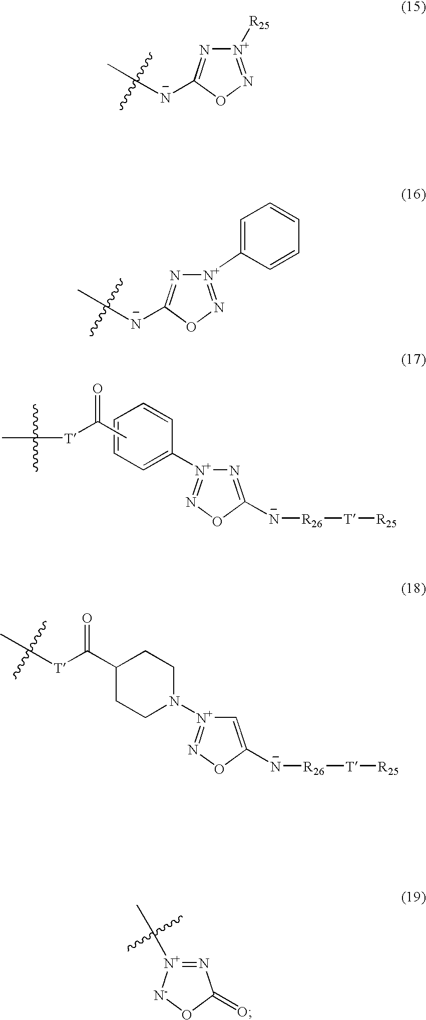

R₂₄ is —C₆H₄R₃₇, —CN, —S(O)₂—C₆H₄R₃₇, —C(O)—N(Rₐ)(Rᵢ), —NO₂, —C(O)—OR₂₅ or —S(O)₂—R₂₅;

R₂₅ is an aryl group, a lower alkyl group, a haloalkyl group, a hydroxyalkyl group or an arylalkyl group;

R₂₆ is —C(O)— or —S(O)₂—;

R₃₇ is a hydrogen, —CN, —S(O)₂—R₂₅, —C(O)—N(Rₐ)(Rᵢ), —NO₂ or —C(O)—OR₂₅;

T′ is oxygen, sulfur or NR₁₆;

R₁₆ is a hydrogen, a lower alkyl group, or an aryl group;

V₆ is:

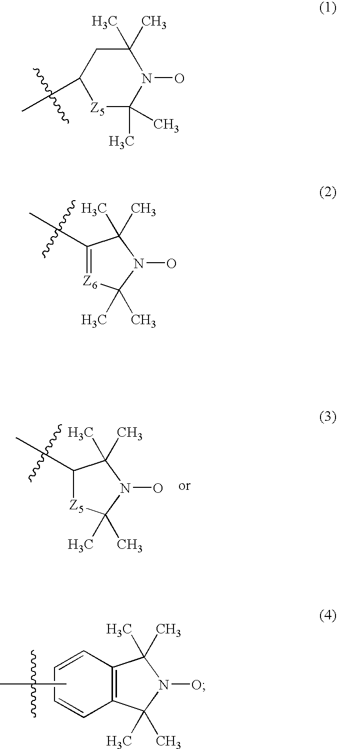

Z₅ is —CH₂ or oxygen;

Z₆ is —CH or nitrogen;

W₃ at each occurrence is independently —C(O)—, —C(S)—, -T₃-, —(C(Rₑ)(R_{f}))ₕ—, —N(Rₐ)Rᵢ, an alkyl group, an aryl group, a heterocyclic ring, an arylheterocyclic ring, —(CH₂CH₂O)_{q1}— or a heterocyclic nitric oxide donor;

E at each occurrence is independently -T₃-, an alkyl group, an aryl group, —(C(Rₑ)(R_{f}))ₕ—, a heterocyclic ring, an arylheterocyclic ring, —(CH₂CH₂O)_{q1}— or Y₄;

Y₄ is:

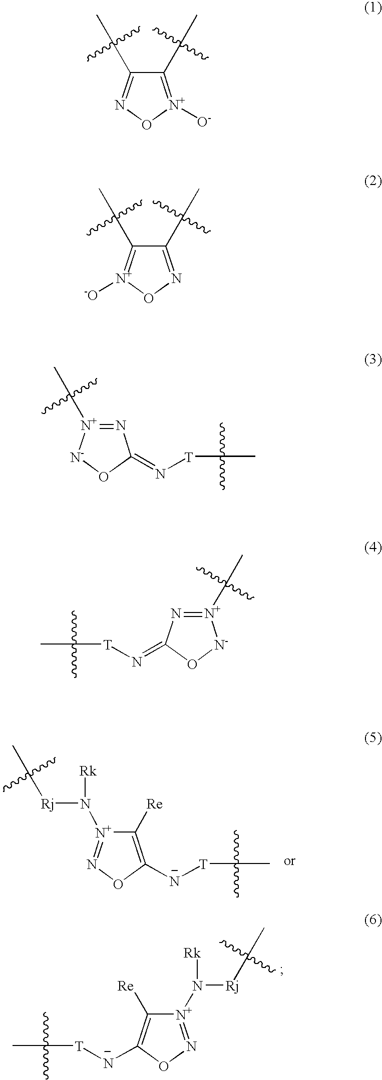

T is a —S(O)ₒ—; a carbonyl or a covalent bond;

o is an integer from 0 to 2;

Rⱼ and Rₖ are independently selected from an alkyl group, an aryl group, or Rⱼ and Rₖ taken together with the nitrogen atom to which they are attached are a heterocylic ring;

T₃ at each occurrence is independently a covalent bond, a carbonyl, an oxygen, —S(O)ₒ— or —N(Rₐ)Rᵢ;

h is an integer form 1 to 10;

q₁ is an integer from 1 to 5;

Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano, an aminoalkyl, an aminoaryl, an aryl, an arylalkyl, an alkylaryl, a carboxamido, an alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, —U₃—V₅, V₆, —(C(Rₒ)(Rₚ))ₖ₁—U₃—V₅, —(C(Rₒ)(Rₚ))ₖ₁—U₃—V₃, —(C(Rₒ)(Rₚ))ₖ₁—U₃—V₆, —(C(Rₒ)(Rₚ))ₖ₁—U₃—C(O)—V₆, or Rₑ and R_{f} taken together with the carbons to which they are attached form a carbonyl, a methanthial, a heterocyclic ring, a cycloalkyl group, an aryl group, an oxime, an imine, a hydrazone, a bridged cycloalkyl group,

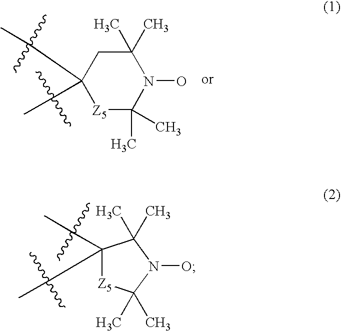

Rₒ and Rₚ are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, an alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, —U₃—V₅, V₆, or Rₒ and Rₚ taken together with the carbons to which they are attached form a carbonyl, a methanthial, a heterocyclic ring, a cycloalkyl group, an aryl group, an oxime, an imine, a hydrazone a bridged cycloalkyl group,

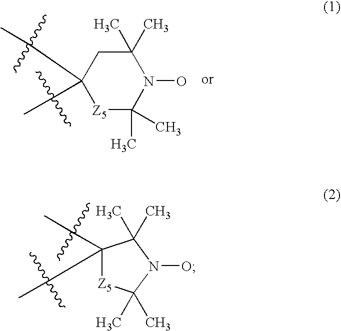

U₃ is an oxygen, sulfur or —N(Rₐ)Rᵢ;

V₅ is —NO or —NO₂ (i.e. an oxidized nitrogen);

k₁ is an integer from 1 to 3;

Rₐ is a lone pair of electrons, a hydrogen or an alkyl group;

Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, an arylsulphonyloxy, a sulfonamido, a carboxamido, a carboxylic ester, an aminoalkyl, an aminoaryl, —CH₂—C—(U₃—V₅)(Rₑ)(R_{f}), a bond to an adjacent atom creating a double bond to that atom or —(N₂O₂—).M₁⁺, wherein M₁⁺ is an organic or inorganic cation; and

with the proviso that the compound of Formula (I) must contain at least one organic nitric oxide enhancing compound linked via a salt bridge (i.e., .) to at least one carboxylic acid group.

In cases where multiple designations of variables which reside in sequence are chosen as a “covalent bond” or the integer chosen is 0, the intent is to denote a single covalent bond connecting one radical to another. For example, E₀ would denote a covalent bond, while E₂ denotes (E-E) and (C(R₄)(R₄))₂ denotes —C(R₄)(R₄)—C(R₄)(R₄)—.

In another embodiment, the invention describes NSAIDs of Formula (II):

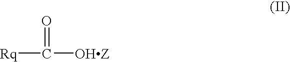

wherein:

R_{q} is:

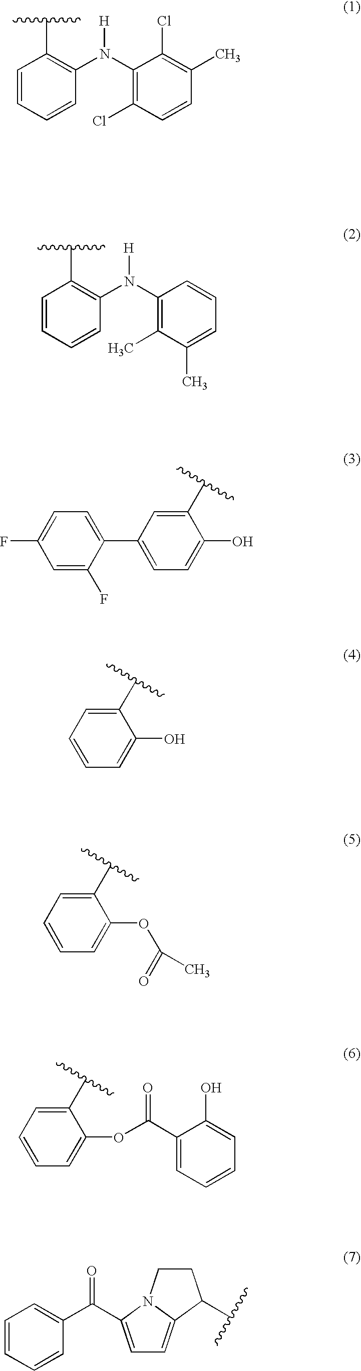

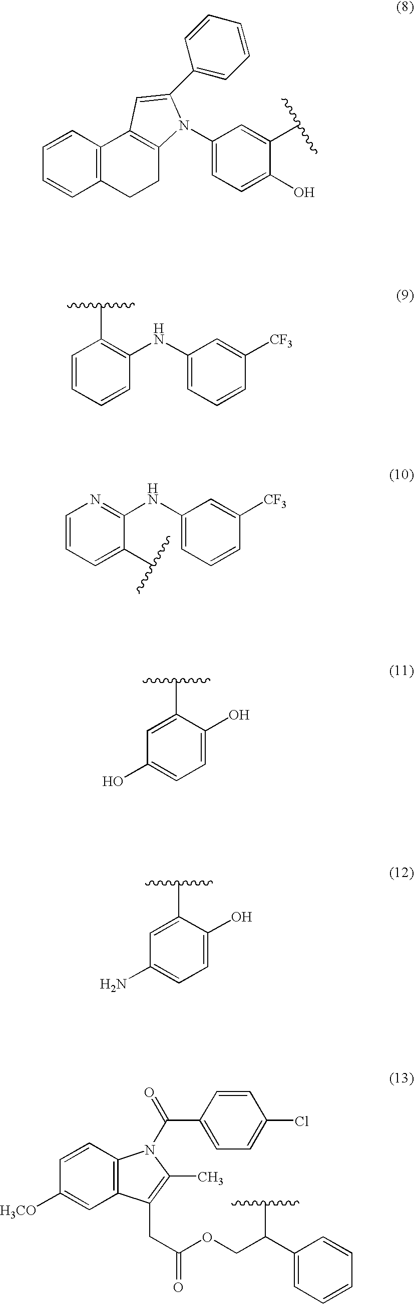

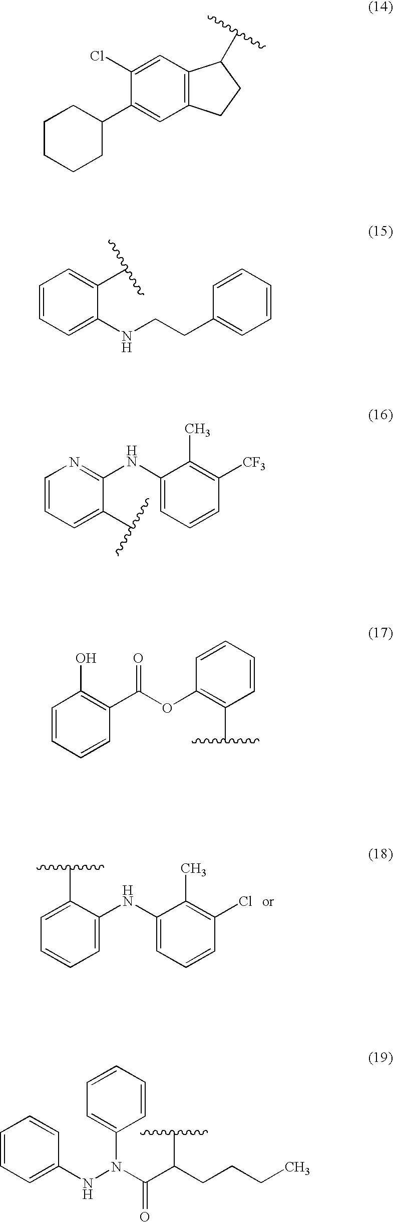

and Z is as defined herein; and

with the proviso that the compounds of Formula (II) must contain at least one organic nitric oxide enhancing compound linked via a salt bridge (i.e., .) to at least one carboxylic acid group.

In other embodiments of the invention, the organic nitric oxide enhancing salt of the NSAID of Formula (I) is an organic nitric oxide enhancing salt of acemetacin, an organic nitric oxide enhancing salt of aceclofenac, an organic nitric oxide enhancing salt of alclofenac, an organic nitric oxide enhancing salt of alminoprofen, an organic nitric oxide enhancing salt of amfenac, an organic nitric oxide enhancing salt of bendazac, an organic nitric oxide enhancing salt of benoxaprofen, an organic nitric oxide enhancing salt of bromfenac, an organic nitric oxide enhancing salt of bucloxic acid, an organic nitric oxide enhancing salt of butibufen, an organic nitric oxide enhancing salt of carprofen, an organic nitric oxide enhancing salt of cinmetacin, an organic nitric oxide enhancing salt of clopirac, an organic nitric oxide enhancing salt of diclofenac, an organic nitric oxide enhancing salt of etodolac, an organic nitric oxide enhancing salt of felbinac, an organic nitric oxide enhancing salt of fenclozic acid, an organic nitric oxide enhancing salt of fenbufen, an organic nitric oxide enhancing salt of fenoprofen, an organic nitric oxide enhancing salt of fentiazac, an organic nitric oxide enhancing salt of flunoxaprofen, an organic nitric oxide enhancing salt of flurbiprofen, an organic nitric oxide enhancing salt of ibufenac, an organic nitric oxide enhancing salt of ibuprofen, an organic nitric oxide enhancing salt of indomethacin, an organic nitric oxide enhancing salt of isofezolac, an organic nitric oxide enhancing salt of isoxepac, an organic nitric oxide enhancing salt of indoprofen, an organic nitric oxide enhancing salt of ketoprofen, an organic nitric oxide enhancing salt of lonazolac, an organic nitric oxide enhancing salt of loxoprofen, an organic nitric oxide enhancing salt of metiazinic acid, an organic nitric oxide enhancing salt of mofezolac, an organic nitric oxide enhancing salt of miroprofen, an organic nitric oxide enhancing salt of naproxen, an organic nitric oxide enhancing salt of oxaprozin, an organic nitric oxide enhancing salt of pirozolac, an organic nitric oxide enhancing salt of pirprofen, an organic nitric oxide enhancing salt of pranoprofen, an organic nitric oxide enhancing salt of protizinic acid, an organic nitric oxide enhancing salt of salicylamide O-acetic acid, an organic nitric oxide enhancing salt of sulindac, an organic nitric oxide enhancing salt of suprofen, an organic nitric oxide enhancing salt of suxibuzone, an organic nitric oxide enhancing salt of tiaprofenic acid, an organic nitric oxide enhancing salt of tolmetin, an organic nitric oxide enhancing salt of xenbucin, an organic nitric oxide enhancing salt of ximoprofen, an organic nitric oxide enhancing salt of zaltoprofen or an organic nitric oxide enhancing salt of zomepirac; and the organic nitric oxide enhancing salt of NSAID of Formula II is an organic nitric oxide enhancing salt of aspirin, an organic nitric oxide enhancing salt of acemetcin, an organic nitric oxide enhancing salt of bumadizon, an organic nitric oxide enhancing salt of carprofenac, an organic nitric oxide enhancing salt of clidanac, an organic nitric oxide enhancing salt of diflunisal, an organic nitric oxide enhancing salt of enfenamic acid, an organic nitric oxide enhancing salt of fendosal, an organic nitric oxide enhancing salt of flufenamic acid, an organic nitric oxide enhancing salt of flunixin, an organic nitric oxide enhancing salt of gentisic acid, an organic nitric oxide enhancing salt of ketorolac, an organic nitric oxide enhancing salt of meclofenamic acid, an organic nitric oxide enhancing salt of mefenamic acid, an organic nitric oxide enhancing salt of mesalamine, an organic nitric oxide enhancing salt of niflumic acid, an organic nitric oxide enhancing salt of salsalate, an organic nitric oxide enhancing salt of tolfenamic acid or an organic nitric oxide enhancing salt of tropensin.

In other embodiments of the invention, the organic nitric oxide donor salt of the NSAID of Formula (I) is an organic nitric oxide enhancing salt of acemetacin, an organic nitric oxide enhancing salt of aceclofenac of Formula (III), an organic nitric oxide enhancing salt of alclofenac of Formula (IV), an organic nitric oxide enhancing salt of bromfenac of Formula (V), an organic nitric oxide enhancing salt of carprofen of Formula (VI), an organic nitric oxide enhancing salt of diclofenac of Formula (VII), an organic nitric oxide enhancing salt of etodolac of Formula (VIII), an organic nitric oxide enhancing salt of fenbufen of Formula (IX), an organic nitric oxide enhancing salt of fenoprofen of Formula (X), an organic nitric oxide enhancing salt of flurbiprofen of Formula (XI), an organic nitric oxide enhancing salt of ibuprofen of Formula (XII), an organic nitric oxide enhancing salt of indomethacin of Formula (XIII), an organic nitric oxide enhancing salt of ketoprofen of Formula (XIV), an organic nitric oxide enhancing salt of loxoprofen of Formula (XV), an organic nitric oxide enhancing salt of naproxen of Formula (XVI), an organic nitric oxide enhancing salt of oxaprozin of Formula (XVII), an organic nitric oxide enhancing salt of pirozolac of Formula (XVIII), an organic nitric oxide enhancing salt of sulindac of Formula (XIX), an organic nitric oxide enhancing salt of suprofen of Formula (XX), an organic nitric oxide enhancing salt of tolmetin of Formula (XXI); and the organic nitric oxide enhancing salt of NSAID of Formula II is an organic nitric oxide enhancing salt of aspirin of Formula (XXII), an organic nitric oxide enhancing salt of acemetcin of Formula (XXIII), an organic nitric oxide enhancing salt of diflunisal of Formula (XXIV), an organic nitric oxide enhancing salt of enfenamic acid of Formula (XXV), an organic nitric oxide enhancing salt of flufenamic acid of Formula (XXVI), an organic nitric oxide enhancing salt of gentisic acid of Formula (XXVII), an organic nitric oxide enhancing salt of ketorolac of Formula (XXVIII), an organic nitric oxide enhancing salt of meclofenamic acid of Formula (XXIX), an organic nitric oxide enhancing salt of mefenamic acid of Formula (XXX), an organic nitric oxide enhancing salt of mesalamine of Formula (XXXI), an organic nitric oxide enhancing salt of salsalate of Formula (XXXII) or an organic nitric oxide enhancing salt of tolfenamic acid of Formula (XXXIII);

and the compound of Formula (III) is:

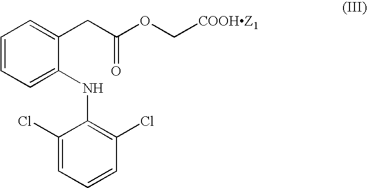

and the compound of Formula (IV) is:

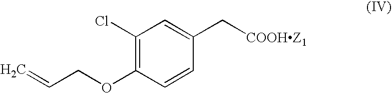

and the compound of Formula (V) is:

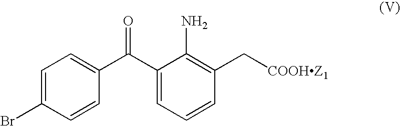

and the compound of Formula (VI) is:

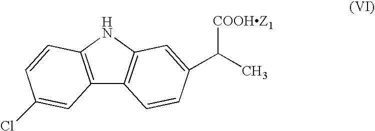

and the compound of Formula (VII) is:

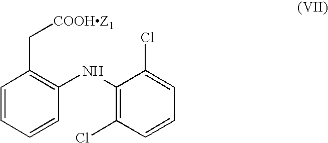

and the compound of Formula (VIII) is:

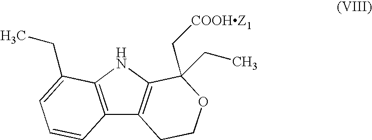

and the compound of Formula (IX) is:

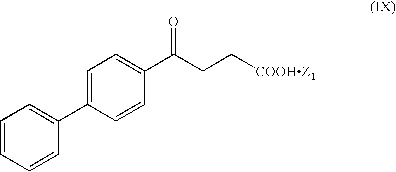

and the compound of Formula (X) is:

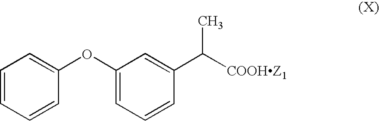

and the compound of Formula (XI) is:

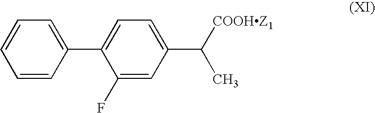

and the compound of Formula (XII) is:

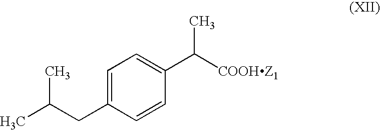

and the compound of Formula (XIII) is:

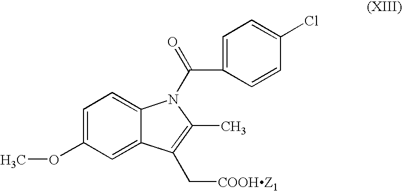

and the compound of Formula (XIV) is:

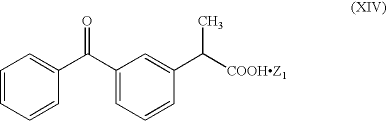

and the compound of Formula (XV) is:

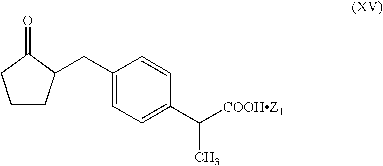

and the compound of Formula (XVI) is:

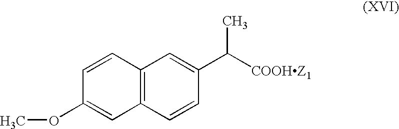

and the compound of Formula (XVII) is:

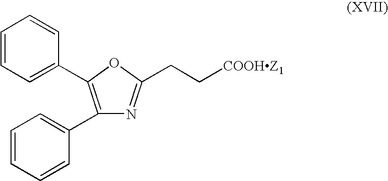

and the compound of Formula (XVIII) is:

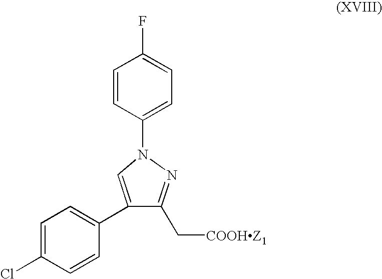

and the compound of Formula (XIX) is:

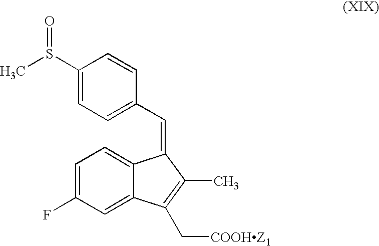

and the compound of Formula (XX) is:

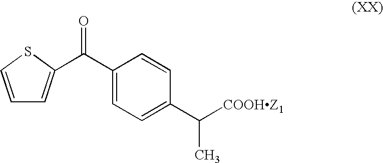

and the compound of Formula (XXI) is:

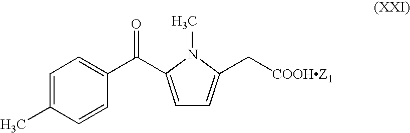

and the compound of Formula (XXII) is:

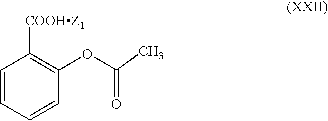

and the compound of Formula (XXIII) is:

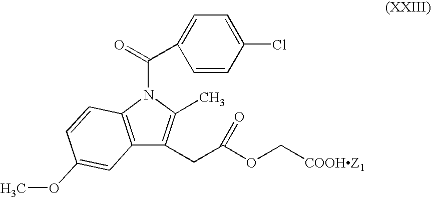

and the compound of Formula (XXIV) is:

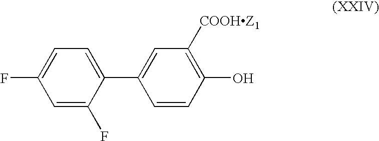

and the compound of Formula (XXV) is:

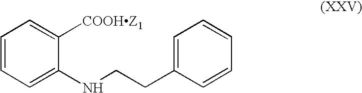

and the compound of Formula (XXVI) is:

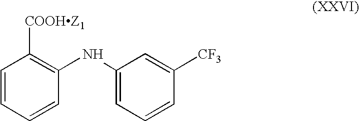

and the compound of Formula (XXVII) is:

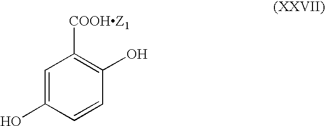

and the compound of Formula (XXVIII) is:

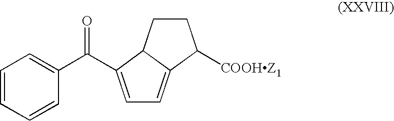

and the compound of Formula (XXIX) is:

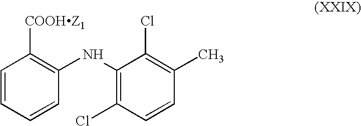

and the compound of Formula (XXX) is:

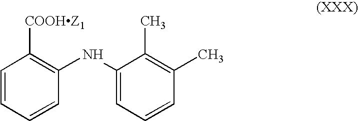

and the compound of Formula (XXXI) is:

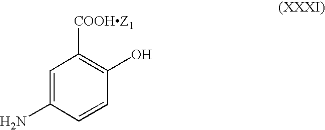

and the compound of Formula (XXXII) is:

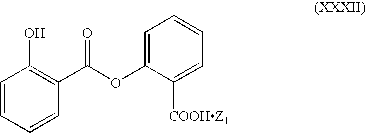

and the compound of Formula (XXXIII) is:

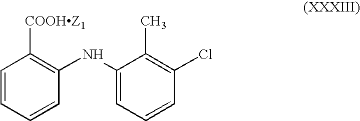

Z₁ is:

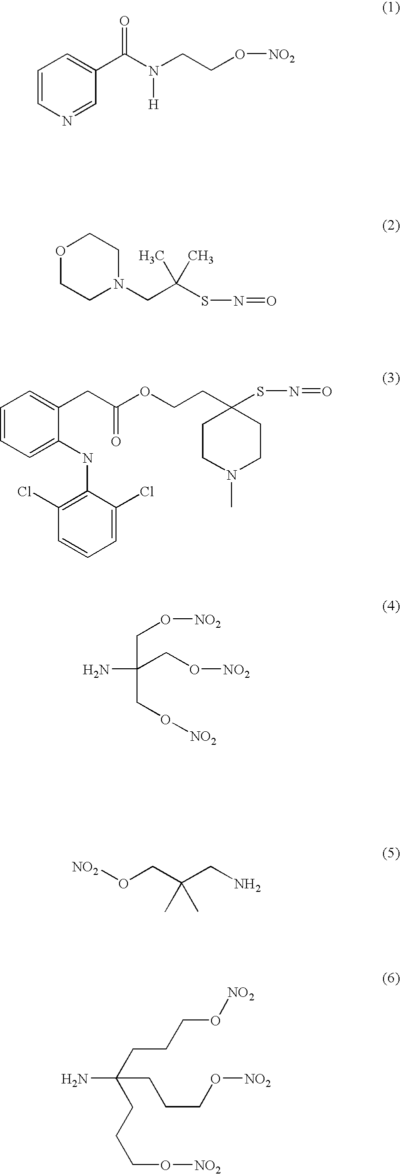

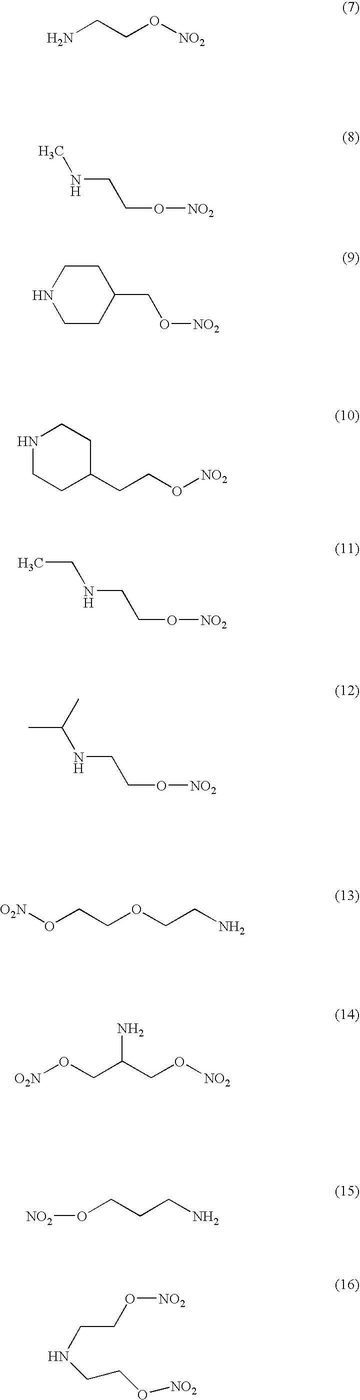

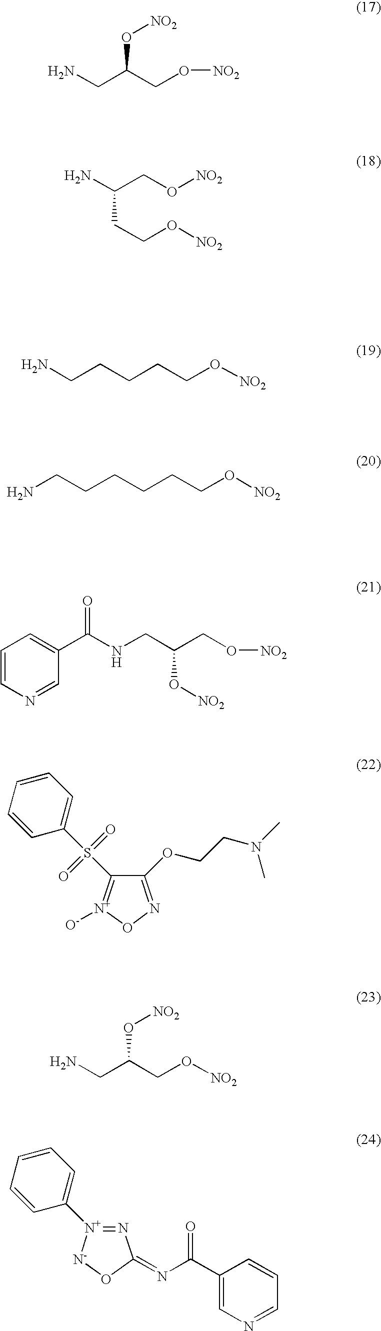

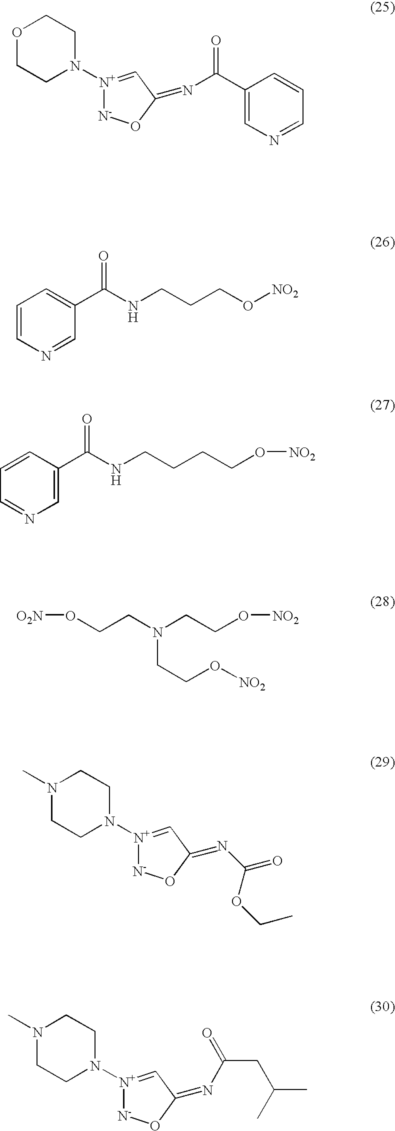

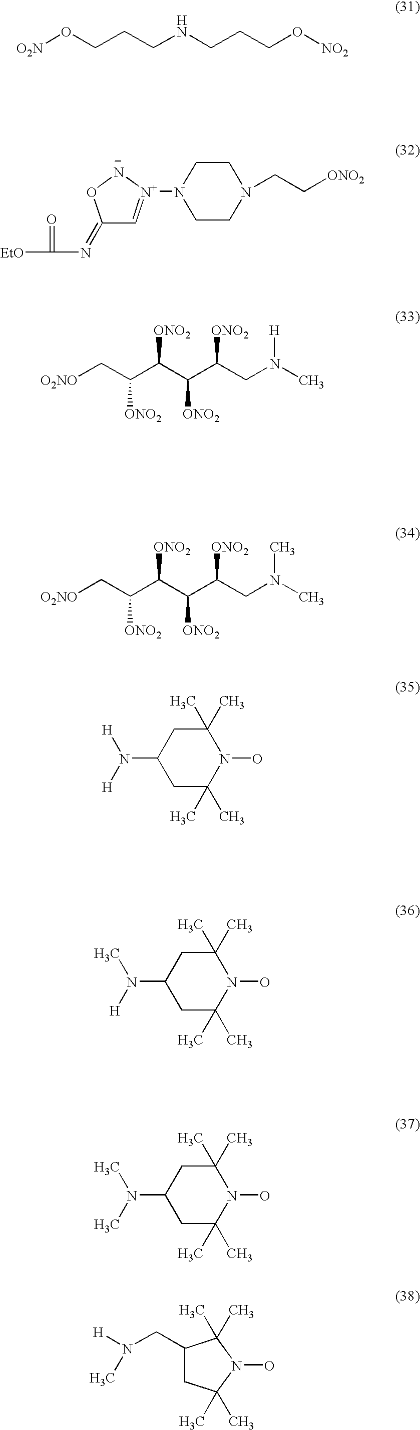

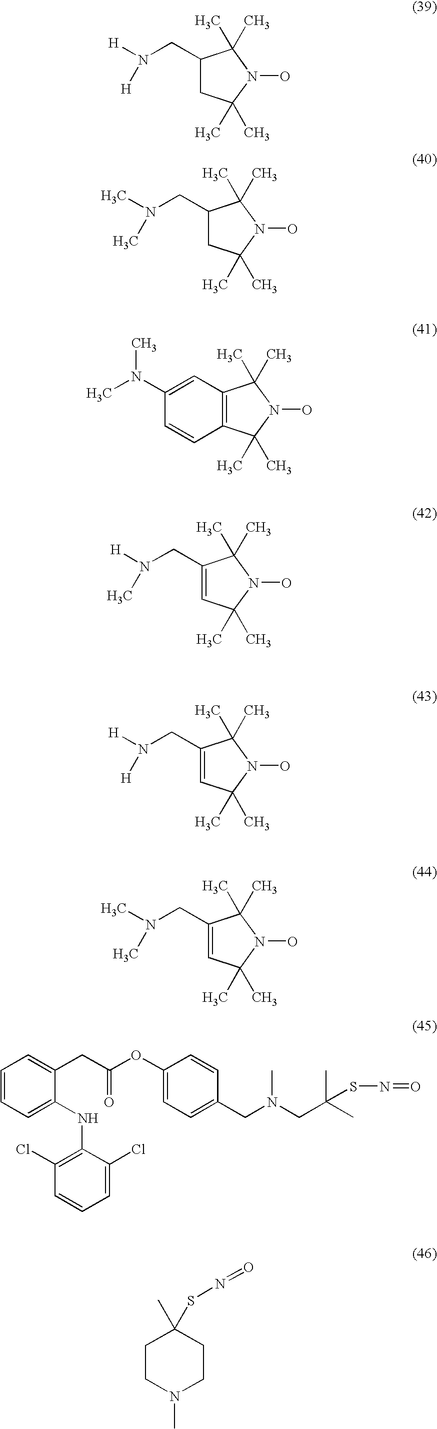

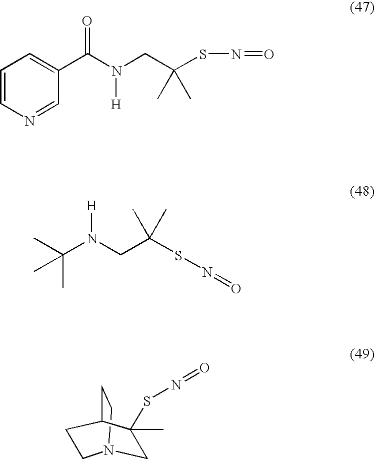

with the proviso that the compounds of Formula (III) and (XXXIII) must contain at least one organic nitric oxide enhancing compound linked via a salt bridge (i.e., . or ▪) to at least one carboxylic acid group in the compounds of Formula (III) and (XXXIII).

In other embodiments, the organic nitric oxide enhancing compounds that form salts are organic nitrates, organic nitrites, nitrosothiols, thionitrites, thionitrates, NONOates, heterocyclic nitric oxide donors and nitroxides.

In one embodiment, the organic nitric oxide enhancing salts of NSAIDs do not contain at least one nitrate ion mole per mole of the NSAID.

Compounds of the invention that have one or more asymmetric carbon atoms may exist as the optically pure enantiomers, pure diastereomers, mixtures of enantiomers, mixtures of diastereomers, racemic mixtures of enantiomers, diastereomeric racemates or mixtures of diastereomeric racemates. It is to be understood that the invention anticipates and includes within its scope all such isomers and mixtures thereof.

Another embodiment of the invention provides the organic nitric oxide enhancing salts of the metabolites of the NSAID compounds. These metabolites include, but are not limited to, degradation products, hydrolysis products, and the like, of the NSAID compounds.

Another embodiment of the invention provides processes for making the novel salts of the invention. The reactions are performed in solvents appropriate to the reagents and materials used are suitable for the transformations being effected. It is understood by one skilled in the art of organic synthesis that the functionality present in the molecule must be consistent with the chemical transformation proposed. This will, on occasion, necessitate judgment by the routineer as to the order of synthetic steps, protecting groups required, and deprotection conditions. Substituents on the starting materials may be incompatible with some of the reaction conditions required in some of the methods described, but alternative methods and substituents compatible with the reaction conditions will be readily apparent to one skilled in the art. The use of sulfur and oxygen protecting groups is well known for protecting thiol and alcohol groups against undesirable reactions during a synthetic procedure and many such protecting groups are known and described by, for example, Greene and Wuts, Protective Groups in Organic Synthesis, Third Edition, John Wiley & Sons, New York (1999).

The chemical reactions described herein are generally disclosed in terms of their broadest application to the preparation of the compounds of this invention. Occasionally, the reactions may not be applicable as described to each compound included within the disclosed scope. The compounds for which this occurs will be readily recognized by one skilled in the art. In all such cases, either the reactions can be successfully performed by conventional modifications known to one skilled in the art, e.g., by appropriate protection of interfering groups, by changing to alternative conventional reagents, by routine modification of reaction conditions, and the like, or other reactions disclosed herein or otherwise conventional, will be applicable to the preparation of the corresponding compounds of this invention. In all preparative methods, all starting materials are known or readily prepared from known starting materials.

The salts of the invention are formulated according to well known techniques in the prior art, see for example, Remington's Pharmaceutical Sciences.

The NSAIDs are either commercially available or can be prepared according to the methods described are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

The novel organic nitric oxide enhancing compounds can be synthesized by one skilled in the art using conventional methods. Known methods for linking a nitric oxide enhancing group to compounds, such as, for example, linking nitrates, thionitrates, nitrites, thionitrites, (i.e. nitrosated and/or nitrosylated compounds), NONOates, heterocyclic nitric oxide donors, and the like are described in the literature. For example, heterocyclic nitric oxide donor compounds are described in WO 99/64417, WO 94/01422; EP 0 574 726 A1, EP 0 683 159 A1; and in J. Med. Chem., 47: 2688-2693 (2004); J. Med. Chem., 47: 1840-1846 (2004); J. Med. Chem., 46: 3762-3765 (2003); J. Med. Chem., 46: 747-754 (2003); Chem Rev., 102: 1091-1134 (2002); J. Med. Chem., 42: 1941-1950 (1999); J. Med. Chem., 41: 5393-5401 (1998); J. Med. Chem., 38: 4944-4949 (1995); Arzneim. Forsch. Drug Res., 47 (II): 847-854 (1997); the disclosures of each of which are incorporated by reference herein in their entirety. The methods of linking the heterocyclic nitric oxide donor group to compounds described in these references can be applied by one skilled in the art to produce any of the organic nitric oxide enhancing compounds described herein. Linking a nitrate group, a thionitrate group, a nitrite group and/or a thionitrite group to a compound can be achieved by the nitrosated and/or nitrosylated of a compound through one or more sites such as oxygen, sulfur and/or nitrogen using conventional methods known to one skilled in the art. Known methods for nitrosating and/or nitrosylating compounds are described in U.S. Pat. Nos. 5,380,758, 5,859,053, 5,703,073 and 6,297,260; and in WO 94/03421, WO 94/04484, WO 94/12463, WO 95/09831, WO 95/19952, WO 95/30641, WO 97/27749, WO 98/09948, WO 98/19672, WO 98/21193, WO 00/51988, WO 00/61604, WO 00/72838, WO 01/00563, WO 01/04082, WO 01/10814, WO 01/12584, WO 01/45703, WO 00/61541, WO 00/61537, WO 02/11707, WO 02/30866 and in Oae et al, Org. Prep. Proc. Int., 15(3):165-198 (1983), the disclosures of each of which are incorporated by reference herein in their entirety. The methods of nitrosating and/or nitrosylating the compounds described in these references can be applied by one skilled in the art to produce any of the nitrosated and/or nitrosylated compounds described herein.

Known methods of linking the nitroxide group to compounds are described in U.S. Pat. Nos. 6,448,267, 6,455,542, 6,759,430, and in WO 2004/050084, WO 03/088961, the disclosures of each of which are incorporated by reference herein in their entirety.

The organic nitric oxide enhancing salts of the NSAIDs are prepared by the following methods. When the NSAID to be salified is available as free base soluble in an organic solvent, which preferably does not contain hydroxyl groups, for example acetonitrile, ethyl acetate, tetrahydrofuran, and the like, the salt is prepared by dissolving the compound in the solvent at a concentration preferably equal to or higher than 10% w/v, adding the amount of organic nitric oxide enhancing compound corresponding to the moles of the ionizable groups in the NSAID. The organic nitric oxide enhancing compound is preferably diluted in the same solvent. The salt is generally recovered by filtration and washed with the solvent. When the NSAID is not very soluble, or is available in the form of a not very soluble salt in the above mentioned solvents, a hydroxylated solvent, such as, for examples, methyl alcohol, ethyl alcohol, water, and the like, can be used.

When the starting NSAID is an inorganic salt, the corresponding base can also be prepared by treatment with a saturated solution of sodium or potassium bicarbonate or carbonate, or with a diluted solution of sodium or potassium hydroxide. The base is then extracted with a suitable organic solvent (for example halogenated solvents, esters, ethers), which is then dried. The organic solution is evaporated and the organic nitric oxide enhancing salt is prepared as described herein.

Compounds contemplated for use in the invention, e.g., organic nitric oxide enhancing salts of NSAIDs, are, optionally, used in combination with nitric oxide enhancing compounds that release nitric oxide, increase endogeneous levels of nitric oxide or otherwise directly or indirectly deliver or transfer a biologically active form of nitrogen monoxide to a site of its intended activity, such as on a cell membrane in vivo.

Nitrogen monoxide can exist in three forms: NO— (nitroxyl), NO. (nitric oxide) and NO⁺ (nitrosonium). NO. is a highly reactive short-lived species that is potentially toxic to cells. This is critical because the pharmacological efficacy of NO depends upon the form in which it is delivered. In contrast to the nitric oxide radical (NO.), nitrosonium (NO⁺) does not react with O₂ or O₂— species, and functionalities capable of transferring and/or releasing NO⁺ and NO— are also resistant to decomposition in the presence of many redox metals. Consequently, administration of charged NO equivalents (positive and/or negative) does not result in the generation of toxic by-products or the elimination of the active NO group.

The term “nitric oxide” encompasses uncharged nitric oxide (NO.) and charged nitrogen monoxide species, preferably charged nitrogen monoxide species, such as nitrosonium ion (NO⁺) and nitroxyl ion (NO—). The reactive form of nitric oxide can be provided by gaseous nitric oxide. The nitrogen monoxide releasing, delivering or transferring compounds have the structure F—NO, wherein F is a nitrogen monoxide releasing, delivering or transferring group, and include any and all such compounds which provide nitrogen monoxide to its intended site of action in a form active for its intended purpose.

The term “NO adducts” encompasses any nitrogen monoxide releasing, delivering or transferring compounds, including, for example, S-nitrosothiols, nitrites, nitrates, S-nitrothiols, sydnonimines, 2-hydroxy-2-nitrosohydrazines, (NONOates), (E)-alkyl-2-((E)-hydroxyimino)-5-nitro-3-hexeneamide (FK-409), (E)-alkyl-2-((E)-hydroxyimino)-5-nitro-3-hexeneamines, N-((2Z,3E)-4-ethyl-2-(hydroxyimino)-6-methyl-5-nitro-3-heptenyl)-3-pyridinecarboxamide (FR 146801), N-nitrosoamines, N-hydroxyl nitrosamines, nitrosimines, diazetine dioxides, oxatriazole 5-imines, oximes, hydroxylamines, N-hydroxyguanidines, hydroxyureas, benzofuroxanes, furoxans as well as substrates for the endogenous enzymes which synthesize nitric oxide.

Suitable NONOates include, but are not limited to, (Z)-1-(N-methyl-N-(6-(N-methyl-ammoniohexyl)amino))diazen-1-ium-1,2-diolate (“MAHMA/NO”), (Z)-1-(N-(3-ammoniopropyl)-N-(n-propyl)amino)diazen-1-ium-1,2-diolate (“PAPA/NO”), (Z)-1-(N-(3-aminopropyl)-N-(4-(3-aminopropylammonio)butyl)-amino) diazen-1-ium-1,2-diolate (spermine NONOate or “SPER/NO”) and sodium(Z)-1-(N,N-diethylamino)diazenium-1,2-diolate (diethylamine NONOate or “DEA/NO”) and derivatives thereof. NONOates are also described in U.S. Pat. Nos. 6,232,336, 5,910,316 and 5,650,447, the disclosures of which are incorporated herein by reference in their entirety. The “NO adducts” can be mono-nitrosylated, poly-nitrosylated, mono-nitrosated and/or poly-nitrosated at a variety of naturally susceptible or artificially provided binding sites for biologically active forms of nitrogen monoxide.

Suitable furoxanes include, but are not limited to, CAS1609, C93-4759, C92-4678, S35b, CHF 2206, CHF 2363, and the like.

Suitable sydnonimines include, but are not limited to, molsidomine (N-ethoxycarbonyl-3-morpholinosydnonimine), SIN-1 (3-morpholinosydnonimine) CAS 936 (3-(cis-2,6-dimethylpiperidino)-N-(4-methoxybenzoyl)-sydnonimine, pirsidomine), C87-3754 (3-(cis-2,6-dimethylpiperidino)sydnonimine, linsidomine, C4144 (3-(3,3-dimethyl-1,4-thiazane-4-yl)sydnonimine hydrochloride), C89-4095 (3-(3,3-dimethyl-1,1-dioxo-1,4-thiazane-4-yl)sydnonimine hydrochloride, and the like.

Suitable oximes, include, but are not limited to, NOR-1, NOR-3, NOR-4, and the like.

One group of NO adducts is the S-nitrosothiols, which are compounds that include at least one —S—NO group. These compounds include S-nitroso-polypeptides (the term “polypeptide” includes proteins and polyamino acids that do not possess an ascertained biological function, and derivatives thereof); S-nitrosylated amino acids (including natural and synthetic amino acids and their stereoisomers and racemic mixtures and derivatives thereof); S-nitrosylated sugars; S-nitrosylated, modified and unmodified, oligonucleotides (preferably of at least 5, and more preferably 5-200 nucleotides); straight or branched, saturated or unsaturated, aliphatic or aromatic, substituted or unsubstituted S-nitrosylated hydrocarbons; and S-nitroso heterocyclic compounds. S-nitrosothiols and methods for preparing them are described in U.S. Pat. Nos. 5,380,758 and 5,703,073; WO 97/27749; WO 98/19672; and Oae et al, Org. Prep. Proc. Int., 15(3):165-198 (1983), the disclosures of each of which are incorporated by reference herein in their entirety.

Another embodiment of the invention is S-nitroso amino acids where the nitroso group is linked to a sulfur group of a sulfur-containing amino acid or derivative thereof. Such compounds include, for example, S-nitroso-N-acetylcysteine, S-nitroso-captopril, S-nitroso-N-acetylpenicillamine, S-nitroso-homocysteine, S-nitroso-cysteine, S-nitroso-glutathione, S-nitroso-cysteinyl-glycine, and the like.

Suitable S-nitrosylated proteins include thiol-containing proteins (where the NO group is attached to one or more sulfur groups on an amino acid or amino acid derivative thereof) from various functional classes including enzymes, such as tissue-type plasminogen activator (TPA) and cathepsin B; transport proteins, such as lipoproteins; heme proteins, such as hemoglobin and serum albumin; and biologically protective proteins, such as immunoglobulins, antibodies and cytokines. Such nitrosylated proteins are described in WO 93/09806, the disclosure of which is incorporated by reference herein in its entirety. Examples include polynitrosylated albumin where one or more thiol or other nucleophilic centers in the protein are modified.

Other examples of suitable S-nitrosothiols include:

(i) HS(C(Rₑ)(R_{f}))ₘSNO;

(ii) ONS(C(Rₑ)(R_{f}))ₘRₑ; or

(iii) H₂N—CH(CO₂H)—(CH₂)ₘ—C(O)NH—CH(CH₂SNO)—C(O)NH—CH₂—CO₂H;

wherein m is an integer from 2 to 20;

Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano, an aminoalkyl, an aminoaryl, an aryl, an arylalkyl, an alkylaryl, a carboxamido, an alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, —U₃—V₅, V₆, —(C(Rₒ)(Rₚ))ₖ₁—U₃—V₅, —(C(Rₒ)(Rₚ))ₖ₁—U₃—V₆, —(C(Rₒ)(Rₚ))ₖ₁—U₃—C(O)—V₆, or Rₑ and R_{f} taken together with the carbons to which they are attached form a carbonyl, a methanthial, a heterocyclic ring, a cycloalkyl group, an aryl group, an oxime, a hydrazone, a bridged cycloalkyl group,

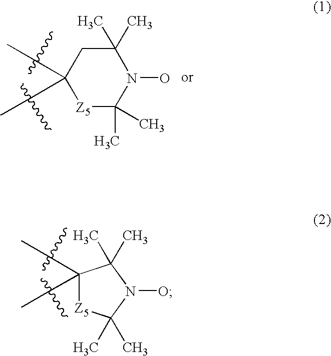

Rₒ and Rₚ are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano, an aminoalkyl, an aminoaryl, an aryl, an arylalkyl, an alkylaryl, a carboxamido, an alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, —U₃—V₅, V₆, or Rₒ and Rₚ taken together with the carbons to which they are attached form a carbonyl, a methanthial, a heterocyclic ring, a cycloalkyl group, an aryl group, an oxime, an imine, a hydrazone, a bridged cycloalkyl group,

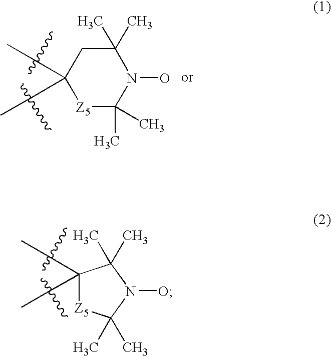

k₁ is an integer form 1 to 3;

Z₅ is —CH₂ or oxygen;

U₃ is an oxygen, sulfur- or —N(Rₐ)Rᵢ;

V₅ is —NO or —NO₂ (i.e. an oxidized nitrogen);

Rₐ is a lone pair of electrons, a hydrogen or an alkyl group;

Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, arylsulphonyloxy, a sulfonamido, a carboxamido, a carboxylic ester, an aminoalkyl, an aminoaryl, —CH₂—C(U₃—V₅)(Rₑ)(R_{f}), a bond to an adjacent atom creating a double bond to that atom or —(N₂O₂—)⁻.M₁⁺, wherein M₁⁺ is an organic or inorganic cation.

In cases where Rₑ and R_{f} are independently a heterocyclic ring or taken together Rₑ and R_{f} are a heterocyclic ring, then Rᵢ can be a substituent on any disubstituted nitrogen contained within the radical wherein Rᵢ is as defined herein.

Nitrosothiols can be prepared by various methods of synthesis. In general, the thiol precursor is prepared first, then converted to the S-nitrosothiol derivative by nitrosation of the thiol group with NaNO₂ under acidic conditions (pH is about 2.5) which yields the S-nitroso derivative. Acids which can be used for this purpose include aqueous sulfuric, acetic and hydrochloric acids. The thiol precursor can also be nitrosylated by reaction with an organic nitrite such as tert-butyl nitrite, or a nitrosonium salt such as nitrosonium tetrafluoroborate in an inert solvent.

Another group of NO adducts for use in the invention, where the NO adduct is a compound that donates, transfers or releases nitric oxide, include compounds comprising at least one ON—O— or ON—N— group. The compounds that include at least one ON—O— or ON—N-group are preferably ON—O— or ON—N-polypeptides (the term “polypeptide” includes proteins and polyamino acids that do not possess an ascertained biological function, and derivatives thereof); ON—O— or ON—N-amino acids (including natural and synthetic amino acids and their stereoisomers and racemic mixtures); ON—O— or ON—N-sugars; ON—O— or —ON—N— modified or unmodified oligonucleotides (comprising at least 5 nucleotides, preferably 5-200 nucleotides); ON—O— or ON—N— straight or branched, saturated or unsaturated, aliphatic or aromatic, substituted or unsubstituted hydrocarbons; and ON—O—, ON—N— or ON—C-heterocyclic compounds. Examples of compounds comprising at least one ON—O— or ON—N-group include butyl nitrite, isobutyl nitrite, tert-butyl nitrite, amyl nitrite, isoamyl nitrite, N-nitrosamines, N-nitrosamides, N-nitrosourea, N-nitrosoguanidines, N-nitrosocarbamates, N-acyl-N-nitroso compounds (such as, N-methyl-N-nitrosourea); N-hydroxy-N-nitrosamines, cupferron, alanosine, dopastin, 1,3-disubstitued nitrosiminobenzimidazoles, 1,3,4-thiadiazole-2-nitrosimines, benzothiazole-2(3H)-nitrosimines, thiazole-2-nitrosimines, oligonitroso sydnonimines, 3-alkyl-N-nitroso-sydnonimines, 2H-1,3,4-thiadiazine nitrosimines.

Another group of NO adducts for use in the invention include nitrates that donate, transfer or release nitric oxide, such as compounds comprising at least one O₂N—O—, O₂N—N— or O₂N—S— group. Among these compounds are O₂N—O—, O₂N—N— or O₂N—S— polypeptides (the term “polypeptide” includes proteins and also polyamino acids that do not possess an ascertained biological function, and derivatives thereof); O₂N—O—, O₂N—N— or O₂N—S— amino acids (including natural and synthetic amino acids and their stereoisomers and racemic mixtures); O₂N—O—, O₂N—N— or O₂N—S— sugars; O₂N—O—, O₂N—N— or O₂N—S— modified and unmodified oligonucleotides (comprising at least 5 nucleotides, preferably 5-200 nucleotides); O₂N—O—, O₂N—N— or O₂N—S— straight or branched, saturated or unsaturated, aliphatic or aromatic, substituted or unsubstituted hydrocarbons; and O₂N—O—, O₂N—N— or O₂N—S— heterocyclic compounds. Examples of compounds comprising at least one O₂N—O—, O₂N—N— or O₂N—S-group include isosorbide dinitrate, isosorbide mononitrate, clonitrate, erythrityl tetranitrate, mannitol hexanitrate, nitroglycerin, pentaerythritoltetranitrate, pentrinitrol, propatylnitrate and organic nitrates with a sulfhydryl-containing amino acid such as, for example SPM 3672, SPM 4757, SPM 5185, SPM 5186 and those disclosed in U.S. Pat. Nos. 5,284,872, 5,428,061, 5,661,129, 5,807,847 and 5,883,122 and in WO 97/46521, WO 00/54756 and in WO 03/013432, the disclosures of each of which are incorporated by reference herein in their entirety.

Another group of NO adducts are N-oxo-N-nitrosoamines that donate, transfer or release nitric oxide and are represented by the formula: R^{1″}R^{2″}N—N(O-M⁺)—NO, where R^{1″} and R^{2″} are each independently a polypeptide, an amino acid, a sugar, a modified or unmodified oligonucleotide, a straight or branched, saturated or unsaturated, aliphatic or aromatic, substituted or unsubstituted hydrocarbon, or a heterocyclic group, and where M₁⁺ is an organic or inorganic cation, such, as for example, an alkyl substituted ammonium cation or a Group I metal cation.

The invention is also directed to compounds that stimulate endogenous NO or elevate levels of endogenous endothelium-derived relaxing factor (EDRF) in vivo or are oxidized to produce nitric oxide and/or are substrates for nitric oxide synthase and/or cytochrome P450. Such compounds include, for example, L-arginine, L-homoarginine, and N-hydroxy-L-arginine, N-hydroxy-L-homoarginine, N-hydroxydebrisoquine, N-hydroxypentamidine including their nitrosated and/or nitrosylated analogs (e.g., nitrosated L-arginine, nitrosylated L-arginine, nitrosated N-hydroxy-L-arginine, nitrosylated N-hydroxy-L-arginine, nitrosated and nitrosylated L-homoarginine), N-hydroxyguanidine compounds, amidoxime, ketoximes, aldoxime compounds, that can be oxidized in vivo to produce nitric oxide. Compounds that may be substrates for a cytochrome P450, include, for example, imino(benzylamino)methylhydroxylamine, imino(((4-methylphenyl)methyl)amino)methylhydroxylamine, imino(((4-methoxyphenyl)methyl)amino) methylhydroxylamine, imino(((4-(trifluoromethyl)phenyl)methyl)amino) methylhydroxylamine, imino(((4-nitrophenyl)methyl)amino)methylhydroxylamine, (butylamino) iminomethylhydroxylamine, imino (propylamino) methylhydroxylamine, imino(pentylamino)methylhydroxylamine, imino (propylamino)methylhydroxylamine, imino ((methylethyl)amino)methylhydroxylamine, (cyclopropylamino) iminomethylhydroxylamine, imino-2-1,2,3,4-tetrahydroisoquinolyl methylhydroxylamine, imino(1-methyl(2-1,2,3,4-tetrahydroisoquinolyl))methylhydroxylamine, (1,3-dimethyl(2-1,2,3,4-tetrahydroisoquinolyl)) iminomethylhydroxylamine, (((4-chlorophenyl)methyl)amino)iminomethylhydroxylamine, ((4-chlorophenyl)amino) iminomethylhydroxylamine, (4-chlorophenyl)(hydroxyimino) methylamine, and 1-(4-chlorophenyl)-1-(hydroxyimino) ethane, and the like, precursors of L-arginine and/or physiologically acceptable salts thereof, including, for example, citrulline, ornithine, glutamine, lysine, polypeptides comprising at least one of these amino acids, inhibitors of the enzyme arginase (e.g., N-hydroxy-L-arginine and 2(S)-amino-6-boronohexanoic acid), nitric oxide mediators and/or physiologically acceptable salts thereof, including, for example, pyruvate, pyruvate precursors, α-keto acids having four or more carbon atoms, precursors of α-keto acids having four or more carbon atoms (as disclosed in WO 03/017996, the disclosure of which is incorporated herein in its entirety), and the substrates for nitric oxide synthase, cytokines, adenosin, bradykinin, calreticulin, bisacodyl, and phenolphthalein. EDRF is a vascular relaxing factor secreted by the endothelium, and has been identified as nitric oxide (NO) or a closely related derivative thereof (Palmer et al, Nature, 327:524-526 (1987); Ignarro et al, Proc. Natl. Acad. Sci. USA, 84:9265-9269 (1987)).

The invention is also directed to nitric oxide enhancing compounds that can increase endogenous nitric oxide. Such compounds, include for example, nitroxide containing compounds, include, but are not limited to, substituted 2,2,6,6-tetramethyl-1-piperidinyloxy compounds, substituted 2,2,5,5-tetramethyl-3-pyrroline-1-oxyl compounds, substituted 2,2,5,5-tetramethyl-1-pyrrolidinyloxyl compounds, substituted 1,1,3,3-tetramethylisoindolin-2-yloxyl compounds, substituted 2,2,4,4-tetramethyl-1-oxazolidinyl-3-oxyl compounds, substituted 3-imidazolin-1-yloxy, 2,2,5,5-tetramethyl-3-imidazolin-1-yloxyl compounds, OT-551,4-hydroxy-2,2,6,6-tetramethyl-1-piperidinyloxy (tempol), and the like. Suitable substituents, include, but are not limited to, aminomethyl, benzoyl, 2-bromoacetamido, 2-(2-(2-bromoacetamido)ethoxy)ethylcarbamoyl, carbamoyl, carboxy, cyano, 5-(dimethylamino)-1-naphthalenesulfonamido, ethoxyfluorophosphinyloxy, ethyl, 5-fluoro-2,4-dinitroanilino, hydroxy, 2-iodoacetamido, isothiocyanato, isothiocyanatomethyl, methyl, maleimido, maleimidoethyl, 2-(2-maleimidoethoxy)ethylcarbamoyl, maleimidomethyl, maleimido, oxo, phosphonooxy, and the like.

The invention is also based on the discovery that compounds and compositions of the invention may be used in conjunction with other therapeutic agents for co-therapies, partially or completely, in place of other conventional antiinflammatory compounds, such as, for example, together with steroids, COX-2 inhibitors, NSAIDs, 5-lipoxygenase (5-LO) inhibitors, leukotriene B₄ (LTB₄) receptor antagonists, leukotriene A₄ (LTA₄) hydrolase inhibitors, 5-HT agonists, anti-hyperlipidemic compounds, H₂ receptor antagonists, antineoplastic agents, antiplatelet agents, thrombin inhibitors, thromboxane inhibitors, carbonic anhydrase inhibitors, decongestants, diuretics, sedating or non-sedating anti-histamines, inducible nitric oxide synthase inhibitors, opiods, analgesics, Helicobacter pylori inhibitors, proton pump inhibitors, isoprostane inhibitors, compounds used for the treatment of glaucoma, and combinations of two or more thereof. The therapeutic agent may optionally be nitrosated and/or nitrosylated and/or contain at least one heterocyclic nitric oxide donor group.

Leukotriene A₄ (LTA₄) hydrolase inhibitors refer to compounds that selectively inhibit leukotriene A₄ hydrolase with an IC₅₀ of less than about 10 μM, and preferably with an IC₅₀ of less than about 1 μM. Suitable LTA₄ hydrolase inhibitors include, but are not limited to, RP-64966, (S,S)-3-amino-4-(4-benzyloxyphenyl)-2-hydroxybutyric acid benzyl ester, N-(2(R)-(cyclohexylmethyl)-3-(hydroxycarbamoyl)propionyl)-L-alanine, 7-(4-(4-ureidobenzyl)phenyl) heptanoic acid and 3 (3-(1E,3E-tetradecadienyl)-2-oxiranyl)benzoic acid lithium salt, and mixtures of two or more thereof.

Suitable LTB₄ receptor antagonists include, but are not limited to, ebselen, linazolast, ontazolast; WAY 121006; Bay-x-1005; BI-RM-270; CGS-25019C; ETH-615; MAFP; TMK-688; T-0757; LY 213024, LY 210073, LY 223982, LY 233469, LY 255283, LY 264086, LY 292728 and LY 293111; ONO-LB457, ONO-4057, and ONO-LB-448, S-2474, calcitrol; PF 10042; Pfizer 105696; RP 66153; SC-53228, SC-41930, SC-50605, SC-51146 and SC-53228; SB-201146 and SB-209247; SKF-104493; SM 15178; TMK-688; BPC 15, and mixtures of two or more thereof. The preferred LTB₄ receptor antagonists are calcitrol, ebselen, Bay-x-1005, CGS-25019C, ETH-615, LY-293111, ONO-4057 and TMK-688, and mixtures of two or more thereof.

Suitable 5-LO inhibitors include, but are not limited to, A-76745, 78773 and ABT761; Bay-x-1005; CMI-392; E-3040; EF-40; F-1322; ML-3000; PF-5901; R-840; rilopirox, flobufen, linasolast, lonapolene, masoprocol, ontasolast, tenidap, zileuton, pranlukast, tepoxalin, rilopirox, flezelastine hydrochloride, enazadrem phosphate, and bunaprolast, and mixtures of two or more thereof. Suitable 5-LO inhibitors are also described more fully in WO 97/29776, the disclosure of which is incorporated herein by reference in its entirety.

Suitable 5-HT agonists, include, but are not limited to, rizatriptan, sumatriptan, naratriptan, zolmitroptan, eleptriptan, almotriptan, ergot alkaloids. ALX 1323, Merck L 741604 SB 220453 and LAS 31416. Suitable 5-HT agonists are described more fully in WO 0025779, and in WO 00/48583. 5-HT agonists refers to a compound that is an agonist to any 5-HT receptor, including but not limited to, 5-HT₁ agonists, 5-HT_{1B} agonists and 5-HT_{1D} agonists, and the like.

Suitable steroids, include, but are not limited to, budesonide, dexamethasone, corticosterone, prednisolone, and the like. Suitable steroids are described more fully in the literature, such as in the Merck Index on CD-ROM, 13^{th} Edition.

Suitable anti-hyperlipidemic compounds include, but are not limited to, statins or HMG-CoA reductase inhibitors, such as, for example, atorvastatin (LIPITOR®), bervastatin, cerivastatin (BAYCOL®), dalvastatin, fluindostatin (Sandoz XU-62-320), fluvastatin, glenvastatin, lovastatin (MEVACOR®), mevastatin, pravastatin (PRAVACHOL®), rosuvastatin (CRESTRO®), simvastatin (ZOCOR®), velostatin (also known as synvinolin), VYTORIN™ (ezetimibe/simvastatin), GR-95030, SQ 33,600, BMY 22089, BMY 22,566, CI-980, and the like; gemfibrozil, cholystyramine, colestipol, niacin, nicotinic acid, bile acid sequestrants, such as, for example, cholestyramine, colesevelam, colestipol, poly(methyl-(3-trimethylaminopropyl) imino-trimethylene dihalide) and the like; probucol; fibric acid agents or fibrates, such as, for example, bezafibrate (Bezalip™), beclobrate, binifibrate, ciprofibrate, clinofibrate, clofibrate, etofibrate, fenofibrate (Lipidil™, Lipidil Micro™), gemfibrozil (Lopid™), nicofibrate, pirifibrate, ronifibrate, simfibrate, theofibrate and the like; cholesterol ester transfer protein (CETP) inhibitors, such as for example, CGS 25159, CP-529414 (torcetrapid), JTT-705, substituted N-[3-(1,1,2,2-tetrafluoroethoxy)benzyl]-N-(3-phenoxyphenyl)-trifluoro-3-amino-2-propanols, N,N-disubstituted trifluoro-3-amino-2-propanols, PD 140195 (4-phenyl-5-tridecyl-4H-1,2,4-triazole-3-thiol), SC-794, SC-795, SCH 58149, and the like.

In some embodiments the anti-hyperlipidemic compounds are atorvastatin, fluvastatin, lovastatin, pravastatin, rosuvastatin or simvastatin. In more particular embodiments the atorvastatin is administered in an amount of about 10 milligrams to about 80 milligrams as a single dose or as multiple doses per day; the fluvastatin is administered in an amount of about 20 milligrams to about 80 milligrams as a single does or as multiple doses per day; the lovastatin is administered in an amount of about 10 milligrams to about 80 milligrams as a single dose or as multiple doses per day; the pravastatin is administered in an amount of about 10 milligrams to about 80 milligrams as a single dose or as multiple doses per day; the rosuvastatin is administered in an amount of about 5 milligrams to about 40 milligrams as a single dose or as multiple doses per day; the simvastatin is administered in an amount of about 5 milligrams to about 80 milligrams as a single dose or as multiple doses per day.

Suitable COX-2 inhibitors include, but are not limited to, nimesulide, celecoxib (CELEBREX®), etoricoxib (ARCOXIA®), flosulide, lumiracoxib (PREXIG®, COX-189), parecoxib (DYNSTAT®), rofecoxib (VIOXX®), tiracoxib (JTE-522), valdecoxib (BEXTRA®), ABT 963, BMS 347070, CS 502, DuP 697, GW-406381, NS-386, SC-57666, SC-58125, SC-58635, and the like, and mixtures of two or more thereof. Suitable COX-2 inhibitors are in U.S. Pat. Nos. 5,344,991, 5,380,738, 5,393,790, 5,409,944, 5,434,178, 5,436,265, 5,466,823, 5,474,995, 5,510,368, 5,536,752, 5,550,142, 5,552,422, 5,604,253, 5,604,260, 5,639,780, 5,932,598 and 6,633,272, and in WO 94/03387, WO 94/15723, WO 94/20480, WO 94/26731, WO 94/27980, WO 95/00501, WO 95/15316, WO 96/03387, WO 96/03388, WO 96/06840, WO 96/21667, WO 96/31509, WO 96/36623, WO 97/14691, WO 97/16435, WO 01/45703 and WO 01/87343, the disclosures of each of which are incorporated herein by reference in their entirety; and in the literature, such as in Goodman and Gilman, The Pharmacological Basis of Therapeutics (9th Edition), McGraw-Hill, 1995; and the Merck Index on CD-ROM, Thirteenth Edition; and on STN Express, file phar and file registry.

In some embodiments the COX-2 inhibitors are celecoxib, etoracoxib, lumiracoxib, paracoxib, rofecoxib or valdecoxib. In more particular embodiments the celecoxib is administered in an amount of about 100 milligrams to about 800 milligrams as a single dose or as multiple doses per day; the etoricoxib is administered in an amount of about 50 milligrams to about 200 milligrams as a single does or as multiple doses per day; the lumiracoxib is administered in an amount of about 40 milligrams to about 1200 milligrams as a single does or as multiple doses per day; the paracoxib is administered in an amount of about 20 milligrams to about 100 milligrams as a single does or as multiple doses per day; the rofecoxib is administered in an amount of about 12.5 milligrams to about 50 milligrams as a single does or as multiple doses per day; the valdecoxib is administered in an amount of about 10 milligrams to about 40 milligrams as a single does or as multiple doses per day.

Suitable NSAIDs include, but are not limited to, acetaminophen, acemetacin, aceclofenac, alminoprofen, amfenac, bendazac, benoxaprofen, bromfenac, bucloxic acid, butibufen, carprofen, cinmetacin, clopirac, diclofenac, etodolac, felbinac, fenclozic acid, fenbufen, fenoprofen, fentiazac, flunoxaprofen, flurbiprofen, ibufenac, ibuprofen, indomethacin, isofezolac, isoxepac, indoprofen, ketoprofen, lonazolac, loxoprofen, metiazinic acid, mofezolac, miroprofen, naproxen, oxaprozin, pirozolac, pirprofen, pranoprofen, protizinic acid, salicylamide, sulindac, suprofen, suxibuzone, tiaprofenic acid, tolmetin, xenbucin, ximoprofen, zaltoprofen, zomepirac, aspirin, acemetcin, bumadizon, carprofenac, clidanac, diflunisal, enfenamic acid, fendosal, flufenamic acid, flunixin, gentisic acid, ketorolac, meclofenamic acid, mefenamic acid, mesalamine, prodrugs thereof, and the like. Suitable NSAIDs are described more fully in the literature, such as in Goodman and Gilman, The Pharmacological Basis of Therapeutics (9th Edition), McGraw-Hill, 1995, Pgs. 617-657; the Merck Index on CD-ROM, 13^{th} Edition; and in U.S. Pat. Nos. 6,057,347 and 6,297,260 assigned to NitroMed. Inc., the disclosures of which are incorporated herein by reference in their entirety.

In some embodiments the NSAIDs are acetaminophen, diclofenac, flurbiprofen, ibuprofen, indomethacin, ketoprofen, naproxen or aspirin. In more particular embodiments the acetaminophen is administered in an amount of about 325 milligrams to about 4 grams as a single dose or as multiple doses per day; the diclofenac is administered in an amount of about 50 milligrams to about 250 milligrams as a single does or as multiple doses per day; the flurbiprofen is administered in an amount of about 100 milligrams to about 300 milligrams as a single does or as multiple doses per day; the ibuprofen is administered in an amount of about 400 milligrams to about 3.2 grams as a single does or as multiple doses per day; the indomethacin is administered in an amount of about 25 milligrams to about 200 milligrams as a single does or as multiple doses per day; the ketoprofen is administered in an amount of about 50 milligrams to about 300 milligrams as a single does or as multiple doses per day; the naproxen is administered in an amount of about 250 milligrams to about 1.5 grams as a single does or as multiple doses per day; the aspirin is administered in an amount of about 10 milligrams to about 2 grams as a single does or as multiple doses per day.

Suitable H₂ receptor anatgonists, include, but are not limited to, cimetidine, roxatidine, rantidine and the like. Suitable H₂ receptor antagonists are described more fully in the literature, such as in Goodman and Gilman, The Pharmacological Basis of Therapeutics (9th Edition), McGraw-Hill, 1995, Pgs. 901-915; the Merck Index on CD-ROM, 13^{th} Edition; and in WO 00/28988 assigned to NitroMed. Inc., the disclosures of which are incorporated herein by reference in their entirety.

Suitable antineoplastic agents, include but are not limited to, 5-FU-fibrinogen, acanthifolic acid, aminothiadiazole, altretamine, anaxirone, aclarubicin and the like. Suitable antineoplastic agents are also described in U.S. Pat. No. 6,025,353 and WO 00/38730, the disclosures of which are incorporated herein by reference in their entirety.

Suitable antiplatelet agents, include but are not limited to, aspirin, ticlopidine, dipyridamole, clopidogrel, glycoprotein IIb/IIIa receptor antagonists, and the like. Suitable antineoplastic agents are also described in WO 99/45913, the disclosure of which is incorporated herein by reference in its entirety. In a preferred embodiment of the invention, the antiplatelet agent is aspirin, more preferably, low-dose aspirin (i.e. 75 mg-100 mg/day).

Suitable thrombin inhibitors, include but are not limited to, N′-((1-(aminoiminomethyl)-4-piperidinyl)methyl)-N-(3,3-diphenylpropinyl)-L-proline amide), 3-(2-phenylethylamino)-6-methyl-1-(2-amino-6-methyl-5-methylene-carboxamidomethylpyridinyl)-2-pyrazinone, 3-(2-phenethylamino)-6-methyl-1-(2-amino-6-methyl-5-methylenecarboxamidomethylpyridinyl)-2-pyridinone, and the like. Suitable thrombin inhibitors are also described in WO 00/18352, the disclosure of which is incorporated herein by reference in its entirety.

Suitable thromboxane inhibitors, include but are not limited to thromboxane synthase inhibitors, thromboxane receptor antagonists, and the like. Suitable thromboxane inhibitors, are also described in WO 01/87343, the disclosure of which is incorporated herein by reference in its entirety.

Suitable carbonic anhydrase inhibitors, include, but are not limited to, acetazolamide, brinzolamide, dorzolamide, ethoxzolamide, 6-hydroxy-2-benzothiazolesulfonamide, methazolamide, thiophene sulfonamide, an aromatic sulfonamide, an ester of 6-hydroxy-2-benzothiazolesulfonamide, an ester of 5-hydroxy-2-benzothiazolesulfonamide, and the like. Suitable carbonic anhydrase inhibitors are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

In some embodiments the carbonic anhydrase inhibitors are brinzolamide and dorzolamide.

Suitable decongestants include, but are not limited to, phenylephrine, phenylpropanolamine, pseudophedrine, oxymetazoline, ephinephrine, naphazoline, xylometazoline, propylhexedrine, levo-desoxyephedrine, and the like.

Suitable diuretics include but are not limited to, thiazides (such as, for example, althiazide, bendroflumethiazide, benzclortriazide, benzhydrochlorothiazide, benzthiazide, buthiazide, chlorothiazide, cyclopenethiazide, cyclothiazide, epithiazide, ethiazide, hydrobenzthiazide, hydrochlorothiazide, hydroflumethiazide, methylclothiazide, methylcyclothiazide, penflutazide, polythiazide, teclothiazide, trichlormethiazide, triflumethazide, and the like); alilusem, ambuside, amiloride, aminometradine, azosemide, bemetizide, bumetanide, butazolamide, butizide, canrenone, carperitide, chloraminophenamide, chlorazanil, chlormerodrin, chlorthalidone, cicletanide, clofenamide, clopamide, clorexolone, conivaptan, daglutril, dichlorophenamide, disulfamide, ethacrynic acid, ethoxzolamide, etozolon, fenoldopam, fenquizone, furosemide, indapamide, mebutizide, mefruside, meralluride, mercaptomerin sodium, mercumallylic acid, mersalyl, methazolamide, meticane, metolazone, mozavaptan, muzolimine, N-(5-1,3,4-thiadiazol-2-yl)acetamide, nesiritide, pamabrom, paraflutizide, piretanide, protheobromine, quinethazone, scoparius, spironolactone, theobromine, ticrynafen, torsemide, torvaptan, triamterene, tripamide, ularitide, xipamide or potassium, AT 189000, AY 31906, BG 9928, BG 9791, C 2921, DTI 0017, JDL 961, KW 3902, MCC 134, SLV 306, SR 121463, WAY 140288, ZP 120, and the like. Suitable diuretics are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

Depending on the diuretic employed, potassium may also be administered to the patient in order to optimize the fluid balance while avoiding hypokalemic alkalosis. The administration of potassium can be in the form of potassium chloride or by the daily ingestion of foods with high potassium content such as, for example, bananas or orange juice. The method of administration of these compounds is described in further detail in U.S. Pat. No. 4,868,179, the disclosure of which is incorporated by reference herein in its entirety.

In some embodiments the diuretics are amiloride, furosemide, chlorthalidone, hydrochlorothiazide or triamterene. In more particular embodiments the amiloride is administered as amiloride hydrochloride in an amount of about 5 milligrams to about 15 milligrams as a single dose or as multiple doses per day; the furosemide is administered in an amount of about 10 milligrams to about 600 milligrams as a single dose or as multiple doses per day; the chlorthalidone is administered in an amount of about 15 milligrams to about 150 milligrams as a single dose or as multiple doses per day; the hydrochlorothiazide is administered in an amount of about 12.5 milligrams to about 300 milligrams as a single dose or as multiple doses per day; the triamterene is administered in an amount of about 35 milligrams to about 225 milligrams as a single dose or as multiple doses per day.

Suitable antitussives include, but are not limited to, codeine, hydrocodone, caramiphen, carbetapentane, dextramethorphan, and the like.

Suitable phosphodiesterase inhibitors, include but are not limited to, filaminast, piclamilast, rolipram, Org 20241, MC1-154, roflumilast, toborinone, posicar, lixazinone, zaprinast, sildenafil, pyrazolopyrimidinones, motapizone, pimobendan, zardaverine, siguazodan, CI-930, EMD 53998, imazodan, saterinone, loprinone hydrochloride, 3-pyridinecarbonitrile derivatives, acefylline, albifylline, bamifylline, denbufyllene, diphylline, doxofylline, etofylline, torbafylline, theophylline, nanterinone, pentoxofylline, proxyphylline, cilostazol, cilostamide, MS 857, piroximone, milrinone, aminone, tolafentrine, dipyridamole, papaveroline, E4021, thienopyrimidine derivatives, triflusal, ICOS-351, tetrahydropiperazino(1,2-b)beta-carboline-1,4-dione derivatives, carboline derivatives, 2-pyrazolin-5-one derivatives, fused pyridazine derivatives, quinazoline derivatives, anthranilic acid derivatives, imidazoquinazoline derivatives, tadalafil, vardenafil, and in Goodman and Gilman, The Pharmacological Basis of Therapeutics (9th Ed.), McGraw-Hill, Inc. (1995), The Physician's Desk Reference (49th Ed.), Medical Economics (1995), Drug Facts and Comparisons (1993 Ed), Facts and Comparisons (1993), and the Merck Index on CD-ROM, 13^{th} Edition; and the like. Phosphodiesterase inhibitors and their nitrosated and/or nitrosylated derivatives are also disclosed in U.S. Pat. Nos. 5,932,538, 5,994,294, 5,874,437, 5,958,926 reissued as U.S. Pat. No. RE 03772346,172,060, 6,197,778, 6,177,428, 6,172,068, 6,221,881, 6,232,321, 6,197,782, 6,133,272, 6,211,179, 6,316,457 and 6,331,542, the disclosures of each of which are incorporated herein by reference in their entirety.

Suitable proton pump inhibitors include, but are not limited to, disulprazole, esomeprazole, lansoprazole, leminoprazole, omeprazole, pantoprazole, rabeprazole, timoprazole, tenatoprazole, 2-(2-benzimidazolyl)-pyridine, tricyclic imidazole, thienopydidine benzimidazole, fluoroalkoxy substituted benzimidazole, dialkoxy benzimidazole, N-substituted 2-(pyridylalkenesulfinyl)benzimidazole, cycloheptenepyridine, 5-pyrrolyl-2-pyridylmethylsulfinyl benzimidazole, alkylsulfinyl benzimidazole, fluoro-pyridylmethylsulfinyl benzimidazole, imidazo[4,5-b]pydridine, RO 18-5362, IY 81149,4-amino-3-carbonyl quinoline, 4-amino-3-acylnaphthyride, 4-aminoquinoline, 4-amino-3-acylquinoline, 3-butyryl-4-(2-methylphenylamino)-8-(2-hydroxyethoxy)quinoline, quinazoline, tetrahydroisoquinolin-2-yl pyrimidine, YH 1885, 3-substituted 1,2,4-thiadiazolo(4,5-a) benzimidazole, 3-substituted imidazo(1,2-d)-thiadiazole, 2-sulfinylnicotinamide, pyridylsulfinylbenz imidazole, pyridylsulfinyl thieno imidazole, theinoimidazole-toluidine, 4,5-dihydrooxazole, thienoimidazole-toluidine, Hoe-731, imidazo[1,2-a]pyridine, pyrrolo[2,3-b]pyridine, and the like. Suitable proton pump inhibitors are described more fully in the literature, such as in Goodman and Gilman, The Pharmacological Basis of Therapeutics (9th Edition), McGraw-Hill, 1995; the Merck Index on CD-ROM, 13^{th} Edition; and in WO 00/50037 assigned to NitroMed. Inc., the disclosures of which are incorporated herein by reference in their entirety.

Suitable compounds used for the treatment of glaucoma, include, but are not limited to, acetylcholinesterase inhibitors (such as, for example, citicoline, donepezil, heptatigmine, galantamine, metafonate, physostignine, rivastignine, tarcine, velnacrine, and the like) carbachol, pilocarpine and the like. Suitable compounds used for the treatment of glaucoma are described more fully in the literature, such as in Goodman and Gilman, The Pharmacological Basis of Therapeutics (9th Edition), McGraw-Hill, 1995; and the Merck Index on CD-ROM, 13^{th} Edition; and on STN Express, file phar and file registry.

The compounds and compositions of the invention, may also be used in combination therapies with opioids and other analgesics, including, but not limited to, narcotic analgesics, Mu receptor antagonists, Kappa receptor antagonists, non-narcotic (i.e. non-addictive) analgesics, monoamine uptake inhibitors, adenosine regulating agents, cannabinoid derivatives, neurokinin 1 receptor antagonists, Substance P antagonists, neurokinin-1 receptor antagonists, sodium channel blockers, N-methyl-D-aspartate receptor antagonists, and mixtures of two or more thereof. Preferred combination therapies would be with morphine, meperidine, codeine, pentazocine, buprenorphine, butorphanol, dezocine, meptazinol, hydrocodone, oxycodone, methadone, Tramadol ((+) enantiomer), DuP 747, Dynorphine A, Enadoline, RP-60180, HN-11608, E-2078, ICI-204448, acetominophen (paracetamol), propoxyphene, nalbuphine, E-4018, filenadol, mirtentanil, amitriptyline, DuP631, Tramadol ((−) enantiomer), GP-531, acadesine, AKI-1, AKI-2, GP-1683, GP-3269, 4030W92, tramadol racemate, Dynorphine A, E-2078, AXC3742, SNX-111, ADL2-1294, ICI-204448, CT-3, CP-99,994, CP-99,994, and mixtures of two or more thereof.

The compounds and compositions of the invention can also be used in combination with inducible nitric oxide synthase (iNOS) inhibitors. Suitable iNOS inhibitors are disclosed in U.S. Pat. Nos. 5,132,453 and 5,273,875, and in WO 97/38977 and WO 99/18960, the disclosures of each of which are incorporated by reference herein in their entirety.

The invention provides compositions comprising (i) an organic nitric oxide enhancing salt of a NSAID, (ii) a nitric oxide enhancing compound, such as, isosorbide dinitrate and/or isosorbide mononitrate (such as, isosorbide dinitrate), and (i) a hydralazine compound (such as, hydralazine hydrochloride). In one embodiment, the hydralazine hydrochloride can be administered in an amount of about 30 milligrams per day to about 400 milligrams per day; the isosorbide dinitrate can be administered in an amount of about 10 milligrams per day to about 200 milligrams per day; or the isosorbide mononitrate can be administered in an amount of about 5 milligrams per day to about 120 milligrams per day. In another embodiment, the hydralazine hydrochloride can be administered in an amount of about 50 milligrams per day to about 300 milligrams per day; the isosorbide dinitrate can be administered in an amount of about 20 milligrams per day to about 160 milligrams per day; or the isosorbide mononitrate can be administered in an amount of about 15 milligrams per day to about 100 milligrams per day. In yet another embodiment, the hydralazine hydrochloride can be administered in an amount of about 37.5 milligrams to about 75 milligrams one to four times per day; the isosorbide dinitrate can be administered in an amount of about 20 milligrams to about 40 milligrams one to four times per day; or the isosorbide mononitrate can be administered in an amount of about 10 milligrams to about 20 milligrams one to four times per day. In another embodiment of the methods of the invention, the patient can be administered a composition comprising about 225 mg hydralazine hydrochloride and about 120 mg isosorbide dinitrate once per day (i.e., q.d.). In another embodiment of the methods of the invention, the patient can be administered a composition comprising about 112.5 mg hydralazine hydrochloride and about 60 mg isosorbide dinitrate twice per day (i.e., b.i.d.). In another embodiment of the methods of the invention, the patient can be administered a composition comprising about 56.25 mg hydralazine hydrochloride and about 30 mg isosorbide dinitrate twice per day (i.e., b.i.d.). In another embodiment of the methods of the invention, the patient can be administered a composition comprising about 75 mg hydralazine hydrochloride and about 40 mg isosorbide dinitrate three times per day (i.e., t.i.d.). In another embodiment of the methods of the invention, the patient can be administered a composition comprising about 37.5 mg hydralazine hydrochloride and about 20 mg isosorbide dinitrate three times per day (i.e., t.i.d.). The particular amounts of hydralazine and isosorbide dinitrate or isosorbide mononitrate can be administered as a single dose once a day; or in multiple doses several times throughout the day; or as a sustained-release oral formulation, or as an injectable formulation.

The invention provides methods for treating inflammation, pain (both chronic and acute), and fever, such as, for example, analgesic in the treatment of pain, including, but not limited to headaches, migraines, postoperative pain, dental pain, muscular pain, and pain resulting from cancer; as an antipyretic for the treatment of fever, including but not limited to, rheumatic fever, symptoms associated with influenza or other viral infections, common cold, low back and neck pain, dysmenorrhea, headache, toothache, sprains, strains, myositis, neuralgia, synovitis, menstrual cramps; arthritis, including but not limited to rheumatoid arthritis, degenerative joint disease (osteoarthritis), spondyloarthropathies, gouty arthritis, systemic lupus erythematosus and juvenile arthritis by administering to the patient in need thereof an effective amount of the compounds and/or compositions described herein. For example, the patient can be administered an effective amount of at least one organic nitric oxide enhancing salt of an NSAID. In another embodiment, the patient can be administered an effective amount of at least one organic nitric oxide enhancing salt of an NSAID, and at least one nitric oxide enhancing compound. In yet another embodiment, the patient can be administered an effective amount of at least one organic nitric oxide enhancing salt of an NSAID, and, at least one therapeutic agent, including but not limited to, such as, for example, steroids, cyclooxygenase-2 (COX-2) inhibitors, nonsteroidal antiinflammatory compounds (NSAID), 5-lipoxygenase (5-LO) inhibitors, leukotriene B₄ (LTB₄) receptor antagonists, leukotriene A₄ (LTA₄) hydrolase inhibitors, 5-HT agonists, anti-hyperlipidemic compounds, H₂ antagonists, antineoplastic agents, antiplatelet agents, thrombin inhibitors, thromboxane inhibitors, carbonic anhydrase inhibitors, decongestants, diuretics, sedating or non-sedating anti-histamines, inducible nitric oxide synthase inhibitors, opioids, analgesics, Helicobacter pylori inhibitors, phosphodiesterase inhibitors, proton pump inhibitors, isoprostane inhibitors, compounds used for the treatment of glaucoma, and combinations of two or more thereof. In another embodiment, the patient can be administered an effective amount of at least one organic nitric oxide enhancing salt of an NSAID, and, at least one therapeutic agent, and, at least one nitric oxide enhancing compound. The organic nitric oxide donor salts of NSAIDs, nitric oxide enhancing compounds, and/or therapeutic agents can be administered separately or as components of the same composition in one or more pharmaceutically acceptable carriers.

The invention provides methods for treating gastrointestinal disorders by administering to the patient in need thereof an effective amount of the compounds and/or compositions described herein. Such gastrointestinal disorders refer to any disease or disorder of the upper gastrointestinal tract (e.g., esophagus, the stomach, the duodenum, jejunum) including, for example, inflammatory bowel disease, Crohn's disease, gastritis, irritable bowel syndrome, constipation, ulcerative colitis, peptic ulcers, stress ulcers, gastric hyperacidity, dyspepsia, gastroparesis, Zollinger-Ellison syndrome, gastroesophageal reflux disease, bacterial infections (including, for example, a Helicobacter Pylori associated disease), short-bowel (anastomosis) syndrome, hypersecretory states associated with systemic mastocytosis or basophilic leukemia and hyperhistaminemia, and bleeding peptic ulcers that result, for example, from neurosurgery, head injury, severe body trauma or burns. For example, the patient can be administered an effective amount of at least one organic nitric oxide enhancing salt of an NSAID. In another embodiment, the patient can be administered an effective amount of at least one organic nitric oxide enhancing salt of an NSAID, and at least one nitric oxide enhancing compound. In yet another embodiment, the patient can be administered an effective amount of at least one organic nitric oxide enhancing salt of an NSAID, and, at least one therapeutic agent, including but not limited to, such as, for example, steroids, cyclooxygenase-2 (COX-2) inhibitors, nonsteroidal antiinflammatory compounds (NSAID), 5-lipoxygenase (5-LO) inhibitors, leukotriene B₄ (LTB₄) receptor antagonists, leukotriene A₄ (LTA₄) hydrolase inhibitors, 5-HT agonists, anti-hyperlipidemic compounds, H₂ antagonists, antineoplastic agents, antiplatelet agents, thrombin inhibitors, thromboxane inhibitors, carbonic anhydrase inhibitors, decongestants, diuretics, sedating or non-sedating anti-histamines, inducible nitric oxide synthase inhibitors, opioids, analgesics, Helicobacter pylori inhibitors, phosphodiesterase inhibitors, proton pump inhibitors, isoprostane inhibitors, compounds used for the treatment of glaucoma, and combinations of two or more thereof. In another embodiment, the patient can be administered an effective amount of at least one organic nitric oxide enhancing salt of an NSAID, and, at least one therapeutic agent, and, at least one nitric oxide enhancing compound. The organic nitric oxide donor salts of NSAIDs, nitric oxide enhancing compounds, and/or therapeutic agents can be administered separately or as components of the same composition in one or more pharmaceutically acceptable carriers.

Yet another embodiment of the invention provides methods facilitating wound healing (such as, for example, ulcer healing, bone healing including osteoporosis) by administering to the patient in need thereof an effective amount of the compounds and/or compositions described herein. Wound refers to, and includes, any lesion that is characterized by loss of tissue, and, includes, but is not limited to, ulcers, cuts, burns, bone fractures, orthopedic procedure, wound infliction, and the like. Ulcers refers to lesions of the upper gastrointestinal tract lining that are characterized by loss of tissue, and, include, but are not limited to, gastric ulcers, duodenal ulcers, gastritis, and the like. For example, the patient can be administered an effective amount of at least one organic nitric oxide enhancing salt of an NSAID. In another embodiment, the patient can be administered an effective amount of at least one organic nitric oxide enhancing salt of an NSAID, and at least one nitric oxide enhancing compound. In yet another embodiment, the patient can be administered an effective amount of at least one organic nitric oxide enhancing salt of an NSAID, and, at least one therapeutic agent, including but not limited to, such as, for example, steroids, cyclooxygenase-2 (COX-2) inhibitors, nonsteroidal antiinflammatory compounds (NSAID), 5-lipoxygenase (5-LO) inhibitors, leukotriene B₄ (LTB₄) receptor antagonists, leukotriene A4 (LTA₄) hydrolase inhibitors, 5-HT agonists, anti-hyperlipidemic compounds, H₂ antagonists, antineoplastic agents, antiplatelet agents, thrombin inhibitors, thromboxane inhibitors, carbonic anhydrase inhibitors, decongestants, diuretics, sedating or non-sedating anti-histamines, inducible nitric oxide synthase inhibitors, opioids, analgesics, Helicobacter pylori inhibitors, phosphodiesterase inhibitors, proton pump inhibitors, isoprostane inhibitors, compounds used for the treatment of glaucoma, and combinations of two or more thereof. In another embodiment, the patient can be administered an effective amount of at least one organic nitric oxide enhancing salt of an NSAID, and, at least one therapeutic agent, and, at least one nitric oxide enhancing compound. The organic nitric oxide donor salts of NSAIDs, nitric oxide enhancing compounds, and/or therapeutic agents can be administered separately or as components of the same composition in one or more pharmaceutically acceptable carriers.

Another embodiment of the invention provides methods for decreasing and/or reversing gastrointestinal, renal, respiratory and other toxicity (such as, for example, kidney toxicity) resulting from the use of drugs, such as, nonsteroidal anti-inflammatory drugs and/or cyclooxygenase-2 (COX-2) inhibitors by administering to a patient in need thereof an effective amount of the compounds and/or compositions described herein. For example, the patient can be administered an effective amount of at least one organic nitric oxide enhancing salt of an NSAID. In another embodiment, the patient can be administered an effective amount of at least one organic nitric oxide enhancing salt of an NSAID, and at least one nitric oxide enhancing compound. In yet another embodiment, the patient can be administered an effective amount of at least one organic nitric oxide enhancing salt of an NSAID, and, at least one therapeutic agent, including but not limited to, such as, for example, steroids, cyclooxygenase-2 (COX-2) inhibitors, nonsteroidal antiinflammatory compounds (NSAID), 5-lipoxygenase (5-LO) inhibitors, leukotriene B₄ (LTB₄) receptor antagonists, leukotriene A₄ (LTA₄) hydrolase inhibitors, 5-HT agonists, anti-hyperlipidemic compounds, H₂ antagonists, antineoplastic agents, antiplatelet agents, thrombin inhibitors, thromboxane inhibitors, carbonic anhydrase inhibitors, decongestants, diuretics, sedating or non-sedating anti-histamines, inducible nitric oxide synthase inhibitors, opioids, analgesics, Helicobacter pylori inhibitors, phosphodiesterase inhibitors, proton pump inhibitors, isoprostane inhibitors, compounds used for the treatment of glaucoma, and combinations of two or more thereof. In another embodiment, the patient can be administered an effective amount of at least one organic nitric oxide enhancing salt of an NSAID, and, at least one therapeutic agent, and, at least one nitric oxide enhancing compound. The organic nitric oxide donor salts of NSAIDs, nitric oxide enhancing compounds, and/or therapeutic agents can be administered separately or as components of the same composition in one or more pharmaceutically acceptable carriers.

Another embodiment of the invention provides for treating inflammatory disease states and disorders by administering to a patient in need thereof an effective amount of the compounds and/or compositions described herein. Such inflammatory disease states and/or disorders include, for example, cardiovascular disorder, reperfusion injury to an ischemic organ, angiogenisis, arthritis, including but not limited to rheumatoid arthritis, degenerative joint disease (osteoarthritis), spondyloarthropathies, gouty arthritis, systemic lupus erythematosus and juvenile arthritis; asthma, bronchitis, premature labor, tendinitis, bursitis; autoimmune diseases, immunological disorders; skin-related conditions, such as, for example, psoriasis, eczema, surface wounds, burns and dermatitis; post-operative inflammation including from ophthalmic surgery, such as, for example, cataract surgery and refractive surgery, and the like; neoplasia, such as, for example, brain cancer, bone cancer, epithelial cell-derived neoplasia (epithelial carcinoma), such as, for example, basal cell carcinoma, adenocarcinoma, gastrointestinal cancer, such as, for example, lip cancer, mouth cancer, esophageal cancer, small bowel cancer and stomach cancer, colon cancer, liver cancer, bladder cancer, pancreas cancer, ovary cancer, cervical cancer, lung cancer, breast cancer and skin cancer, such as squamus cell and basal cell cancers, prostate cancer, renal cell carcinoma, and other known cancers that effect epithelial cells throughout the body, benign and cancerous tumors, growths, polyps, adenomatous polyps, including, but not limited to, familial adenomatous polyposis, fibrosis resulting from radiation therapy, and the like; inflammatory processes in diseases, such as, for example, vascular diseases, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury, myocardial ischemia, and the like; pulmonary inflammation, such as, for example, those associated with viral infections and cystic fibrosis, and the like; central nervous system disorders, such as, for example, cortical dementia including Alzheimer's disease, vascular dementia, multi-infarct dementia, pre-senile dementia, alcoholic dementia, senile dementia, memory loss and central nervous system damage resulting from stroke, ischemia and trauma, and the like; allergic rhinitis, respiratory distress syndrome, endotoxin shock syndrome, inflammations and/or microbial infections including, for example, inflammations and/or infections of the eyes, ears, nose, throat, and/or skin; bacterial-induced inflammation, such as, for example, Chlamydia-induced inflammation; viral induced inflammation, urinary and/or urological disorders, such as, for example, incontinence and the like; endothelial dysfunctions, such as, for example, diseases accompanying these dysfunctions, endothelial damage from hypercholesterolemia, endothelial damage from hypoxia, endothelial damage from mechanical and chemical noxae, especially during and after drug, and mechanical reopening of stenosed vessels, for example, following percutaneous transluminal angiography (PTA) and percutaneous transluminal coronary angiography (PTCA), endothelial damage in postinfarction phase, endothelium-mediated reocculusion following bypass surgery, blood supply disturbances in peripheral arteries, and the like; sexual dysfunction; tissue deterioration, such as, for example, for organ transplant rejection, and the like; disorders treated by the inhibition and/or prevention of activation, adhesion and infiltration of neutrophils at the site of inflammation; and disorders treated by the inhibition and/or prevention of platelet aggregation. The compounds and compositions of the invention can also be used as a pre-anesthetic medication in emergency operations to reduce the danger of aspiration of acidic gastric contents. For example, the patient can be administered an effective amount of at least one organic nitric oxide enhancing salt of an NSAID. In another embodiment, the patient can be administered an effective amount of at least one organic nitric oxide enhancing salt of an NSAID, and at least one nitric oxide enhancing compound. In yet another embodiment, the patient can be administered an effective amount of at least one organic nitric oxide enhancing salt of an NSAID, and, at least one therapeutic agent, including but not limited to, such as, for example, steroids, cyclooxygenase-2 (COX-2) inhibitors, nonsteroidal antiinflammatory compounds (NSAID), 5-lipoxygenase (5-LO) inhibitors, leukotriene B₄ (LTB₄) receptor antagonists, leukotriene A₄ (LTA₄) hydrolase inhibitors, 5-HT agonists, anti-hyperlipidemic compounds, H₂ antagonists, antineoplastic agents, antiplatelet agents, thrombin inhibitors, thromboxane inhibitors, carbonic anhydrase inhibitors, decongestants, diuretics, sedating or non-sedating anti-histamines, inducible nitric oxide synthase inhibitors, opioids, analgesics, Helicobacter pylori inhibitors, phosphodiesterase inhibitors, proton pump inhibitors, isoprostane inhibitors, compounds used for the treatment of glaucoma, and combinations of two or more thereof. In another embodiment, the patient can be administered an effective amount of at least one organic nitric oxide enhancing salt of an NSAID, and, at least one therapeutic agent, and, at least one nitric oxide enhancing compound. The organic nitric oxide donor salts of NSAIDs, nitric oxide enhancing compounds, and/or therapeutic agents can be administered separately or as components of the same composition in one or more pharmaceutically acceptable carriers.

Another embodiment of the invention provides methods for treating ophthalmic disorders; treating peripheral vascular diseases; treating diseases resulting from oxidative stress; treating endothelial dysfunctions; and treating diseases caused by endothelial dysfunctions by administering to a patient in need thereof an effective amount of the compounds and/or compositions described herein. For example, the patient can be administered an effective amount of at least one organic nitric oxide enhancing salt of an NSAID. In another embodiment, the patient can be administered an effective amount of at least one organic nitric oxide enhancing salt of an NSAID, and at least one nitric oxide enhancing compound. In yet another embodiment, the patient can be administered an effective amount of at least one organic nitric oxide enhancing salt of an NSAID, and, at least one therapeutic agent, including but not limited to, such as, for example, steroids, cyclooxygenase-2 (COX-2) inhibitors, nonsteroidal antiinflammatory compounds (NSAID), 5-lipoxygenase (5-LO) inhibitors, leukotriene B₄ (LTB₄) receptor antagonists, leukotriene A₄ (LTA₄) hydrolase inhibitors, 5-HT agonists, anti-hyperlipidemic compounds, H₂ antagonists, antineoplastic agents, antiplatelet agents, thrombin inhibitors, thromboxane inhibitors, carbonic anhydrase inhibitors, decongestants, diuretics, sedating or non-sedating anti-histamines, inducible nitric oxide synthase inhibitors, opioids, analgesics, Helicobacter pylori inhibitors, phosphodiesterase inhibitors, proton pump inhibitors, isoprostane inhibitors, compounds used for the treatment of glaucoma, and combinations of two or more thereof. In another embodiment, the patient can be administered an effective amount of at least one organic nitric oxide enhancing salt of an NSAID, and, at least one therapeutic agent, and, at least one nitric oxide enhancing compound. The organic nitric oxide donor salts of NSAIDs, nitric oxide enhancing compounds, and/or therapeutic agents can be administered separately or as components of the same composition in one or more pharmaceutically acceptable carriers.

When administered separately, the organic nitric oxide enhancing salt of a NSAID, nitric oxide enhancing compound and/or therapeutic agent can be administered about the same time as part of the overall treatment regimen, i.e., as a combination therapy. “About the same time” includes administering the NSAID compound comprising at least one nitric oxide enhancing group, simultaneously, sequentially, at the same time, at different times on the same day, or on different days, as long as they are administered as part of an overall treatment regimen, i.e., combination therapy or a therapeutic cocktail.

When administered in vivo, the compounds and compositions of the invention can be administered in combination with pharmaceutically acceptable carriers and in dosages described herein. When the compounds and compositions of the invention are administered as a combination of at least one organic nitric oxide enhancing salt of a NSAID and/or at least one nitric oxide enhancing compound and/or therapeutic agent, they can also be used in combination with one or more additional compounds which are known to be effective against the specific disease state targeted for treatment. The nitric oxide enhancing compounds, therapeutic agents and/or other additional compounds can be administered simultaneously with, subsequently to, or prior to administration of the organic nitric oxide enhancing salt of the NSAID.

The compounds and compositions of the invention can be administered by any available and effective delivery system including, but not limited to, orally, bucally, parenterally, by inhalation, by topical application, by injection, transdermally, or rectally (e.g., by the use of suppositories) in dosage unit formulations containing conventional nontoxic pharmaceutically acceptable carriers, adjuvants, and vehicles, as desired. Parenteral includes subcutaneous injections, intravenous, intramuscular, intrasternal injection, or infusion techniques. In one embodiment of the invention the organic nitric oxide enhancing salt of the NSAID is administered orally, parentally or by inhalation.

Transdermal compound administration, which is known to one skilled in the art, involves the deli Transdermal compound administration, which is known to one skilled in the art, involves the delivery of pharmaceutical compounds via percutaneous passage of the compound into the systemic circulation of the patient. Topical administration can also involve the use of transdermal administration such as transdermal patches or iontophoresis devices. Other components can be incorporated into the transdermal patches as well. For example, compositions and/or transdermal patches can be formulated with one or more preservatives or bacteriostatic agents including, but not limited to, methyl hydroxybenzoate, propyl hydroxybenzoate, chlorocresol, benzalkonium chloride, and the like. Dosage forms for topical administration of the compounds and compositions can include creams, sprays, lotions, gels, ointments, eye drops, nose drops, ear drops, and the like. In such dosage forms, the compositions of the invention can be mixed to form white, smooth, homogeneous, opaque cream or lotion with, for example, benzyl alcohol 1% or 2% (wt/wt) as a preservative, emulsifying wax, glycerin, isopropyl palmitate, lactic acid, purified water and sorbitol solution. In addition, the compositions can contain polyethylene glycol 400. They can be mixed to form ointments with, for example, benzyl alcohol 2% (wt/wt) as preservative, white petrolatum, emulsifying wax, and tenox II (butylated hydroxyanisole, propyl gallate, citric acid, propylene glycol). Woven pads or rolls of bandaging material, e.g., gauze, can be impregnated with the compositions in solution, lotion, cream, ointment or other such form can also be used for topical application. The compositions can also be applied topically using a transdermal system, such as one of an acrylic-based polymer adhesive with a resinous crosslinking agent impregnated with the composition and laminated to an impermeable backing.

The compositions can also be applied topically using a transdermal system, such as one of an acrylic-based polymer adhesive with a resinous crosslinking agent impregnated with the composition and laminated to an impermeable backing. In a particular embodiment, the compositions of the invention are administered as a transdermal patch, more particularly as a sustained-release transdermal patch. The transdermal patches of the invention can include any conventional form such as, for example, adhesive matrix, polymeric matrix, reservoir patch, matrix or monolithic-type laminated structure, and are generally comprised of one or more backing layers, adhesives, penetration enhancers, an optional rate controlling membrane and a release liner which is removed to expose the adhesives prior to application. Polymeric matrix patches also comprise a polymeric-matrix forming material. Suitable transdermal patches are described in more detail in, for example, U.S. Pat. Nos. 5,262,165, 5,948,433, 6,010,715 and 6,071,531, the disclosure of each of which are incorporated herein in their entirety.

Solid dosage forms for oral administration can include capsules, sustained-release capsules, tablets, sustained release tablets, chewable tablets, sublingual tablets, effervescent tablets, pills, powders, granules and gels. In such solid dosage forms, the active compounds can be admixed with at least one inert diluent such as sucrose, lactose or starch. Such dosage forms can also comprise, as in normal practice, additional substances other than inert diluents, e.g., lubricating agents such as magnesium stearate. In the case of capsules, tablets, effervescent tablets, and pills, the dosage forms can also comprise buffering agents. Soft gelatin capsules can be prepared to contain a mixture of the active compounds or compositions of the invention and vegetable oil. Hard gelatin capsules can contain granules of the active compound in combination with a solid, pulverulent carrier such as lactose, saccharose, sorbitol, mannitol, potato starch, corn starch, amylopectin, cellulose derivatives of gelatin. Tablets and pills can be prepared with enteric coatings.

Liquid dosage forms for oral administration can include pharmaceutically acceptable emulsions, solutions, suspensions, syrups, and elixirs containing inert diluents commonly used in the art, such as water. Such compositions can also comprise adjuvants, such as wetting agents, emulsifying and suspending agents, and sweetening, flavoring, and perfuming agents.

Suppositories for vaginal or rectal administration of the compounds and compositions of the invention can be prepared by mixing the compounds or compositions with a suitable nonirritating excipient such as cocoa butter and polyethylene glycols which are solid at room temperature but liquid at rectal temperature, such that they will melt in the rectum and release the drug.

Injectable preparations, for example, sterile injectable aqueous or oleaginous suspensions can be formulated according to the known art using suitable dispersing agents, wetting agents and/or suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. Sterile fixed oils are also conventionally used as a solvent or suspending medium.

The compositions of this invention can further include conventional excipients, i.e., pharmaceutically acceptable organic or inorganic carrier substances suitable for parenteral application which do not deleteriously react with the active compounds. Suitable pharmaceutically acceptable carriers include, for example, water, salt solutions, alcohol, vegetable oils, polyethylene glycols, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like. The pharmaceutical preparations can be sterilized and if desired, mixed with auxiliary agents, e.g., lubricants, preservatives, stabilizers, wetting agents, emulsifiers, salts for influencing osmotic pressure, buffers, colorings, flavoring and/or aromatic substances and the like which do not deleteriously react with the active compounds. For parenteral application, particularly suitable vehicles consist of solutions, preferably oily or aqueous solutions, as well as suspensions, emulsions, or implants. Aqueous suspensions may contain substances which increase the viscosity of the suspension and include, for example, sodium carboxymethyl cellulose, sorbitol and/or dextran. Optionally, the suspension may also contain stabilizers.

The composition, if desired, can also contain minor amounts of wetting agents, emulsifying agents and/or pH buffering agents. The composition can be a liquid solution, suspension, emulsion, tablet, pill, capsule, sustained release formulation, or powder. The composition can be formulated as a suppository, with traditional binders and carriers such as triglycerides. Oral formulations can include standard carriers such as pharmaceutical grades of mannitol, lactose, starch, magnesium stearate, sodium saccharine, cellulose, magnesium carbonate, and the like.

Various delivery systems are known and can be used to administer the compounds or compositions of the invention, including, for example, encapsulation in liposomes, microbubbles, emulsions, microparticles, microcapsules and the like. The required dosage can be administered as a single unit or in a sustained release form.

The bioavailability of the compositions can be enhanced by micronization of the formulations using conventional techniques such as grinding, milling, spray drying and the like in the presence of suitable excipients or agents such as phospholipids or surfactants. Sustained release dosage forms of the invention may comprise microparticles and/or nanoparticles having a therapeutic agent dispersed therein or may comprise the therapeutic agent in pure, preferably crystalline, solid form. For sustained release administration, microparticle dosage forms comprising pure, preferably crystalline, therapeutic agents are preferred. The therapeutic dosage forms of this aspect of the invention may be of any configuration suitable for sustained release.

Nanoparticle sustained release therapeutic dosage forms are preferably biodegradable and, optionally, bind to the vascular smooth muscle cells and enter those cells, primarily by endocytosis. The biodegradation of the nanoparticles occurs over time (e.g., 30 to 120 days; or 10 to 21 days) in prelysosomic vesicles and lysosomes. Preferred larger microparticle therapeutic dosage forms of the invention release the therapeutic agents for subsequent target cell uptake with only a few of the smaller microparticles entering the cell by phagocytosis. A practitioner in the art will appreciate that the precise mechanism by which a target cell assimilates and metabolizes a dosage form of the invention depends on the morphology, physiology and metabolic processes of those cells. The size of the particle sustained release therapeutic dosage forms is also important with respect to the mode of cellular assimilation. For example, the smaller nanoparticles can flow with the interstitial fluid between cells and penetrate the infused tissue. The larger microparticles tend to be more easily trapped interstitially in the infused primary tissue, and thus are useful to deliver anti-proliferative therapeutic agents.

Particular sustained release dosage forms of the invention comprise biodegradable microparticles or nanoparticles. More particularly, biodegradable microparticles or nanoparticles are formed of a polymer containing matrix that biodegrades by random, nonenzymatic, hydrolytic scissioning to release therapeutic agent, thereby forming pores within the particulate structure.

In a particular embodiment, the compositions of the invention are orally administered as a sustained release tablet or a sustained release capsule. For example, the sustained release formulations can comprise an effective amount of at least one organic nitric oxide enhancing salt of an NSAID, and, optionally at least one nitric oxide enhancing compound or the sustained release formulations can comprise an effective amount of at least one organic nitric oxide enhancing salt of an NSAID, and at least one nitric oxide enhancing compound, and, optionally at least one therapeutic agent

While individual needs may vary, determination of optimal ranges for effective amounts of the compounds and/or compositions is within the skill of the art. Generally, the dosage required to provide an effective amount of the compounds and compositions, which can be adjusted by one of ordinary skill in the art, will vary depending on the age, health, physical condition, sex, diet, weight, extent of the dysfunction of the recipient, frequency of treatment and the nature and scope of the dysfunction or disease, medical condition of the patient, the route of administration, pharmacological considerations such as the activity, efficacy, pharmacokinetic and toxicology profiles of the particular compound used, whether a drug delivery system is used, and whether the compound is administered as part of a drug combination.

The amount of a given organic nitric oxide enhancing salt of an NSAID that will be effective in the treatment of a particular disorder or condition will depend on the nature of the disorder or condition, and can be determined by standard clinical techniques, including reference to Goodman and Gilman, supra; The Physician's Desk Reference, Medical Economics Company, Inc., Oradell, N.J., 1995; and Drug Facts and Comparisons, Inc., St. Louis, Mo., 1993. The precise dose to be used in the formulation will also depend on the route of administration, and the seriousness of the disease or disorder, and should be decided by the physician and the patient's circumstances.

The invention also provides pharmaceutical kits comprising one or more containers filled with one or more of the ingredients of the pharmaceutical compounds and/or compositions of the invention, including, at least, one or more of the organic nitric oxide enhancing salt of the NSAIDs, and one or more of the nitric oxide enhancing compounds described herein. Associated with such kits can be additional therapeutic agents or compositions ((e.g., steroids, COX-2 inhibitors, nonsteroidal antiinflammatory compounds (NSAID), 5-lipoxygenase (5-LO) inhibitors, leukotriene B₄ (LTB₄) receptor antagonists and leukotriene A₄ (LTA₄) hydrolase inhibitors, 5-HT agonists, anti-hyperlipidemic compounds, H₂ antagonists, antineoplastic agents, antiplatelet agents, thrombin inhibitors, thromboxane inhibitors, carbonic anhydrase inhibitors, decongestants, diuretics, sedating or non-sedating anti-histamines, inducible nitric oxide synthase inhibitors, opioids, analgesics, Helicobacter pylori inhibitors, phosphodiesterase inhibitors, proton pump inhibitors, isoprostane inhibitors, compounds used for the treatment of glaucoma, and the like, and combinations of two or more thereof), devices for administering the compositions, and notices in the form prescribed by a governmental agency regulating the manufacture, use or sale of pharmaceuticals or biological products which reflects approval by the agency of manufacture, use or sale for humans.

The disclosure of each patent, patent application and publication cited or described in the present specification is hereby incorporated by reference herein in its entirety.

Although the invention has been set forth in detail, one skilled in the art will appreciate that numerous changes and modifications can be made to the invention, and that such changes and modifications can be made without departing from the spirit and scope of the invention.

What is claimed is:

1. A compound of Formula (I) or (II):
wherein the compound of Formula (I) is: 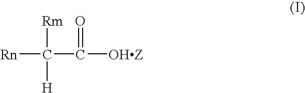
wherein:
Rₘ is a hydrogen or a lower alkyl group;
Rₙ is: 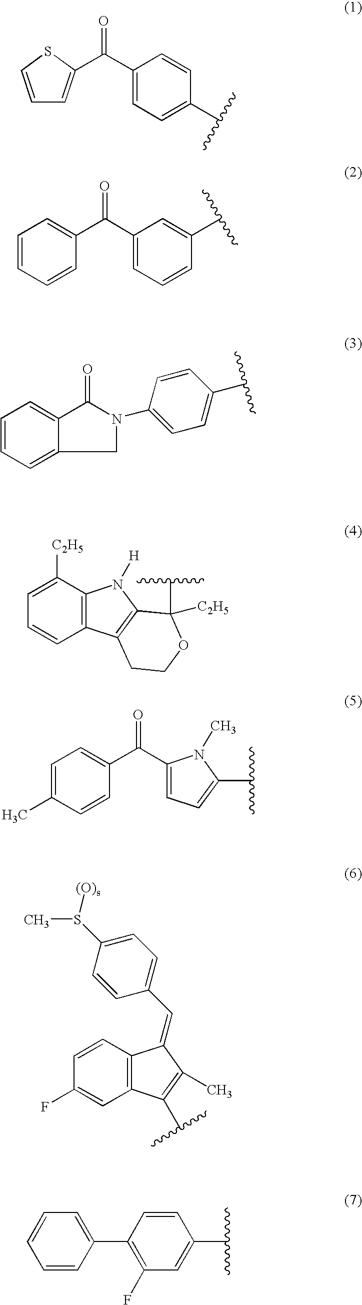 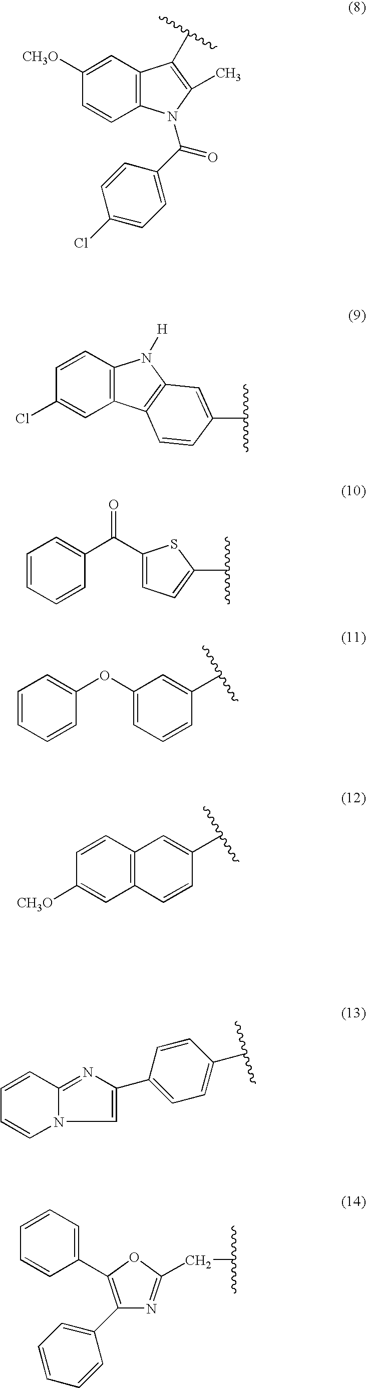 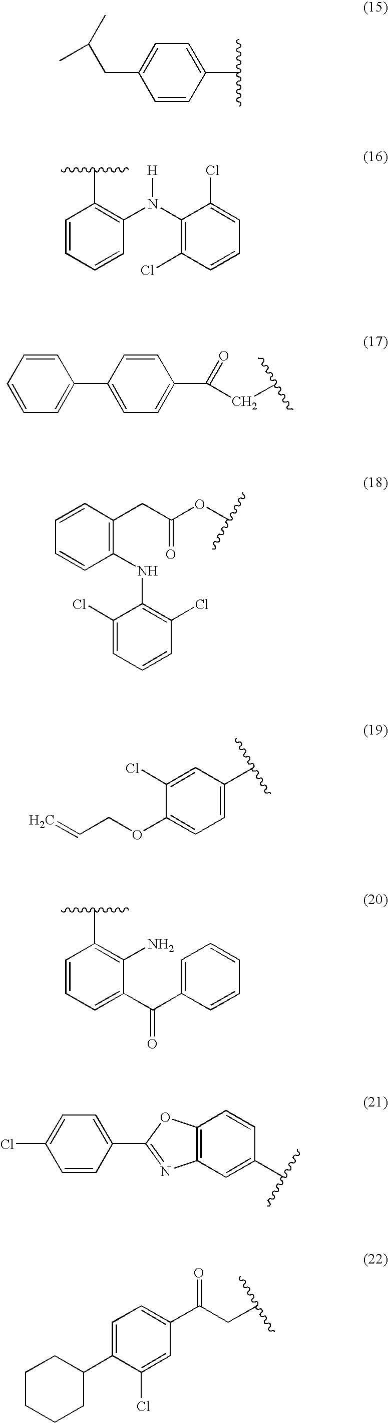 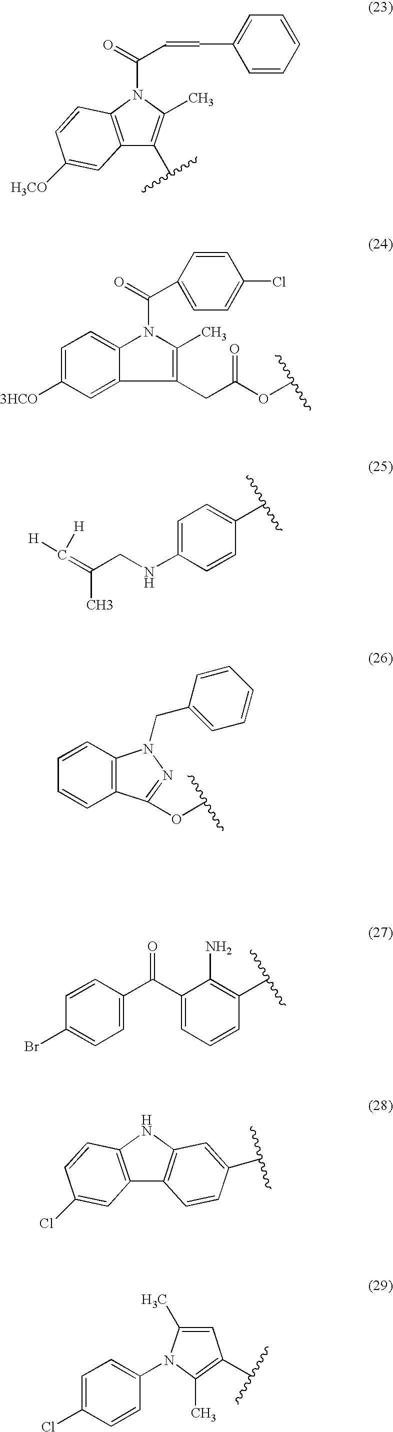 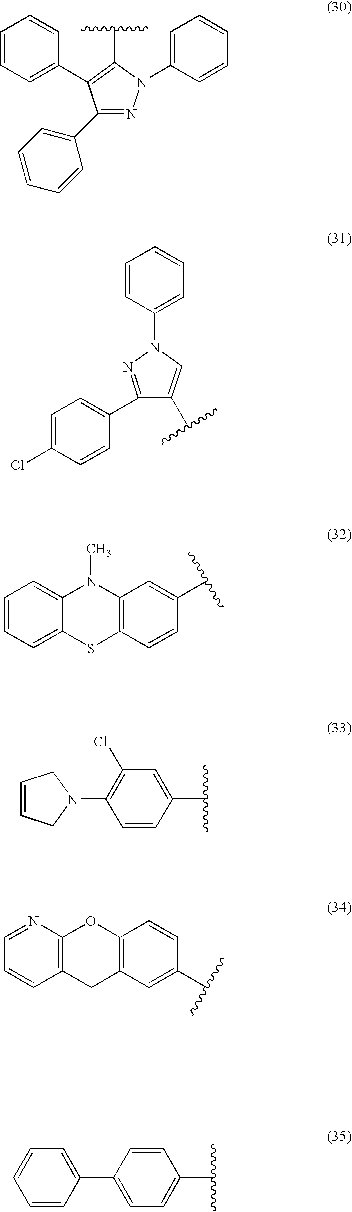 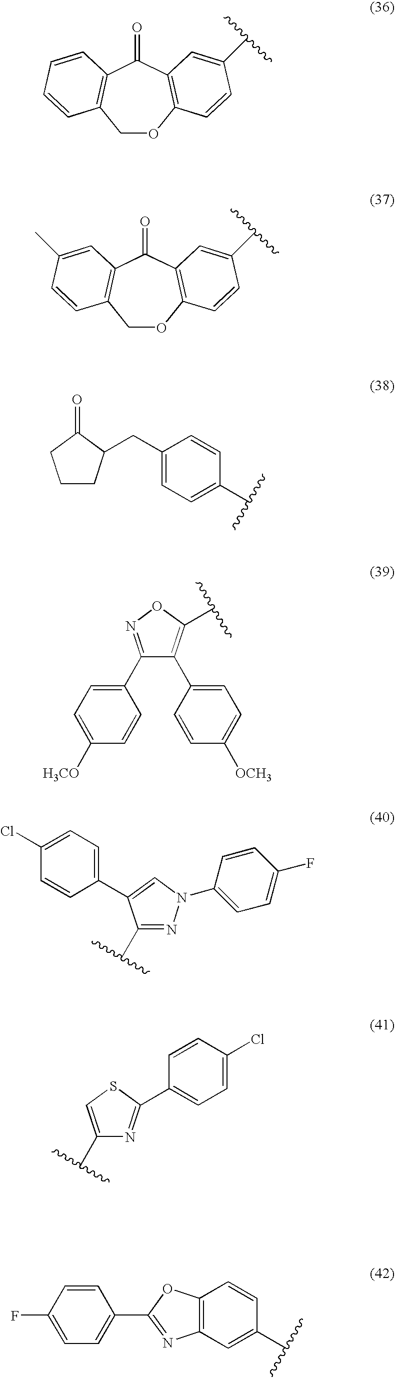 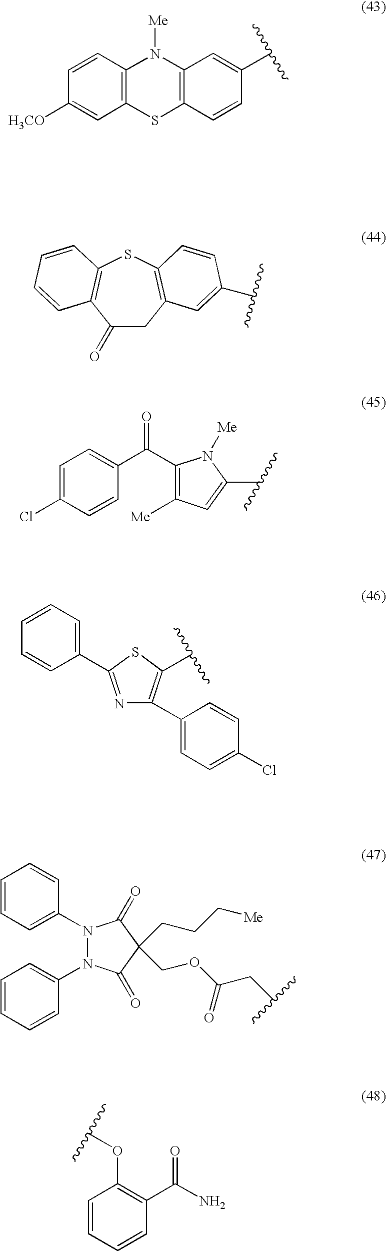 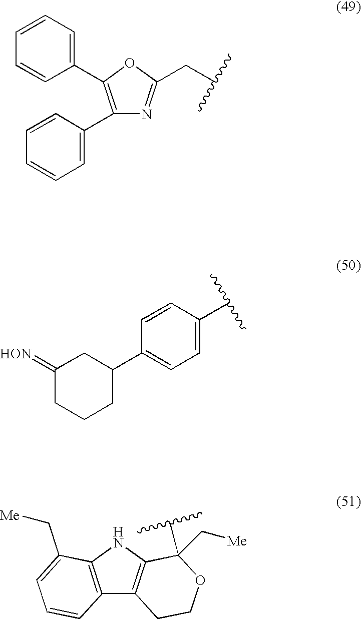
s is an integer of 0 or 1;
Z is an organic base or —N(R₃₈)(R₃₉)(R₄₀);
R₃₈, R₃₉ and R₄₀ are each independently selected from K or Rₑ, or R₃₈ and R₃₉ taken together with the nitrogen to which they are attached are a heterocyclic ring, with the proviso that when the heterocyclic ring is an aromatic ring it can be substituted at any position by L and R₃₉ is not present;
L is —(W₃)ₐ-E_{b}-(C(Rₑ)(R_{f}))ₚ₁-E_{c}-(C(Rₑ)(R_{f}))ₓ—(W₃)_{d}—(C(Rₑ)(R_{f}))_{y}—(W₃)ᵢ-Eⱼ-(W₃)_{g}—(C(Rₑ)(R_{f}))_{z}—V₄;
K is —(W₃)ₐ-E_{b}-(C(Rₑ)(R_{f}))ₚ₁-E_{c}-(C(Rₑ)(R_{f}))ₓ—(W₃)_{d}—(C(Rₑ)(R_{f}))_{y}—(W₃)ᵢ-Eⱼ-(W₃)_{g}—(C(Rₑ)(R_{f}))_{z}—V₄;
a, b, c, d, g, i and j are each independently an integer from 0 to 3;
p₁, x, y and z are each independently an integer from 0 to 10;
V₄ is V₃, Rₑ, —U₃—V₅ or V₆;
V₃ is: 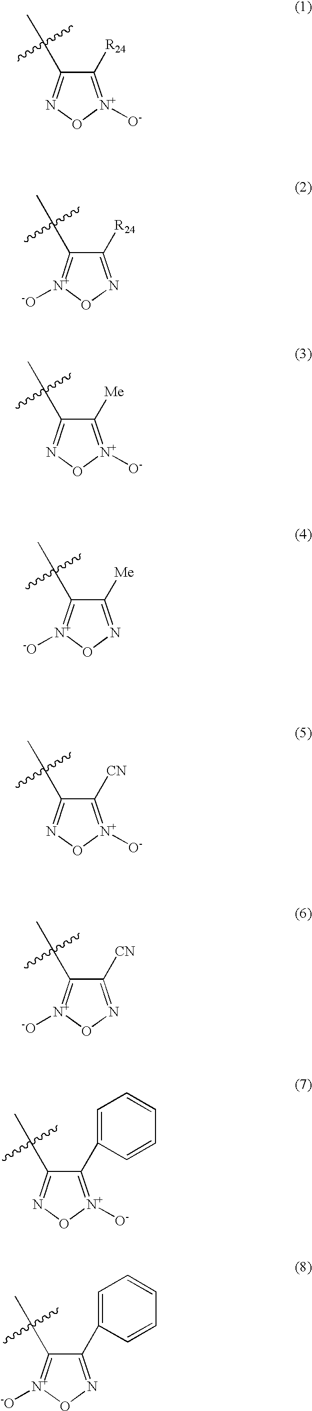 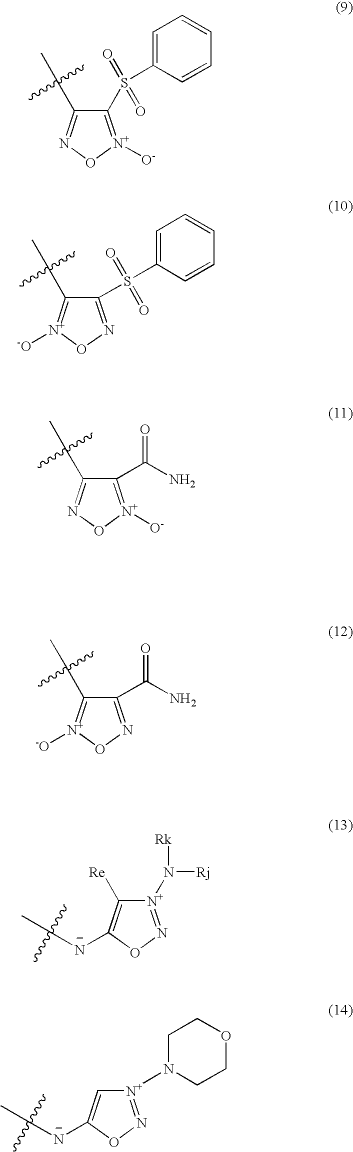 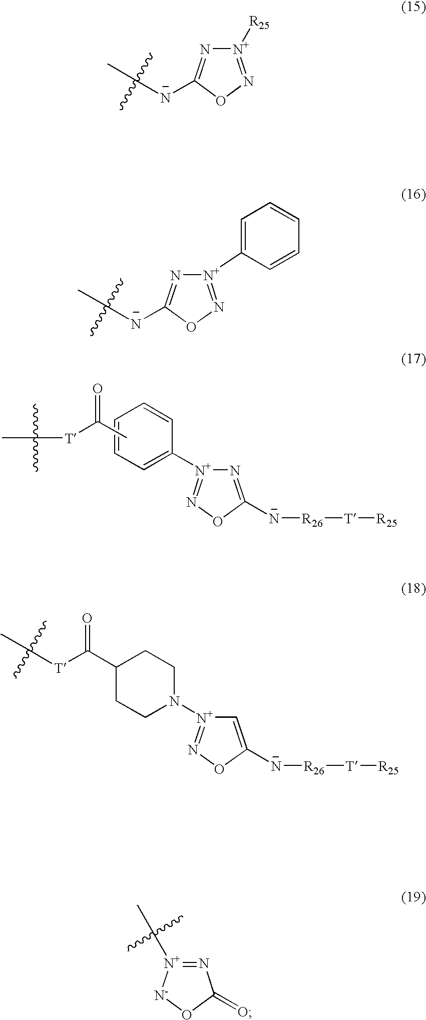
R₂₄ is —C₆H₄R₃₇, —CN, —S(O)₂—C₆H₄R₃₇, —C(O)—N(Rₐ)(Rᵢ), —NO₂, —C(O)—OR₂₅ or —S(O)₂—R₂₅;
R₂₅ is an aryl group, a lower alkyl group, a haloalkyl group, a hydroxyalkyl group or an arylalkyl group;
R₂₆ is —C(O)— or —S(O)₂—;
R₃₇ is a hydrogen, —CN, —S(O)₂—R₂₅, —C(O)—N(Rₐ)(Rᵢ), —NO₂ or —C(O)—OR₂₅;
T′ is oxygen, sulfur or NR₁₆;
R₁₆ is a hydrogen, a lower alkyl group, or an aryl group;
V₆ is: 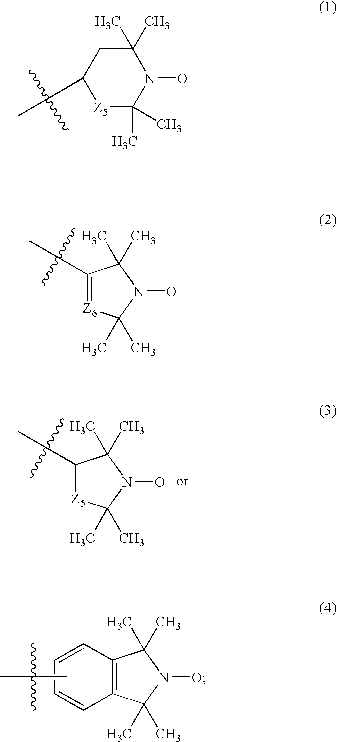
Z₅ is —CH₂ or oxygen;
Z₆ is —CH or nitrogen;
W₃ at each occurrence is independently —C(O)—, —C(S)—, -T₃-, —(C(Rₑ)(R_{f}))ₕ—, —N(Rₐ)Rᵢ, an alkyl group, an aryl group, a heterocyclic ring, an arylheterocyclic ring, —(CH₂CH₂O)_{q1}— or a heterocyclic nitric oxide donor;
E at each occurrence is independently -T₃-, an alkyl group, an aryl group, —(C(Rₑ)(R_{f}))ₕ—, a heterocyclic ring, an arylheterocyclic ring, —(CH₂CH₂O)_{q1}— or Y₄;
Y₄ is: 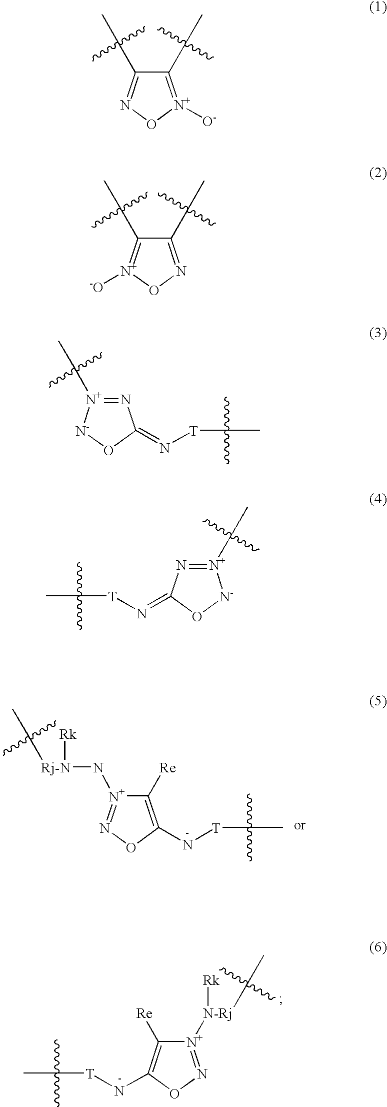
T is a —S(O)ₒ—; a carbonyl or a covalent bond;
o is an integer from 0 to 2;
Rⱼ and Rₖ are independently selected from an alkyl group, an aryl group, or Rⱼ and Rₖ taken together with the nitrogen atom to which they are attached are a heterocylic ring;
T₃ at each occurrence is independently a covalent bond, a carbonyl, an oxygen, —S(O)ₒ— or —N(Rₐ)Rᵢ;
h is an integer form 1 to 10;
q₁ is an integer from 1 to 5;
Rₑ and R_{f} are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl, a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano, an aminoalkyl, an aminoaryl, an aryl, an arylalkyl, an alkylaryl, a carboxamido, an alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, —U₃—V₅, V₆, —(C(Rₒ)(Rₚ))ₖ₁—U₃—V₅, —(C(Rₒ)(Rₚ))ₖ₁—U₃—V₃, —(C(Rₒ)(Rₚ))ₖ₁—U₃—V₆, —(C(Rₒ)(Rₚ))ₖ₁—U₃—C(O)—V₆, or Rₑ and R_{f} taken together with the carbons to which they are attached form a carbonyl, a methanthial, a heterocyclic ring, a cycloalkyl group, an aryl group, an oxime, an imine, a hydrazone, a bridged cycloalkyl group, 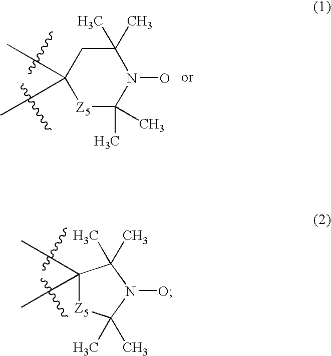
Rₒ and Rₚ are each independently a hydrogen, an alkyl, a cycloalkoxy, a halogen, a hydroxy, an hydroxyalkyl, an alkoxyalkyl, an arylheterocyclic ring, an alkylaryl, an alkylcycloalkyl, an alkylheterocyclic ring, a cycloalkylalkyl, a cycloalkylthio, an arylalklythio, an arylalklythioalkyl, an alkylthioalkyl a cycloalkenyl, an heterocyclicalkyl, an alkoxy, a haloalkoxy, an amino, an alkylamino, a dialkylamino, an arylamino, a diarylamino, an alkylarylamino, an alkoxyhaloalkyl, a sulfonic acid, a sulfonic ester, an alkylsulfonic acid, an arylsulfonic acid, an arylalkoxy, an alkylthio, an arylthio, a cyano an aminoalkyl, an aminoaryl, an aryl, an arylalkyl, an alkylaryl, a carboxamido, an alkylcarboxamido, an arylcarboxamido, an amidyl, a carboxyl, a carbamoyl, an alkylcarboxylic acid, an arylcarboxylic acid, an alkylcarbonyl, an arylcarbonyl, an ester, a carboxylic ester, an alkylcarboxylic ester, an arylcarboxylic ester, a sulfonamido, an alkylsulfonamido, an arylsulfonamido, an alkylsulfonyl, an alkylsulfonyloxy, an arylsulfonyl, arylsulphonyloxy, a sulfonic ester, an alkyl ester, an aryl ester, a urea, a phosphoryl, a nitro, —U₃—V₅, V₆, or Rₒ and Rₚ taken together with the carbons to which they are attached form a carbonyl, a methanthial, a heterocyclic ring, a cycloalkyl group, an aryl group, an oxime, an imine, a hydrazone a bridged cycloalkyl group, 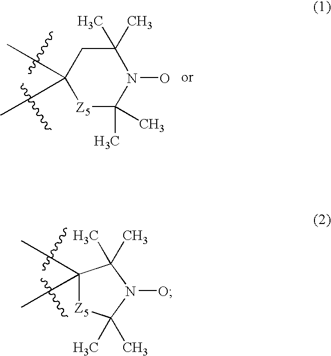
U₃ is an oxygen, sulfur or —N(Rₐ)Rᵢ;
V₅ is —NO or —NO₂ (i.e. an oxidized nitrogen);
k₁ is an integer from 1 to 3;
Rₐ is a lone pair of electrons, a hydrogen or an alkyl group;
Rᵢ is a hydrogen, an alkyl, an aryl, an alkylcarboxylic acid, an arylcarboxylic acid, an alkylcarboxylic ester, an arylcarboxylic ester, an alkylcarboxamido, an arylcarboxamido, an alkylaryl, an alkylsulfinyl, an alkylsulfonyl, an alkylsulfonyloxy, an arylsulfinyl, an arylsulfonyl, an arylsulphonyloxy, a sulfonamido, a carboxamido, a carboxylic ester, an aminoalkyl, an aminoaryl, —CH₂—C—(U₃—V₅)(Rₑ)(R_{f}), a bond to an adjacent atom creating a double bond to that atom or —(N₂O₂—).M₁⁺, wherein M₁⁺ is an organic or inorganic cation; and
with the proviso that the compound of Formula (I) must contain at least one organic nitric oxide enhancing compound linked via a salt bridge (i.e., .) to at least one carboxylic acid group;
wherein the compound of Formula (II): 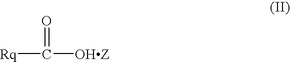
wherein:
R_{q} is: 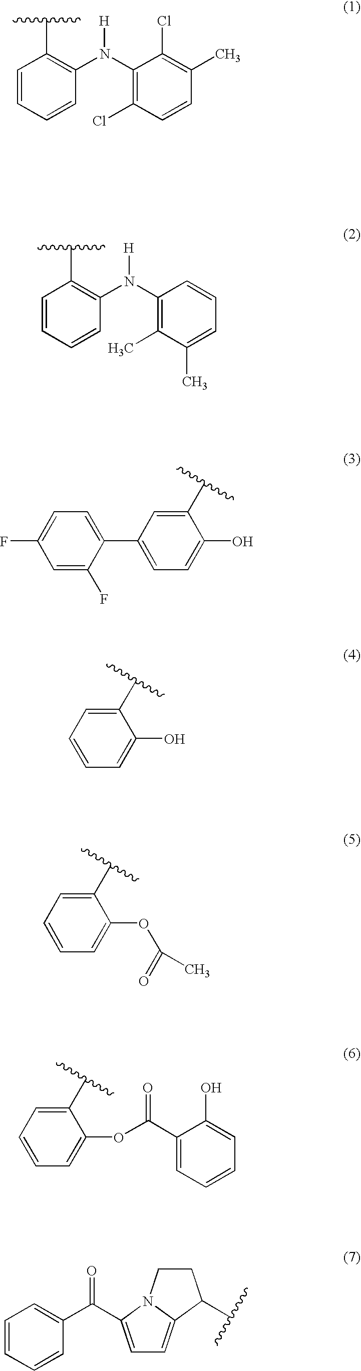 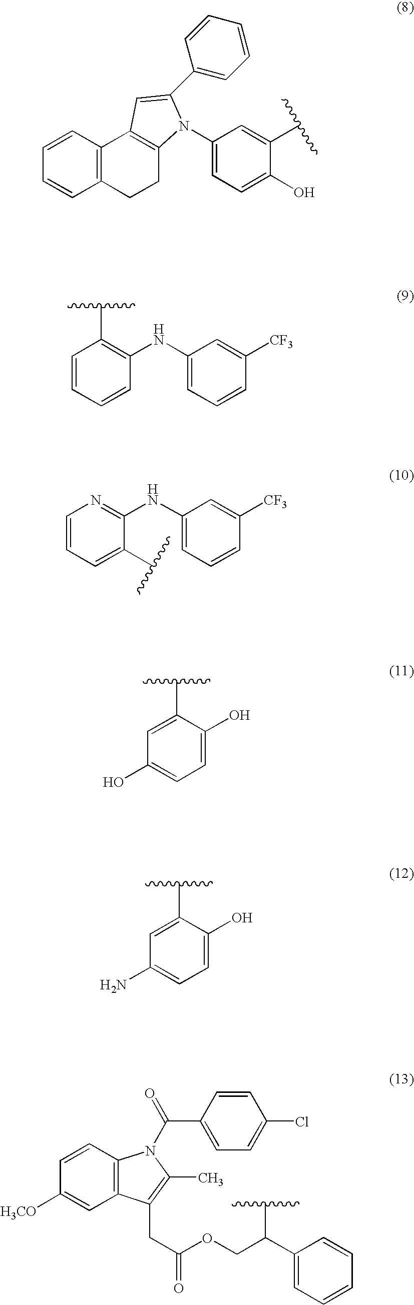 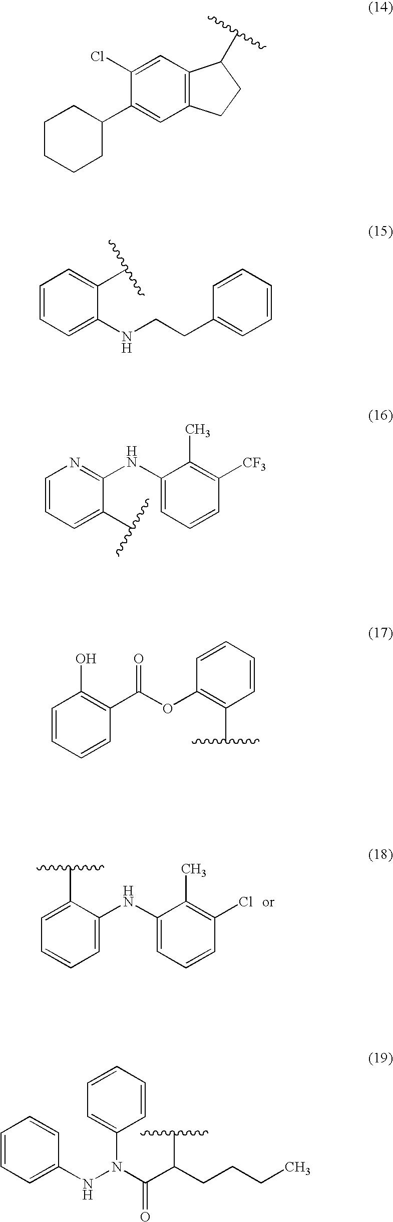
and Z is as defined herein; and
with the proviso that the compounds of Formula (II) must contain at least one organic nitric oxide enhancing compound linked via a salt bridge (i.e., .) to at least one carboxylic acid group.

2. A composition comprising the compound of claim 1 and a pharmaceutically acceptable carrier.

3. The compound of claim 1, wherein the compound of Formula (I) is an organic nitric oxide enhancing salt of acemetacin, an organic nitric oxide enhancing salt of aceclofenac, an organic nitric oxide enhancing salt of alclofenac, an organic nitric oxide enhancing salt of alminoprofen, an organic nitric oxide enhancing salt of amfenac, an organic nitric oxide enhancing salt of bendazac, an organic nitric oxide enhancing salt of benoxaprofen, an organic nitric oxide enhancing salt of bromfenac, an organic nitric oxide enhancing salt of bucloxic acid, an organic nitric oxide enhancing salt of butibufen, an organic nitric oxide enhancing salt of carprofen, an organic nitric oxide enhancing salt of cinmetacin, an organic nitric oxide enhancing salt of clopirac, an organic nitric oxide enhancing salt of diclofenac, an organic nitric oxide enhancing salt of etodolac, an organic nitric oxide enhancing salt of felbinac, an organic nitric oxide enhancing salt of fenclozic acid, an organic nitric oxide enhancing salt of fenbufen, an organic nitric oxide enhancing salt of fenoprofen, an organic nitric oxide enhancing salt of fentiazac, an organic nitric oxide enhancing salt of flunoxaprofen, an organic nitric oxide enhancing salt of flurbiprofen, an organic nitric oxide enhancing salt of ibufenac, an organic nitric oxide enhancing salt of ibuprofen, an organic nitric oxide enhancing salt of indomethacin, an organic nitric oxide enhancing salt of isofezolac, an organic nitric oxide enhancing salt of isoxepac, an organic nitric oxide enhancing salt of indoprofen, an organic nitric oxide enhancing salt of ketoprofen, an organic nitric oxide enhancing salt of lonazolac, an organic nitric oxide enhancing salt of loxoprofen, an organic nitric oxide enhancing salt of metiazinic acid, an organic nitric oxide enhancing salt of mofezolac, an organic nitric oxide enhancing salt of miroprofen, an organic nitric oxide enhancing salt of naproxen, an organic nitric oxide enhancing salt of oxaprozin, an organic nitric oxide enhancing salt of pirozolac, an organic nitric oxide enhancing salt of pirprofen, an organic nitric oxide enhancing salt of pranoprofen, an organic nitric oxide enhancing salt of protizinic acid, an organic nitric oxide enhancing salt of salicylamide O-acetic acid, an organic nitric oxide enhancing salt of sulindac, an organic nitric oxide enhancing salt of suprofen, an organic nitric oxide enhancing salt of suxibuzone, an organic nitric oxide enhancing salt of tiaprofenic acid, an organic nitric oxide enhancing salt of tolmetin, an organic nitric oxide enhancing salt of xenbucin, an organic nitric oxide enhancing salt of ximoprofen, an organic nitric oxide enhancing salt of zaltoprofen or an organic nitric oxide enhancing salt of zomepirac; and the organic nitric oxide enhancing salt of NSAID of Formula II is an organic nitric oxide enhancing salt of aspirin, an organic nitric oxide enhancing salt of acemetcin, an organic nitric oxide enhancing salt of bumadizon, an organic nitric oxide enhancing salt of carprofenac, an organic nitric oxide enhancing salt of clidanac, an organic nitric oxide enhancing salt of diflunisal, an organic nitric oxide enhancing salt of enfenamic acid, an organic nitric oxide enhancing salt of fendosal, an organic nitric oxide enhancing salt of flufenamic acid, an organic nitric oxide enhancing salt of flunixin, an organic nitric oxide enhancing salt of gentisic acid, an organic nitric oxide enhancing salt of ketorolac, an organic nitric oxide enhancing salt of meclofenamic acid, an organic nitric oxide enhancing salt of mefenamic acid, an organic nitric oxide enhancing salt of mesalamine, an organic nitric oxide enhancing salt of niflumic acid, an organic nitric oxide enhancing salt of salsalate, an organic nitric oxide enhancing salt of tolfenamic acid or an organic nitric oxide enhancing salt of tropensin.

4. The compound of claim 1, wherein the compound of Formula (I) is an organic nitric oxide enhancing salt of acemetacin, an organic nitric oxide enhancing salt of aceclofenac of Formula (III), an organic nitric oxide enhancing salt of alclofenac of Formula (IV), an organic nitric oxide enhancing salt of bromfenac of Formula (V), an organic nitric oxide enhancing salt of carprofen of Formula (VI), an organic nitric oxide enhancing salt of diclofenac of Formula (VII), an organic nitric oxide enhancing salt of etodolac of Formula (VIII), an organic nitric oxide enhancing salt of fenbufen of Formula (IX), an organic nitric oxide enhancing salt of fenoprofen of Formula (X), an organic nitric oxide enhancing salt of flurbiprofen of Formula (XI), an organic nitric oxide enhancing salt of ibuprofen of Formula (XII), an organic nitric oxide enhancing salt of indomethacin of Formula (XIII), an organic nitric oxide enhancing salt of ketoprofen of Formula (XIV), an organic nitric oxide enhancing salt of loxoprofen of Formula (XV), an organic nitric oxide enhancing salt of naproxen of Formula (XVI), an organic nitric oxide enhancing salt of oxaprozin of Formula (XVII), an organic nitric oxide enhancing salt of pirozolac of Formula (XVIII), an organic nitric oxide enhancing salt of sulindac of Formula (XIX), an organic nitric oxide enhancing salt of suprofen of Formula (XX), an organic nitric oxide enhancing salt of tolmetin of Formula (XXI); and the organic nitric oxide enhancing salt of NSAID of Formula II is an organic nitric oxide enhancing salt of aspirin of Formula (XXII), an organic nitric oxide enhancing salt of acemetcin of Formula (XXIII), an organic nitric oxide enhancing salt of diflunisal of Formula (XXIV), an organic nitric oxide enhancing salt of enfenamic acid of Formula (XXV), an organic nitric oxide enhancing salt of flufenamic acid of Formula (XXVI), an organic nitric oxide enhancing salt of gentisic acid of Formula (XXVII), an organic nitric oxide enhancing salt of ketorolac of Formula (XXVIII), an organic nitric oxide enhancing salt of meclofenamic acid of Formula (XXIX), an organic nitric oxide enhancing salt of mefenamic acid of Formula (XXX), an organic nitric oxide enhancing salt of mesalamine of Formula (XXXI), an organic nitric oxide enhancing salt of salsalate of Formula (XXXII) or an organic nitric oxide enhancing salt of tolfenamic acid of Formula (XXXIII);
and the compound of Formula (III) is: 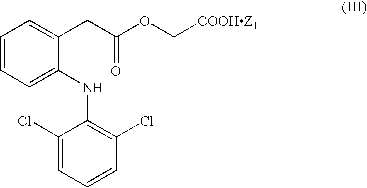
and the compound of Formula (IV) is: 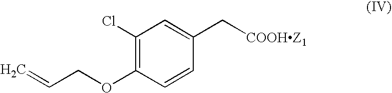
and the compound of Formula (V) is: 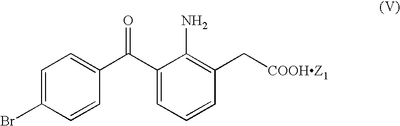
and the compound of Formula (VI) is: 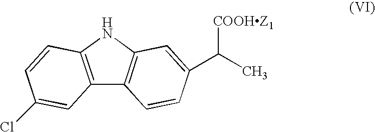
and the compound of Formula (VII) is: 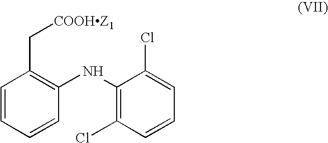
and the compound of Formula (VIII) is: 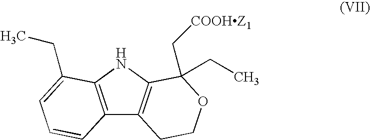
and the compound of Formula (IX) is: 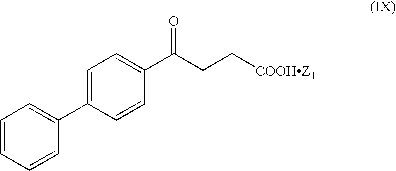
and the compound of Formula (X) is: 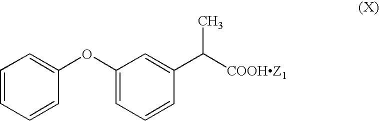
and the compound of Formula (XI) is: 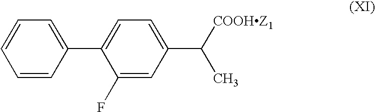
and the compound of Formula (XII) is: 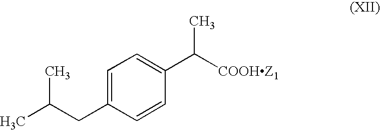
and the compound of Formula (XIII) is: 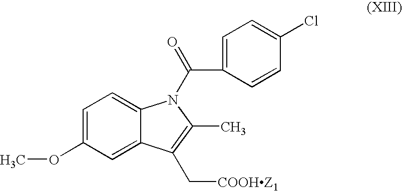
and the compound of Formula (XIV) is: 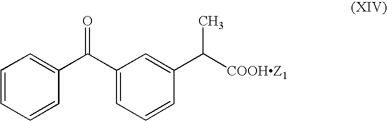
and the compound of Formula (XV) is: 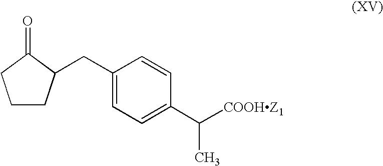
and the compound of Formula (XVI) is: 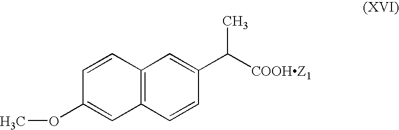
and the compound of Formula (XVII) is: 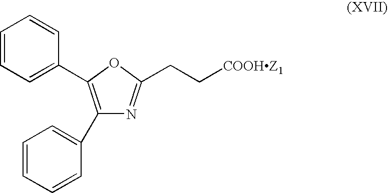
and the compound of Formula (XVIII) is: 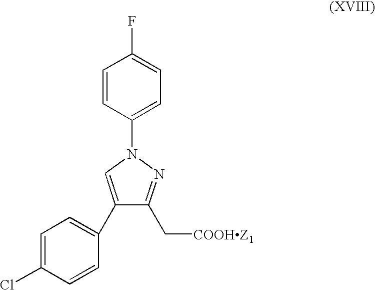
and the compound of Formula (XIX) is: 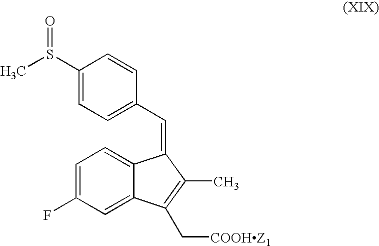
and the compound of Formula (XX) is: 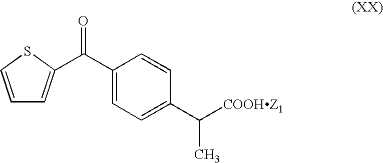
and the compound of Formula (XXI) is: 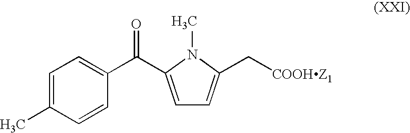
and the compound of Formula (XXII) is: 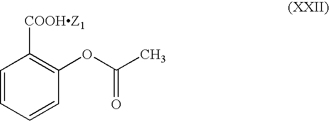
and the compound of Formula (XXIII) is: 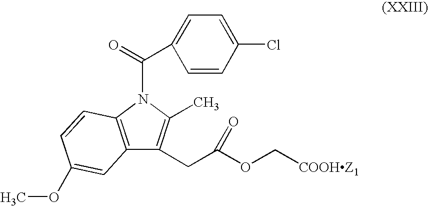
and the compound of Formula (XXIV) is: 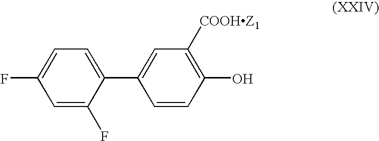
and the compound of Formula (XXV) is: 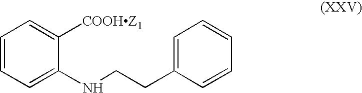
and the compound of Formula (XXVI) is: 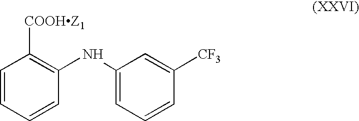
and the compound of Formula (XXVII) is: 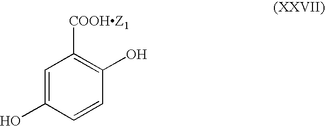
and the compound of Formula (XXVIII) is: 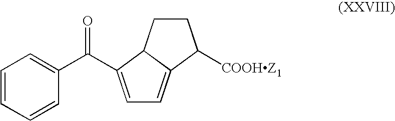
and the compound of Formula (XXIX) is: 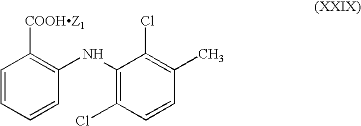
and the compound of Formula (XXX) is: 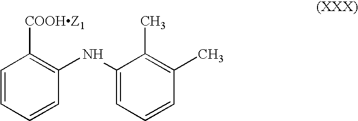
and the compound of Formula (XXXI) is: 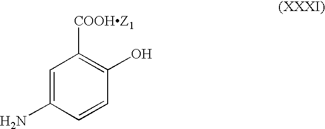
and the compound of Formula (XXXII) is: 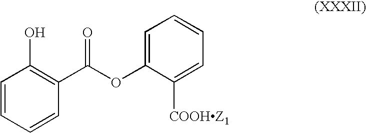
and the compound of Formula (XXXIII) is: 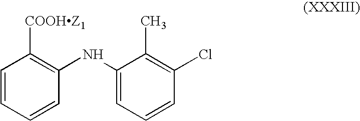
Z₁ is: 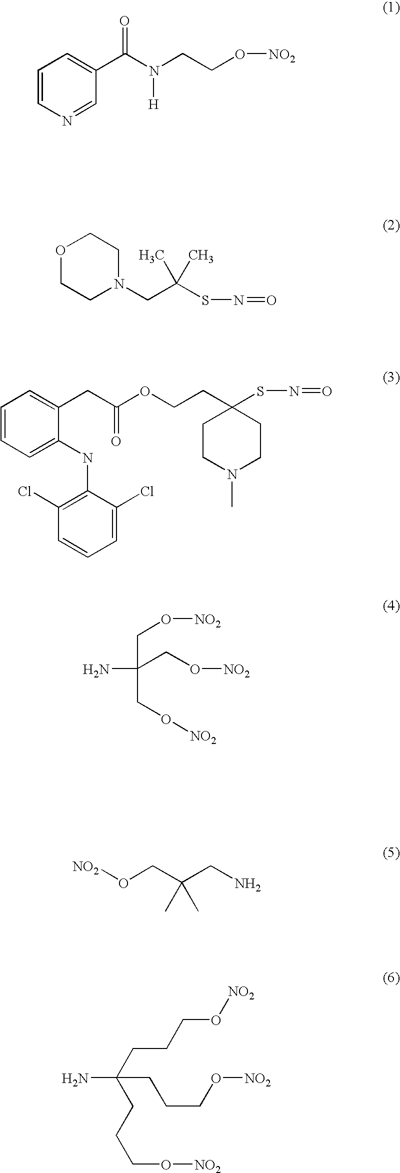 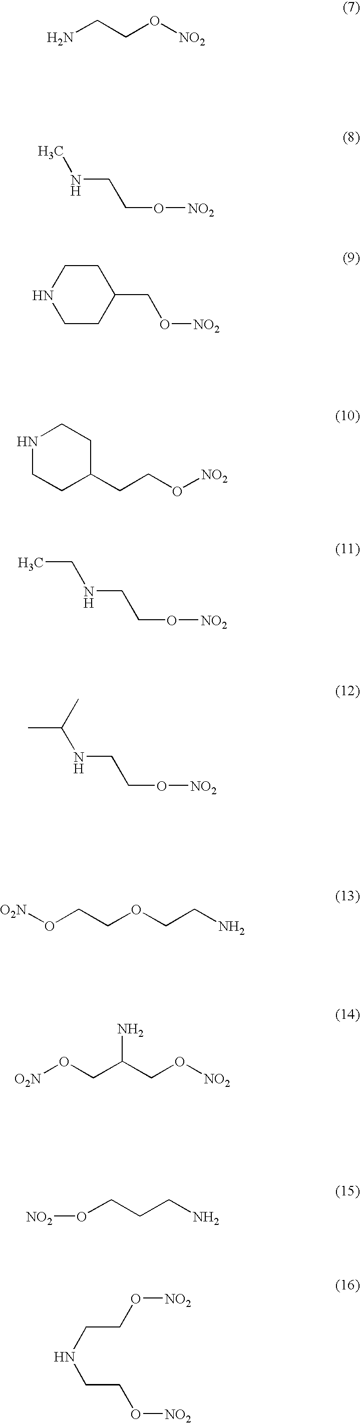 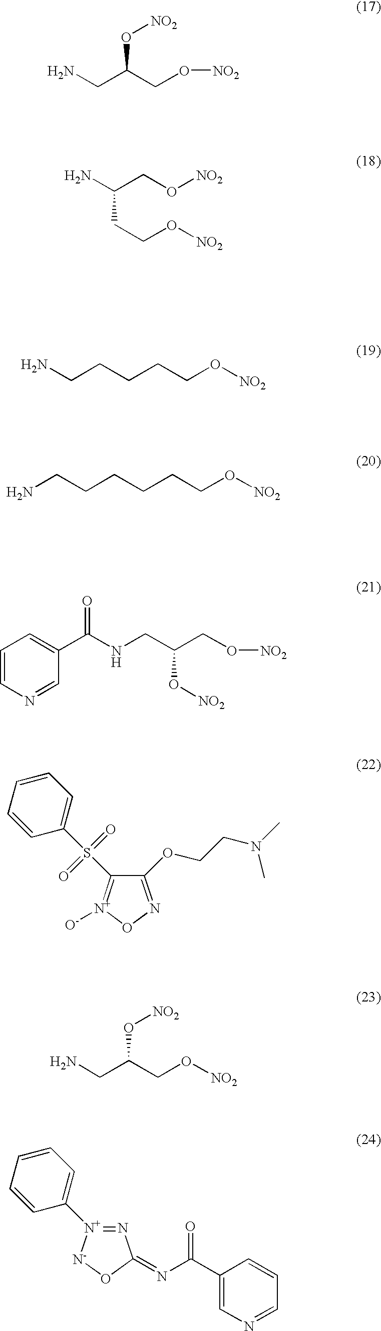 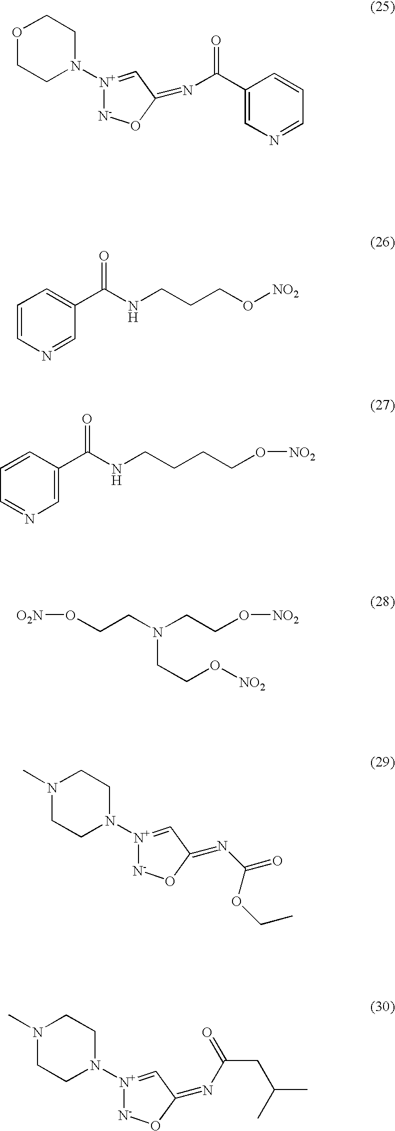 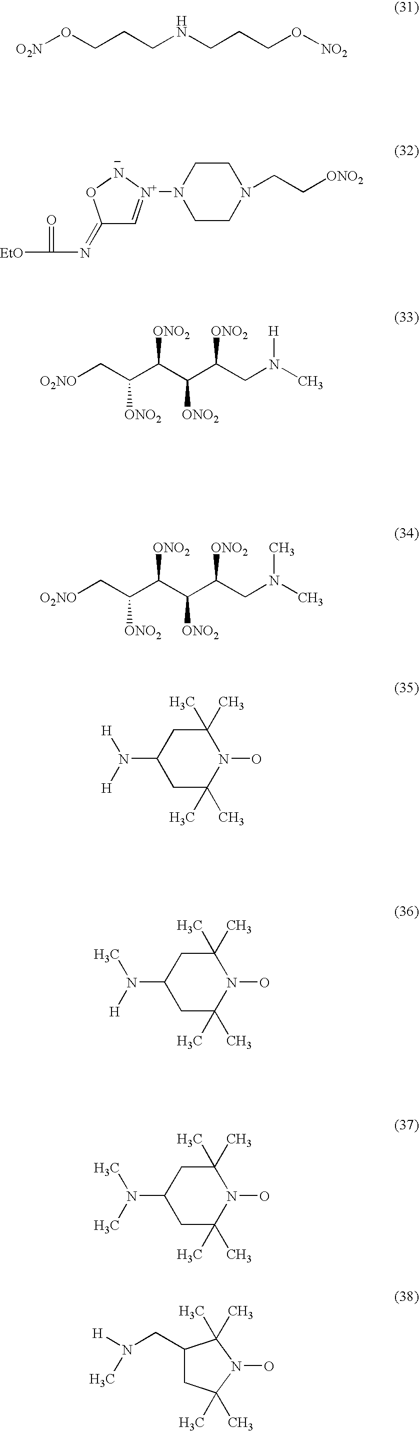 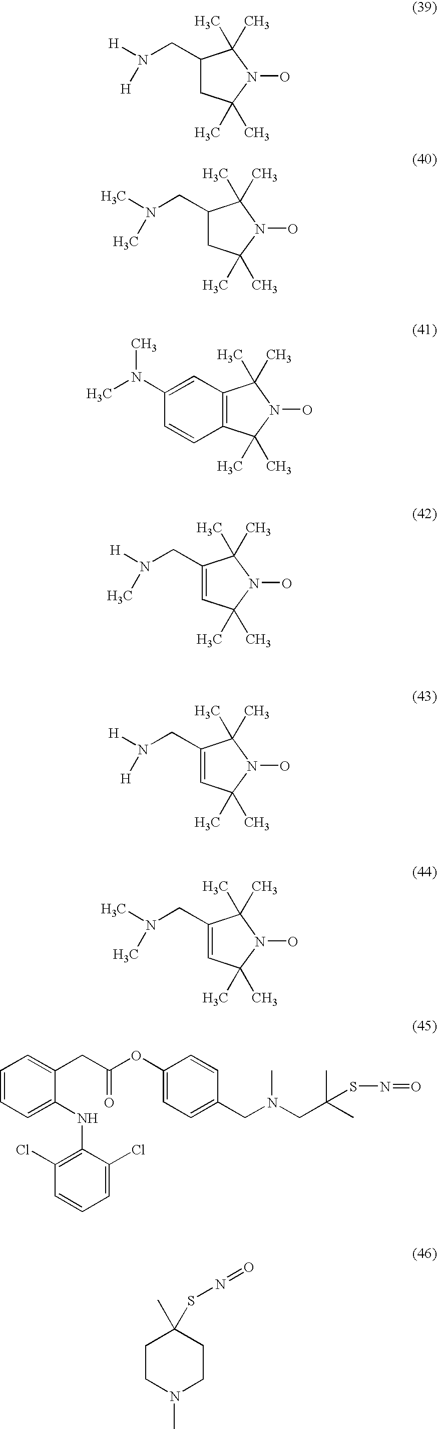 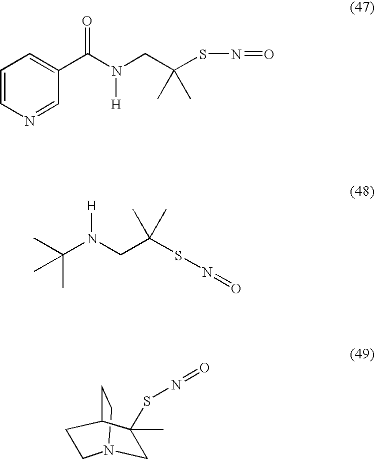
with the proviso that the compounds of Formula (III) and (XXXIII) must contain at least one organic nitric oxide enhancing compound linked via a salt bridge (i.e., . or ▪) to at least one carboxylic acid group in the compounds of Formula (III) and (XXXIII).

5. A method for treating or reducing inflammation, pain or fever in a patient in need thereof comprising administering to the patient a therapeutically effective amount of the composition of claim 2.

6. A method for treating a gastrointestinal disorder in a patient in need thereof comprising administering to the patient a therapeutically effective amount of the composition of claim 2.

7. The method of claim 6, wherein the gastrointestinal disorder is an inflammatory bowel disease, Crohn's disease, gastritis, irritable bowel syndrome, constipation, ulcerative colitis, a peptic ulcer, a stress ulcer, a bleeding ulcer, gastric hyperacidity, dyspepsia, gastroparesis, Zollinger-Ellison syndrome, gastroesophageal reflux disease, a bacterial infection, short-bowel (anastomosis) syndrome, or a hypersecretory state associated with systemic mastocytosis or basophilic leukemia and hyperhistaminemia.

8. A method for facilitating wound healing in a patient in need thereof comprising administering to the patient a therapeutically effective amount of the composition of claim 2.

9. The method of claim 8, wherein the wound is an ulcer.

10. A method for treating or reversing gastrointestinal, renal and/or respiratory toxicity in a patient in need thereof comprising administering to the patient a therapeutically effective amount of the composition of claim 2.

11. A method for treating an inflammatory disease in patient in need thereof comprising administering to the patient a therapeutically effective amount of the composition of claim 2, wherein the inflammatory disease is a cardiovascular disorder, reperfusion injury to an ischemic organ, angiogenisis, arthritis, asthma, bronchitis, premature labor, tendinitis, bursitis, an autoimmune disease, an immunological disorder, a skin-related condition, neoplasia, an inflammatory process in a disease, pulmonary inflammation, a central nervous system disorder, allergic rhinitis, respiratory distress syndrome, endotoxin shock syndrome, a microbial infection, a bacterial-induced inflammation, a viral induced inflammation, a urinary disorder, a urological disorder, endothelial dysfunction, organ deterioration, tissue deterioration, a sexual dysfunction or activation, adhesion and infiltration of neutrophils at the site of inflammation.

12. A method for treating an ophthalmic disorder; treating a peripheral vascular disease; treating a diseases resulting from oxidative stress; treating an endothelial dysfunction; or treating a disease caused by endothelial dysfunctions in a patient in need thereof comprising administering to the patient an effective amount of the composition of claim 2.

13. The composition of claim 2, further comprising (i) at least one therapeutic agent; (ii) at least one nitric oxide enhancing compound; or (iii) at least one therapeutic agent and at least one nitric oxide enhancing compound.

14. The composition of claim 13, wherein the therapeutic agent is a steroid, a cyclooxygenase-2 inhibitor, a nonsteroidal antiinflammatory compound, a 5-lipoxygenase inhibitor, a leukotriene B₄ receptor antagonist, a leukotriene A₄ hydrolase inhibitor, a 5-HT agonist, an anti-hyperlipidemic compound, a H₂ antagonist, an antineoplastic agent, an antiplatelet agent, a thrombin inhibitor, a thromboxane inhibitor, a carbonic anhydrase inhibitor, a decongestant, a diuretic, a sedating or non-sedating anti-histamine, an inducible nitric oxide synthase inhibitor, an opioid, an analgesic, an Helicobacter pylori inhibitor, a phosphodiesterase inhibitor, a proton pump inhibitor, an isoprostane inhibitor, a compound used for the treatment of glaucoma, and combinations of two or more thereof.

15. The composition of claim 13, wherein the nitric oxide enhancing compound is selected from the group consisting of a S-nitrosothiol, a nitrite, a nitrate, a S-nitrothiol, a sydnonimine, a NONOate, a N-nitrosoamine, a N-hydroxyl nitrosamine, a nitrosimine, a diazetine dioxide, an oxatriazole 5-imine, an oxime, a hydroxylamine, a N-hydroxyguanidine, a hydroxyurea, a furoxan or a nitroxide.

16. The method of claims 6, 7, 8, 10, 11 or 12, further comprising administering (i) at least one therapeutic agent; (ii) at least one nitric oxide enhancing compound or (iii) at least one therapeutic agent and at least one nitric oxide enhancing compound.

17. A kit comprising at least one compound of claim 1.

18. The kit of claim 17, further comprising further comprising (i) at least one therapeutic agent; (ii) at least one nitric oxide enhancing compound; or (iii) at least one therapeutic agent and at least one nitric oxide enhancing compound.

19. The kit of claim 18, wherein the (i) at least one therapeutic agent; (ii) at least one nitric oxide enhancing compound; or (iii) at least one therapeutic agent and at least one nitric oxide enhancing compound are in the form of separate components in the kit.
